SEQUENCE LISTING (2 sequences)

SEQ ID NO: 1 - protein, 260 aa, Artificial Sequence
  Synthetic
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 2 - DNA, 783 bp, Artificial Sequence
  Synthetic
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataa

(19) United States
(12) Patent Application Publication
(10) Pub. No.: US 2023/0081321 A1
(43) Pub. Date: Mar. 16, 2023

(54) CARBONIC ANHYDRASE II COMPOSITIONS AND METHODS OF USE THEREOF

(71) Applicant: University of Florida Research Foundation, Incorporated, Gainesville, FL (US)

(72) Inventors: Jacob T. Andring, Gainesville, FL (US); Robert McKenna, Gainesville, FL (US)

(73) Assignee: University of Florida Research Foundation, Incorporated, Gainesville, FL (US)

(21) Appl. No.: 17/792,310

(22) PCT Filed: Jan. 14, 2021
(86) PCT No.: PCT/US21/13416

§ 371 Date: Jul. 12, 2022

Related U.S. Application Data
(60) Provisional application No. 62/961,147, filed on Jan. 14, 2020.
(60) Provisional application No. 63/008,607, filed on Apr. 10, 2020.

(51) Int. Cl. A61K 38/51; A61K 33/34; A61K 45/06

(52) U.S. Cl. CPC A61K 38/51; C12Y 402/01001; A61K 33/34; A61K 45/06

(57) ABSTRACT

Provided herein are compositions of carbonic anhydrase and inhibitors thereof for the treatment of subjects with certain conditions such as heart disease.

### RELATED APPLICATIONS

This application claims the benefit under 35 U.S.C. § 119(e) of the filing date of U.S. Provisional Application No. 62/961,147, entitled “CU CARBONIC ANHYDRASE II AS A THERAPEUTIC AGENT FOR HEART DISEASE”, filed Jan. 14, 2020, and U.S. Provisional Application No. 63/008,607, entitled “CU CARBONIC ANHYDRASE II AS A THERAPEUTIC AGENT FOR HEART DISEASE”, filed Apr. 10, 2020, the contents of each of which are incorporated herein by reference in their entireties.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jan. 8, 2021, is named U119670082WO00-SEQ.txt and is 3.83 kilobytes in size.

### BACKGROUND OF THE INVENTION

Carbonic anhydrase II (CAII) is one of at least fifteen forms of human a carbonic anhydrases, and is present in the blood of subjects (e.g., human subjects). However, it's involvement in disease is not yet fully understood.

### SUMMARY OF THE INVENTION

This disclosure is based, at least in part, on the discovery of the various roles of the different forms of carbonic anhydrase II (CAII) in various diseases. These various roles involve CAII's nitrite reductase activity, as well as its nitrite reductase activity. This disclosure describes various compositions of CAII as well as CAII inhibitors to treat disease, and methods of making such compositions and using such compositions (e.g., for treating disease).

This disclosure is based, at least in part, on the discovery that carbonic anhydrase, which is found in blood, has nitrite reductase activity when bound to copper in a particular configuration. Nitrite reductase activity is useful for treating conditions such as hypertension, heart conditions, muscular atrophy, or any condition that can be relieved by causing vasodilation.

Accordingly, provided herein is a composition comprising carbonic anhydrase II (CAII) and copper, wherein the composition has nitrite reductase activity. In some embodiment, the copper is bound to the carbonic anhydrase. In some embodiments, His94, His96, and His119 of the CAII corresponding to amino acids in SEQ ID NO: 1 are bound to a copper atom, and His4, His3, and Ser2 of the CAII corresponding to amino acids in SEQ ID NO: 1 are bound to a copper atom. In some embodiments, any one of the CAII comprising compositions disclosed herein further comprises a pharmaceutically acceptable carrier.

In some embodiments, a composition of CAII comprises a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a copper atom through His94, His96, and His119 of the CAII.

In some embodiments, a composition of CAII comprises a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a copper atom through His4, His3, and Ser2 of the CAII.

In some embodiments, a composition of CAII comprises a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a first copper atom through His94, His96, and His119 of the CAII, and a second copper atom through His4, His3, and Ser2 of the CAII.

In some aspects, provided herein is a method of making a composition comprising CAII having nitrite reductase activity. In some embodiments, a method of making the composition of claim 1, comprises: purifying CAII from a blood sample or culture of bacteria; chelating metal ions from the purified CAII; and incubating the purified CAII from which metal ions are chelated with copper at a molar ratio of 0.1:1 to 1:1 of CAII to copper. In some embodiments, a chelating metal ions from the purified CAII comprises incubating the purified CAII with pyridine-2,6-dicarboxylic acid (DPA).

Also provided herein is a composition comprising CAII, wherein the composition is prepared by: purifying CAII from a blood sample or culture of bacteria; chelating metal ions from the purified CAII; and incubating with copper at a molar ratio of 0.1:1 to 1:1 of CAII to copper.

Provided herein are methods of treating a subject suffering from or is at risk of suffering from a condition that can be affected by vasodilation. In some embodiments, a method comprises administering to a subject the composition of any one of the compositions of CAII having nitrite reductase activity. In some embodiments, a subject that is administered any one of the CAII compositions disclosed herein suffers from or is at risk of suffering from a condition that can be relieved by causing vasodilation. In some embodiments, a condition that can be relieved by causing vasodilation is hypertension, pulmonary hypertension, a heart condition, erectile dysfunction, or muscular atrophy. In some embodiments, a heart condition is heart failure, angina, coronary artery disease, or myocardial infarction. In some embodiments, hypertension is primary hypertension or secondary hypertension, wherein the secondary hypertension is secondary to eclampsia, preeclampsia, renovascular disease or renal disease, sleep apnea, or endocrine abnormalities. In some embodiments, the composition is administered at a dose sufficient to increase the amount of copper-bound CAII in the subject by 10% or more.

In some aspects, provided herein is a method comprising administering to a subject one or more inhibitors of carbonic anhydrase II (CAII). In some embodiments, the one or more inhibitors of CAII increase the nitrite reductase activity of Cu bound CAII (e.g., by at least 5%, by at least 10%, by at least 15%, by at least 20%, by at least 30%, by at least 40%, by at least 50%, by at least 60%, by at least 70%, by at least 80%, by at least 90%, by at least 100%, by at least 200%, by at least 300% or more). In some embodiments, the one or more inhibitors of CAII improve the nitrite reductase activity of Cu bound CAII compared to CAII that is not treated by the inhibitor. In some embodiments, the CAII that interacts with the inhibitor is bound to Cu. In some embodiments, the CAII that interacts with the inhibitor is bound to Zn. In some embodiments, one or more inhibitors preferentially inhibits CAII bound to Zn relative to CAII bound to Cu. In some embodiments, one or more inhibitors of carbonic anhydrase II that are administered to a subject is/are sulfonamide-based carbonic anhydrase inhibitors. In some embodiments, one or more inhibitors of carbonic anhydrase II that are administered to a subject is/are sulfonamide-based carbonic anhydrase inhibitors (e.g., acetazolamide, methazolamide, ethoxzolamide, dichlorphenamide, dorzolamide, brinzolamide, topiramate, celecoxib, sulpiride, sulthiame, valdecoxib, zonisamide, irosustat, an esterone sulfamate, or a benzyl-sulfonamide compound).

In some embodiments, a subject that is administered one of more inhibitors of CAII suffers from or is at risk of suffering from a condition that can be relieved by causing vasodilation. In some embodiments, a condition that can be relieved by causing vasodilation is hypertension, pulmonary hypertension, a heart condition, erectile dysfunction, or muscular atrophy. In some embodiments, a heart condition is heart failure, angina, coronary artery disease, or myocardial infarction. In some embodiments, hypertension is primary hypertension or secondary hypertension, wherein the secondary hypertension is secondary to eclampsia, preeclampsia, renovascular disease or renal disease, sleep apnea, or endocrine abnormalities.

The inventors of the present disclosure have found that CAII has esterase activity by which CAII can degrade NSAIDs (e.g., aspirin) such as those administered to subjects with heart conditions (e.g., subjects having suffered, are suffering, or are at risk of suffering a myocardial infarction, stroke, or Raynaud's phenomenon). For example, CAII converts aspirin to the acetylated form of aspirin. This results in a lower concentration of aspirin in the body that can perform its intended function (e.g., inhibition of COX). Therefore, by inhibiting the esterase activity of CAII, subjects who have been administered aspirin can have a higher amount of aspirin to perform the intended function (and thus a higher half-life of aspirin).

Accordingly, provided herein is a method comprising administering to a subject who is administered or is going to be administered a nonsteroidal anti-inflammatory drug (NSAID) an inhibitor of carbonic anhydrase II (CAII), wherein the CAII has esterase activity. In some embodiments, an NSAID is aspirin or ibuprofen. In some embodiments, a NSAID is aspirin.

In some embodiments, an inhibitor of carbonic anhydrase II is a sulfonamide-based carbonic anhydrase inhibitor. In some embodiments, an inhibitor of carbonic anhydrase II is acetazolamide, methazolamide, ethoxzolamide, dichlorphenamide, dorzolamide, brinzolamide, topiramate, celecoxib, sulpiride, sulthiame, valdecoxib, zonisamide, irosustat, esterone sulfamate, or a benzyl-sulfonamide compound.

In some embodiments, a subject to whom an inhibitor of CAII is administered to target CAII esterase activity is a subject who has experienced a myocardial infarction, stroke, or Raynaud's phenomenon. In some embodiments, a subject is administered the CAII inhibitor simultaneously with being administered the NSAID, or within 4 hours of being administered the NSAID.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1B show the structure of Zn- and Cu-CAII: Active site and water network. FIG. 1A shows Zn-CAII. The zinc metal stabilized by three histidines (H94, H96, and H119). H64 depicted in its dual “in” and “out” conformations. The N terminus (residues 1-4) disordered. The substrate CO₂ shown bound adjacent to the zinc, stabilized by the hydrophobic pocket. FIG. 1B shows Cu-CAII. The copper metal (T-2 site) stabilized by the same three histidines as the zinc. Also, H64 was observed in dual conformations. The NO₂⁻ bound with an oxygen and nitrogen interacting with the copper. The water network resembles that observed in Zn-CAII, with the exception of the extended water network (W4 and W5), creating a hydrogen-bonding network spanning from the N terminus to the active site. The N terminus is ordered, forming a pseudo porphyrin ring around a second copper (T-2 site). The catalytic metals are depicted as spheres zinc (large sphere in FIG. 1A) and copper (large sphere in FIG. 1B). The hydrophobic residues (I91, V121, F131, V135, L141, V143, L198, P202, L204, V207, and W209) are shown vertically striped and the hydrophilic residues (N62, H64, N67, Q92, T199, and T200) shown horizontally striped. The active site solvent network: W1, W2, W3a, and W3b, are labeled as such and are depicted as small spheres, and the extended water network W4 and W5, are shown in the Cu-CAII substituted structure.

FIGS. 2A-2B show the active site of Zn- and Cu-CAII (T-2 site) with bound substrate, CO2 and NO2-, respectively. FIG. 2A shows CO2 binding site in Zn-CAII active site (adapted from PDB: 3KS3, 5YUI). CO2 binds adjacent to the zinc, approximately 2.8 Å from the catalytic Zn-bound solvent. The CO2 is stabilized via interactions with residues V121, V143, L198, and W209. T199 also forms a hydrogen bond with CO2 via its nitrogen. FIG. 2B NO2- binding site in Cu-CAII active site. NO2- binds directly to the copper, displacing the Cu-bound solvent. It binds in a “side-on” conformation via an oxygen and nitrogen 2.1 and 2.8 Å from the copper, respectively. However, solvent W1 retains its position and forms hydrogen bonds with an oxygen of NO2-. T199 forms two hydrogen bonds with the bound NO2- while L198 also forms stabilizing interactions. The catalytic metals are depicted as spheres, zinc (large sphere in FIG. 2A) and copper (large sphere in FIG. 2B). The active site solvent molecules are depicted as small spheres.

FIGS. 3A-3C show the N terminus of Cu-CAII (T-1 site). FIG. 3A shows the T-1 copper is stabilized by the N terminus of Cu-CAII by residues S2, H3, and H4. The copper is also hydrogen-bounded to solvent molecule facing H64 (presumably for electron transfer to the T-2 site). Interestingly, residue H3 adopts dual conformations, one away and one towards the copper. FIG. 3B shows the structure of an iron containing porphyrin ring from Pseudomonas aeruginosa nitrite reductase (PDB 1N15). FIG. 3C shows the superposition of Cu-CAII N terminus with the Pseudomonas aeruginosa nitrite reductase heme, R.M.S.D. of 0.27 Å. It is important to note that the N terminus T-1 site is less ordered in comparison to the rest of the structure. The occupancy and B factor of the T-1 site is 0.71 and 29.1 Å2, respectively, while for the T-2 site the occupancy and B factor were 1.00 and 11.4 Å2, respectively. This is because the N terminus needs to be transient, only forming when need in the blood, effectively acting as an on/off switch. Also, this transient feature allows rapid metal exchange, allowing trace metals in the blood to quickly bind and disassociate for electron transfer.

FIGS. 4A-4E shows the proposed Cu-CAII nitrite reductase mechanism. FIG. 4A shows Cu-CAII in resting state, with a copper-bound solvent molecule. T199 is slightly acidic due to interactions with D106 allowing T199 to stabilize the solvent molecule. W1 is stabilized via hydrogen-bonding to T200 and W2. FIG. 4B shows NO2- entering the active site, displacing the copper bound solvent. NO2- binds in a “hat” conformation, with both oxygen atoms coordinating to the copper. One oxygen is primed for catalysis via hydrogen-bonding to the hydroxyl of T199 and W1. FIG. 4C shows intermolecular electron transfer from the T-1 copper site, donating an electron to the T-2 copper site, generating a Cu+ cation in the T-2 active site. This triggers a binding mode change in NO2- from “hat” to “side” on” coordination. One oxygen is uncoordinated from the copper and stabilized via the nitrogen from T199 while the other oxygen retains hydrogen-bonding to T199 and W1. FIG. 4D shows the reduction of nitrite begins via an electron donation from Cu+, resulting in a cascade of electron rearrangement and the regeneration of Cu2+. The primed oxygen accepts two protons from W1 and the acidic hydroxyl of T199 forming a bound water molecule. FIG. 4E shows the nitrite molecule as reduced to nitric oxide and transiently bound to the Cu2+ cation along with the generated water molecule. As the water molecule forms, the nitric oxide is released from the copper. More protons are shuttled into the active site via the CA proton shuttle H64 and the necessary catalytic protons are replenished regenerating the resting state in FIG. 4A.

FIG. 5 shows an illustration of human carbonic anhydrases. CAII is circled.

FIG. 6 shows the general carbonic anhydrase mechanism of CAII.

FIGS. 7A-7D show various illustrations relating to nitrite and nitric oxide. FIG. 7A shows the structure of nitric oxide which stimulates smooth muscle relaxation through the activation of guanylate cyclase and is responsible for vasodilation. FIG. 7B shows the mechanism of nitric oxide synthetase. FIG. 7C shows a schematic of pulmonary arterial hypertension (PAH), which is one of many heart conditions that can benefit from the effects of vasodilation. FIG. 7D shows the nitrate reductase pathway. In humans, this pathway is activated under hypoxia. It represents an alternative pathway for NO generation but the enzyme/mechanism is unknown. However, bacteria have multiple copper containing enzymes responsible for nitrite reduction.

FIGS. 8A-8C show the results of experiments to determine if carbonic anhydrase has reductase activity using a NO sensitive electrode from Aamand, et al. (Am. J. Physiol. Heart Circ. Physiol. 297: H2068-H2074, 2009). FIG. 8A shows a NO sensitive electrode. FIG. 8B shows a measurement of NO when at pH 7.2, 100 uM KNO2 was added to 100 uM CAII, and then to this reaction mixture, 250 uM Dorzolamide was added at 8 minutes. FIG. 8C shows a measurement of NO when at pH of 5.9, 100 uM KNO2 was added to 100 uM CAII, and then to this reaction mixture, 250 uM Dorzolamide was added at 8 minutes.

FIGS. 9A-9C show that the Zn CAII does not have the nitrite reductase activity in experiments with carbonic anhydrase in the presence and absence of EDTA, which can chelate copper but not zinc. FIG. 9A shows molecular modeling of the interaction between Zn CAII and nitrite in the presence of a CAII inhibitor. FIG. 9B shows NO concentration, measured via NO-sensitive electrode, over time in a reaction vessel at pH 5.9 with 100 uM CAII to which 100 uM KNO₂ was added. The reaction mixture was spiked with 250 uM dorzolamide at the time indicated by the arrow. FIG. 9C shows NO concentration over time, measured via membrane inlet mass spectrometry (showing 30 m/z signal, indicative of NO formation), in a reaction vessel at pH 5.9 with 100 uM CAII.

FIG. 10 shows a chemical structure of ethylenediaminetetraacetic acid (EDTA).

FIGS. 11A-11B show copper binding sites for His4 and His64. FIG. 11A shows the active site of CAII bound to both zinc and copper (Zn,Cu-CAII), in which the zinc ion is coordinated by His94, His96 and His119 and an oxygen molecule, and the copper ion is coordinated by His64 and His4 (Ferraroni, et al., J. Enzyme Inhib. Med. Chem., 33(1): 999-1005, 2018). In this instance, coordination with another metal aside from copper (e.g., zinc, mercury, etc.) was deemed to be necessary for CAII functionality. FIG. 11B shows CAII in which one metal coordination site (His94/96/119 site) is occupied by zinc and the other (His4/His3/Ser2 site) is occupied by copper, as provided here.

FIGS. 12A-12B show that CAII shows similarity to bacterial nitrite reductases, as both sites need to be occupied by copper to activate nitrite reductase activity. FIG. 12A shows copper binding sites within nitrite reductase (Li et al., Biochemistry 2015 54(5):1233-1242). FIG. 12B shows overlays of substrate-binding sites of Zn carbonic anhydrase and Cu nitrite reductase (Strange, et al., Nat. Struct. Biol. 1995, 2(4):287-292).

FIG. 13 shows the generation of Cu-substituted CAII in preparation for X-ray crystallography.

FIGS. 14A-14C show the results of X-ray crystallography for Apo CAII (FIG. 14A), Zn CAII (FIG. 14B), and Cu CAII (FIG. 14C). In Apo CAII, the active site is empty and the N-terminus is disordered. In Zn CAII, the active site with zinc chelated by H94, H96, and H119 and the N-terminus is disordered with density only for H4. In, Cu CAII with metal bound at both the T1 and T2 sites, the N-terminus is ordered around the copper atom, forming a ATCUN binding site.

FIG. 15 shows the electron density (0.8σ) of the ordered N-terminus of Cu-substituted CAII, demonstrating a novel copper binding site not utilizing His64.

FIG. 16 shows the amino terminal copper and nickel binding motif from Nettles et al. (Inorg Chem., 2015; 54(12):5671). Nettles et al. predicted that the N terminus of CAII could gain order around a metal ion. However, Nettles et al. could not accurately predict the order or coordination mode.

FIG. 17 shows results from X-ray crystallography studies, demonstrating endogenous NO₂⁻ bound to Cu-CAII T2 site, as is seen in bacterial nitrate reductase T2 sites.

FIGS. 18A-18C show NO₂⁻ bound Zn-CAII. FIGS. 18A and 18B show NO₂⁻ bound Zn-CAII after soaking with NO₂⁻. FIG. 18C shows superposition of CO₂ binding and NO2 binding, demonstrating that NO₂⁻ binds the same pocket as CO₂ in Zn-bound CAII.

FIGS. 19A-19C show NO bound Cu-Carbonic Anhydrase II after soaking with NO₂⁻. FIGS. 19A and 19B show NO bound Cu-Carbonic Anhydrase II after soaking with NO2 FIG. 19C shows superposition of NO₂⁻ soaked Zn-CAII and NO₂⁻ soaked Cu-CAII, and demonstrates different ligand as well as different binding mode between the two metal ion CAs.

FIG. 20 shows a proposed mechanism of nitrite reduction catalyzed by copper-containing nitrite reductases, as postulated by Li et al. (Biochemistry 2015, 54(5): 1233-1242).

FIG. 21 shows the structure of CAII complexed with salicylic acid. Hydrophobic face of CAII is shown vertically striped while the hydrophilic face is shown horizontally striped. Zinc depicted as a magenta sphere with critical binding residues shown in sticks. Bound salicylic acid is shown in green sticks. Top insert, active site with SA interactions and hydrogen bonds shown in dashes. Bottom insert, electron density for SA shown as blue mesh. PDB: 6UX1

FIG. 22 shows the inhibition curve of CAII with SA. Calculated IC50 of 6.6 mM. The error bars represent the standard deviation of 3 kinetic experiments performed.

FIG. 23 shows the structure of Aspirin modeled into the active site of CAII. Hydrophobic face of CAII is shown as a vertically striped surface while the hydrophilic face is shown horizontally striped. Zinc depicted as a large sphere with critical residues shown in sticks. Bound Aspirin is shown as a stick model structure. V134 and W204 unlabeled for clarity.

FIGS. 24A-24E show the proposed mechanism of CA esterase function, that converts Aspirin to SA.

FIG. 25 shows the reaction of CAII and aspirin, resulting in SA.

FIG. 26 shows T1 and T2 Copper Binding Sites in Achromobacter cycloclastes Cu Nitrite Reductase. T1 and T2 copper sites are shown with endogenously bound NO₂⁻ in T2 site. Adapted from Li et al., Biochemistry 2015 54(5):1233-1242 with permissions.

FIG. 27 shows X-ray absorption edge spectra of Zn-CAII. Zn-CAII shows the expected absorption edge at ˜9659 eV, indicative of zinc bound.

FIG. 28 shows X-ray absorption edge spectra of Apo-CAII. Apo-CAII shows no absorption edge around 8979 eV or 9659 eV, indicating no metal present.

FIG. 29 shows X-ray absorption edge spectra of Cu-CAII. Cu-CAII shows the copper absorption edge at ˜8979 eV but not the zinc edge at 9659 eV, indicating only copper bound.

FIG. 30 shows dissolved oxygen over time in perfusate passed through a mouse heart in a Langendorff preparation (retrograde perfusion via the aorta). “O₂ in” refers to the oxygen in the perfusate before the solution passes through the heart or the oxygen in the acetazolamide solution. “O₂ out” refers to the oxygen in the perfusate after it passes through the heart. “Water” indicates data recorded from air-saturated water, to check oxygraphy. “No oxygenation” indicates data recorded for technical checks. Acetazolamide (1 mM) was added to the solution twice over the course of the measurements.

FIG. 31 shows dissolved oxygen over time in perfusate passed through a mouse heart in a Langendorff preparation (retrograde perfusion via the aorta). “O₂ in” refers to the oxygen in the perfusate before the solution passes through the heart or the oxygen in the dorzolamide solution. “O₂ out” refers to the oxygen in the perfusate after it passes through the heart. “Water” indicates data recorded from air-saturated water, to check oxygraphy. “During injection” indicates data that were recorded to check for mixing and possible associated changes in oxygenation. Dorzolamide (1 mM) was added to the solution twice over the course of the measurements.

FIG. 32 shows the oxygen consumption by hearts in control solutions and solutions containing 1 mM acetazolamide or dorzolamide, demonstrating increased oxygen consumption resulting from the presence of each carbonic anhydrase inhibitor.

### DETAILED DESCRIPTION

### Compositions and Uses of Carbonic Anhydrase II (CAII)

It was previously observed that some preparations of carbonic anhydrase II (CAII) solutions had nitrite reductase activity (e.g., the ability to catalyze the conversion of nitrite, NO₂⁻, into nitric oxide, NO) and some did not, though the reason for this distinction remained unclear. For example, Aamand et al. (Am. J. Physiol. Heart Circ. Physiol., 2009; 297:H2068) noted that bovine CAII could generate NO from NO2⁻, but did not identify the mechanism by which this NO generation was happening. Conversely, Andring et al. (Free Radic. Biol. Med. 2018; 117:1-5) demonstrated that in their preparations, CAII did not catalyze the generation of NO.

This disclosure is based, at least in part, on the discovery that when prepared according to the methods described herein and in particular compositions (also described herein), CAII demonstrates nitrite reductase activity (e.g., producing NO from NO2⁻). In some embodiments, CAII bound to copper (e.g., copper ions) at specific sites is capable of catalyzing the reduction of nitrite into nitric oxide. In some embodiments, CAII having nitrite reductase activity according to the present disclosure is free of zinc (e.g., zinc ions).

Accordingly, provided herein is a composition comprising carbonic anhydrase II (CAII) and copper, wherein the composition has nitrite reductase activity. Also provided herein are methods of making said compositions, and methods of using said compositions to treat disease (e.g., heart disease).

Carbonic anhydrase II (CAII) is one of twelve enzymatically active isoforms of carbonic anhydrase produced in humans. CAI and CAII are abundant in most cells, with particularly relevant levels in red blood cells. CAII is a metalloenzyme whose most well-characterized activity is the catalysis of the reversible hydration of CO₂ into HCO₃⁻, and it further catalyzes similar reactions of water with classes of other molecules such as esters, sulfates and phosphates, demonstrating esterase, sulfatase and phosphatase activity, respectively. The hydration and dehydration of CO₂ and HCO₃⁻, respectively, are of particular importance physiologically, as they help to regulate pH and gas homeostasis throughout the body.

### CAII Compositions Having Nitrite Reductase Activity

In some aspects, provided herein is a composition comprising carbonic anhydrase II (CAII) and copper, wherein the composition has nitrite reductase activity.

In some embodiments, a composition of CAII having nitrite reductase activity comprises CAII that is mammalian (e.g., human, bovine, canine, or murine). In some embodiments, CAII as provided in a composition herein is human. In some embodiments, a composition of CAII having nitrite reductase activity comprises human CAII comprising or consisting of an amino acid sequence of SEQ ID NO:1. In some embodiments, a composition of CAII having nitrite reductase activity comprises CAII comprising or consisting of an amino acid sequence of SEQ ID NO:1 or a functional fragment thereof. In some embodiments a functional fragment of CAII has nitrite reductase activity. In some embodiments, a composition of CAII having nitrite reductase activity comprises CAII having amino acid sequence of SEQ ID NO:1 or a functional fragment thereof. Also provided herein is a nucleic acid encoding CAII, comprising or consisting of a nucleic acid sequence of SEQ ID NO:2 or a nucleic acid sequence which upon translation would encode a polypeptide comprising or consisting of the amino acid sequence of SEQ ID NO:1.

It should be noted in some embodiments, the amino acid numbering in the amino acid sequence of CAII omits the number 126. Accordingly, in some embodiments, a protein sequence of CAII having only 260 amino acids will have numbering up to 261.

In some embodiments, a composition having nitrite reductase activity comprises CAII in a concentration between 0.01 mg/mL and 20 mg/mL. In some embodiments, a composition having nitrite reductase activity comprises CAII in a concentration between 0.01 mg/mL and 15 mg/mL, between 0.01 mg/mL and 14 mg/mL, between 0.01 mg/mL and 13 mg/mL, between 0.01 mg/mL and 12 mg/mL, between 0.01 mg/mL and 11 mg/mL, between 0.01 mg/mL and 10 mg/mL, between 0.01 mg/mL and 9 mg/mL, between 0.01 mg/mL and 8 mg/mL, between 0.01 mg/mL and 7 mg/mL, between 0.01 mg/mL and 6 mg/mL, between 0.01 mg/mL and 5 mg/mL, between 0.01 mg/mL and 4 mg/mL, between 0.01 mg/mL and 3 mg/mL, between 0.01 mg/mL and 2 mg/mL, between 0.01 mg/mL and 1 mg/mL, between 0.1 mg/mL and 20 mg/mL, between 0.1 mg/mL and 15 mg/mL, between 0.1 mg/mL and 14 mg/mL, between 0.1 mg/mL and 13 mg/mL, between 0.1 mg/mL and 12 mg/mL, between 0.1 mg/mL and 11 mg/mL, between 0.1 mg/mL and 10 mg/mL, between 0.1 mg/mL and 9 mg/mL, between 0.1 mg/mL and 8 mg/mL, between 0.1 mg/mL and 7 mg/mL, between 0.1 mg/mL and 6 mg/mL, between 0.1 mg/mL and 5 mg/mL, between 0.1 mg/mL and 4 mg/mL, between 0.1 mg/mL and 3 mg/mL, between 0.1 mg/mL and 2 mg/mL, between 0.1 mg/mL and 1 mg/mL, or any range or combination thereof. In some embodiments, a composition having nitrite reductase activity comprises CAII in a concentration of at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1 mg/mL, at least 1.5 mg/mL, at least 2 mg/mL, at least 2.5 mg/mL, at least 3 mg/mL, at least 3.5 mg/mL, at least 4 mg/mL, at least 5 mg/mL, at least 6 mg/mL, at least 7 mg/mL, at least 8 mg/mL, at least 9 mg/mL, at least 10 mg/mL, at least 15 mg/mL or at least 20 mg/mL. In some embodiments, a composition having nitrite reductase activity comprises CAII in a concentration of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 1.5 mg/mL, about 2 mg/mL, about 2.5 mg/mL, about 3 mg/mL, about 3.5 mg/mL, about 4 mg/mL, about 5 mg/mL, about 6 mg/mL, about 7 mg/mL, about 8 mg/mL, about 9 mg/mL or about 10 mg/mL. In some embodiments, a composition having nitrite reductase activity comprises CAII in a concentration of about 0.1 mg/mL, 1 mg/mL or 10 mg/mL.

In some embodiments, a composition having nitrite reductase activity comprises copper in a concentration between 0.01 μM and 100 μM. In some embodiments, a composition having nitrite reductase activity comprises copper in a concentration between 0.1 μM and 10 μM, between 0.1 μM and 9 μM, between 0.1 μM and 8 μM, between 0.1 μM and 7 μM, between 0.1 μM and 6 μM, between 0.1 μM and 5 μM, between 0.1 μM and 4 μM, between 0.1 μM and 3 μM, between 0.1 μM and 2 μM, between 0.1 μM and 1 μM, or any range or combination thereof. In some embodiments, a composition having nitrite reductase activity comprises CAII in a concentration of at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.5 μM, at least 2 μM, at least 2.5 μM, at least 3 μM, at least 3.5 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, or at least 100 μM. In some embodiments, a composition having nitrite reductase activity comprises CAII in a concentration of less than 0.1 μM, less than 0.2 μM, less than 0.3 μM, less than 0.4 μM, less than 0.5 μM, less than 0.6 μM, less than 0.7 μM, less than 0.8 μM, less than 0.9 μM, less than 1 μM, less than 1.5 μM, less than 2 μM, less than 2.5 μM, less than 3 μM, less than 3.5 μM, less than 4 μM, less than 5 μM, less than 6 μM, less than 7 μM, less than 8 μM, less than 9 μM, less than 10 μM, less than 20 μM, less than 30 μM, less than 40 μM, less than 50 μM, less than 60 μM, less than 70 μM, less than 80 μM, less than 90 μM, or less than 100 μM. In some embodiments, a composition having nitrite reductase activity comprises copper in a concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 1.5 μM, about 2 μM, about 2.5 μM, about 3 μM, about 3.5 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, or about 100 μM. In some embodiments, a composition having nitrite reductase activity comprises CAII in a concentration of about 1 μM.

In some embodiments, a composition of CAII having nitrite reductase activity as provided herein comprises copper.

Copper (Cu) is a chemical element with atomic number 29, which has numerous roles in biochemistry including in biological electron transport and oxygen transport. It is an essential cofactor for many enzymes and proteins and plays a role in the mobilization of tissue iron stores. In humans, the adult body contains between 1.4 and 2.1 mg of copper per kilogram of body weight, which is found mostly in the muscle and the liver. The normal range for total copper in the blood is from 70 to 140 micrograms per deciliter, which includes the amount of copper bound to ceruloplasmin. Non-ceruloplasmin-bound copper in the blood is normally in the range of about 10 to about 15 micrograms per deciliter, including a majority of which is loosely bound to albumin. Copper is most commonly found in Cu(I) and Cu(II) oxidation states, having +1 and +2 charge, respectively, and the Cu(II) ion is more stable in aqueous solutions. Compounds containing Cu(II) exhibit a wide range of stereochemistries with four, five, and six coordination compounds predominating. Compositions disclosed herein (e.g., compositions comprising CAII, compositions comprising CAII and copper, compositions having nitrite reductase activity, and/or compositions administered to subjects) in some embodiments comprise copper in a form disclosed herein (e.g., in a Cu(I) or Cu(II) oxidation state or in a copper salt form).

Copper useful in the compositions and methods disclosed herein (e.g., compositions comprising CAII, methods of preparing compositions comprising CAII, and methods of administering CAII) include salt forms of copper, including Cu(I) salts and Cu(II) salts. Examples of Cu(I) salts include but are not limited to copper(I) oxide, copper(I) chloride, copper(I) iodide, copper(I) cyanide, copper(I) thiocyanate, copper(I) sulfate, copper(I) sulfide, copper(I) acetylide, copper(I) bromide, copper(I) fluoride, copper(I) hydroxide, copper(I) hydride, copper(I) nitrate, copper(I) phosphide, copper(I) thiophene-2-carboxylate, and copper(I) t-butoxide. Examples of Cu(II) salts include but are not limited to copper(II) sulfate, copper(II) chloride, copper(II) hydroxide, copper(II) nitrate, copper(II) oxide, copper(II) acetate, copper(II) fluoride, copper(II) bromide, copper(II) carbonate, copper(II) carbonate hydroxide, copper(II) chlorate, copper(II) arsenate, copper(II) azide, copper(II) acetylacetonate, copper(II) aspirinate, copper(II) cyanurate, copper(II) glycinate, copper(II) phosphate, copper(II) perchlorate, copper(II) selenite, copper(II) sulfide, copper(II) thiocyanate, copper(II) triflate, copper(II) tetrafluoroborate, copper(II) acetate triarsenite, copper(II) benzoate, copper(II) arsenite, copper(II) chromite, copper(II) gluconate, copper(II) peroxide, and copper(II) usnate.

Proteins having copper ions as prosthetic groups, known as copper proteins, are found throughout aerobic organisms. These proteins contain copper centers that can be classified into one of six categories: type I copper centers (T1Cu), type II copper centers (T2Cu), type III copper centers (T3Cu), copper A centers (Cu_{A}), copper B centers (Cu_{B}) and copper Z centers (Cu_{Z}). Each of these copper centers involve different coordination modes and geometries, with binding facilitated by different combinations of amino acids of the copper proteins.

In some embodiments of any one of the CAII compositions having nitrite reductase activity as provided here, copper is bound to carbonic anhydrase (e.g., CAII). In some embodiments, copper is bound to CAII through Cu_{A}, Cu_{B}, or both Cu_{A} and Cu_{B} centers. In some embodiments, CAII comprises a Cu_{B} center comprising His94, His96 and His119 of CAII. In some embodiments, CAII comprises a Cu_{A} center comprising His4, His3 and Ser2 of CAII. In some embodiments, the Cu_{B} center of CAII is bound to a copper atom. In some embodiments, His94, His96 and His119 of CAII are bound to a copper atom. In some embodiments, the Cu_{A} center of CAII is bound to a copper atom. In some embodiments, His4, His3 and Ser2 of CAII are bound to a copper atom. In some embodiments His94, His96 and His119 of CAII are bound to a copper atom and His4, His3 and Ser2 of CAII are bound to a copper atom.

In some embodiments of any one of the CAII compositions having nitrite reductase activity as provided here, the composition comprises a plurality of CAII molecules, wherein at least 10% (e.g., at least 10%, at least 20%, at least 30%, at least 40%, at least 50%, at least 60%, at least 70%, at least 80%, at least 90%, at least 95%, or at least 99%) of the plurality of CAII molecules bind a copper atom through His94, His96, and His119 of the CAII. In some embodiments, the composition comprises a plurality of CAII molecules, wherein at least 10%, (e.g., at least 10%, at least 20%, at least 30%, at least 40%, at least 50%, at least 60%, at least 70%, at least 80%, at least 90%, at least 95%, or at least 99%) of the plurality of CAII molecules bind a copper atom through His4, His3, and Ser2 of the CAII. In some embodiments, the composition comprises a plurality of CAII molecules, wherein at least 10%, (e.g., at least 10%, at least 20%, at least 30%, at least 40%, at least 50%, at least 60%, at least 70%, at least 80%, at least 90%, at least 95%, or at least 99%) of the plurality of CAII molecules bind a first copper atom through His94, His96, and His119 of the CAII, and a second copper atom through His4, His3, and Ser2 of the CAII.

In some embodiments, a CAII composition having nitrite reductase activity comprises CAII and copper in a molar ratio between 0.01:1 and 10:1 of CAII to copper. In some embodiments, a CAII composition having nitrite reductase activity comprises CAII and copper in a molar ratio between 0.01:1 and 9:1, between 0.01:1 and 8:1, between 0.01:1 and 7:1, between 0.01:1 and 6:1, between 0.01:1 and 5:1, between 0.01:1 and 4:1, between 0.01:1 and 3:1, between 0.01:1 and 2:1, between 0.01:1 and 1:1, between 0.05:1 and 1:1, between 0.06:1 and 1:1, between 0.07:1 and 1:1, between 0.08:1 and 1:1, between 0.09:1 and 1:1, between 0.1:1 and 1:1 of CAII to copper, or any range or combination thereof. In some embodiments, a CAII composition having nitrite reductase activity comprises CAII and copper in a molar ratio between 0.1:1 and 1:1 of CAII to copper.

In some embodiments, a composition comprising CAII and Cu as provided herein comprises a certain fraction of Cu that is bound to CAII, while the rest is free Cu that not bound to CAII. In some embodiments, the fraction of Cu that is bound to CAII in any one the CAII compositions provided herein is 0.01-99% (e.g., 0.01-0.1, 0.1-1, 1-10, 10-20, 10-50, 20-40, 20-60, 40-60, 50-99, 60-99, 60-80, 80-90, 90-95, 80-99, or 99-99.9%). In some embodiments, at least 5% (e.g., at least 5, at least 10, at least 15, at least 20, at least 25, at least 30, at least 40, at least 50, at least 60, at least 70, at least 80, at least 90, or at least 99%) of the Cu in a composition of CAII is bound to CAII.

As disclosed herein a composition “having nitrite reductase activity” refers to a composition capable of reducing nitrite (NO₂⁻) to a detectable degree or at a detectable rate, such as a composition having catalytic activity which facilitates the reaction of NO₂⁻ into more reduced form. For example, a composition having nitrite reductase activity may facilitate the production of NO from NO₂⁻, at a rate or to such a degree that the rate of production of NO can be measured and/or the rate of consumption of NO₂⁻ can be measured. A composition having nitrite reductase activity in some embodiments has nitrite reductase activity of at least 0.5 nmol nitrite reduced per minute, or between 0.5 and 1000 nmol nitrite reduced per minute. In some embodiments a composition having nitrite reductase activity has nitrite reductase activity between 0.5 and 100, between 0.5 and 90, between 0.5 and 80, between 0.5 and 70, between 0.5 and 60, between 0.5 and 50, between 0.5 and 40, between 0.5 and 30, between 0.5 and 20, between 0.5 and 10, between 0.5 and 9, between 0.5 and 8, between 0.5 and 7, between 0.5 and 6, between 0.5 and 5, between 0.5 and 4, between 0.5 and 3, between 0.5 and 2, between 0.5 and 1 nmol nitrite reduced per minute, or any range or combination thereof. Methods of detecting or measuring nitrite reductase activity are discussed below.

In some embodiments provided herein is a composition comprising carbonic anhydrase II (CAII) and copper that has detectable nitrite reductase activity. In some embodiments provided herein is a composition comprising carbonic anhydrase II (CAII) (e.g., a composition comprising CAII and copper) that has nitrite reductase activity of at least 0.5 nmol nitrite reduced per minute, or between 0.5 and 1000 nmol nitrite reduced per minute. In some embodiments provided herein is a composition comprising carbonic anhydrase II (CAII) and copper that has nitrite reductase activity between 0.5 and 100, between 0.5 and 90, between 0.5 and 80, between 0.5 and 70, between 0.5 and 60, between 0.5 and 50, between 0.5 and 40, between 0.5 and 30, between 0.5 and 20, between 0.5 and 10, between 0.5 and 9, between 0.5 and 8, between 0.5 and 7, between 0.5 and 6, between 0.5 and 5, between 0.5 and 4, between 0.5 and 3, between 0.5 and 2, between 0.5 and 1 nmol nitrite reduced per minute, or any range or combination thereof. Methods of detecting or measuring nitrite reductase activity are discussed below.

In some embodiments, the pH of any one of the compositions comprising CAII and Cu as described herein has a pH of 7-8 (e.g., 7-8, 7.1-7.9, 7.2-7.8, 7.3-7.7, 7.2-7.8, or 7.4-7.6). In some embodiments, the pH of any one of the compositions comprising CAII and Cu as described herein has a pH of 5.5-6.5 (e.g., 5.5-6.5, 5.5-6, 6-6.5, 5.6-6.4, 5.7-6.3, 5.8-6.2, 5.9-6.1, 5.5, 5.6, 5.7, 5.8, 5.9, 6, 6.1, 6.2, 6.3, 6.4, or 6.5). In some embodiments, the pH of a CAII composition as provided herein is 5-9.

In some embodiments, less than 10% (e.g., less than 10, less than 5, less than 1, less than 0.1, or less than 0.01%) of the CAII in any one of the compositions of CAII provided herein is bound to a metal other than Cu (e.g., Zn). In some embodiments, no more than 10% (e.g., no more than 10, no more than 5, no more than 1, no more than 0.1, or no more than 0.01%) of the CAII in any one of the compositions of CAII provided herein is bound to a metal other than Cu (e.g., Zn). In some embodiments, a composition of CAII having nitrite reductase activity has less than 0.01M (e.g., less than 0.01M, less than 0.001M, less than 0.001M, less than 0.1 mM, less than 0.01 mM, less than 1 μM, less than 1 nM, or less than 0.1 nM) of metal other than Cu (Zn).

### Pharmaceutically Acceptable Carriers

In some embodiments, any one of the compositions provided here comprises a pharmaceutically acceptable carrier. The term “carrier” refers to a diluent, adjuvant, excipient, or vehicle with which the therapeutic agent (e.g., a composition comprising CAII and Cu) is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of composition comprising CAII and Cu to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

In some embodiments, a pharmaceutically acceptable carrier for carbonic anhydrase II (CAII) or a pharmaceutical composition comprising CAII contains 50 mM or about 50 mM Tris-HCl. In some embodiments, a pharmaceutically acceptable carrier for CAII has a pH of 7.8 or about 7.8. In some embodiments, a pharmaceutically acceptable carrier for CAII or a pharmaceutical composition comprising CAII contains 1 to 1000 mM Tris-HCl (e.g., 1 to 900 mM, 1 to 800 mM, 1 to 700 mM, 1 to 600 mM, 1 to 500 mM, 1 to 400 mM, 1 to 300 mM, 1 to 200 mM, 1 to 100 mM, 1 to 50 mM, 1 to 25 mM, 25 to 1000 mM, 25 to 925 mM, 25 to 900 mM, 25 to 825 mM, 25 to 800 mM, 25 to 725 mM, 25 to 700 mM, 25 to 625 mM, 25 to 600 mM, 25 to 525 mM, 25 to 250 mM, 25 to 425 mM, 25 to 400 mM, 25 to 325 mM, 25 to 300 mM, 25 to 225 mM, 25 to 200 mM, 25 to 125 mM, 25 to 100 mM, 25 to 50 mM, 50 to 1000 mM, 50 to 950 mM, 50 to 900 mM, 50 to 850 mM, 50 to 800 mM, 50 to 750 mM, 50 to 700 mM, 50 to 650 mM, 50 to 600 mM, 50 to 550 mM, 50 to 500 mM, 50 to 450 mM, 50 to 400 mM, 50 to 350 mM, 50 to 300 mM, 50 to 250 mM, 50 to 200 mM, 50 to 150 mM, 50 to 100 mM, 100 to 950 mM, 100 to 900 mM, 100 to 850 mM, 100 to 800 mM, 100 to 750 mM, 100 to 700 mM, 100 to 650 mM, 100 to 600 mM, 100 to 550 mM, 100 to 500 mM, 100 to 450 mM, 100 to 400 mM, 100 to 350 mM, 100 to 300 mM, 100 to 250 mM, 100 to 200 mM, 100 to 150 mM, 150 to 950 mM, 150 to 900 mM, 150 to 850 mM, 150 to 800 mM, 150 to 750 mM, 150 to 700 mM, 150 to 650 mM, 150 to 600 mM, 150 to 550 mM, 150 to 500 mM, 150 to 450 mM, 150 to 400 mM, 150 to 350 mM, 150 to 300 mM, 150 to 250 mM, 150 to 200 mM, 200 to 950 mM, 200 to 900 mM, 200 to 850 mM, 200 to 800 mM, 200 to 750 mM, 200 to 700 mM, 200 to 650 mM, 200 to 600 mM, 200 to 550 mM, 200 to 500 mM, 200 to 450 mM, 200 to 400 mM, 200 to 350 mM, 200 to 300 mM, 200 to 250 mM, or any range or combination thereof.

In some embodiments, a pharmaceutically acceptable carrier for CAII or a pharmaceutical composition comprising CAII has a pH of 7-9 (e.g., 7-8, 7.1-7.9, 7.2-7.8, 7.3-7.7, 7.2-7.8, 7.4-7.6, 8-9, 8.1-8.9, 8.2-8.8, 8.3-8.7, 8.2-8.8, 8.4-8.6, 7.5-8.5, 7.6-8.5, 7.7-8.5, 7.8-8.5, 7.9-8.5, 8.0-8.5, 7.5-8.4, 7.5-8.3, 7.5-8.2, 7.5-8.1, 7.5-8.0, 7.5-7.9. 7.5-7.8, 7.5-7.7, 7.5-7.6, 7.6-8.3, 7.6-8.2, 7.6-8.1, 7.6-8.0, 7.6-7.9, 7.6-7.8, 7.6-7.7, 7.7-8.3, 7.7-8.2, 7.7-8.1, 7.7-8.0, 7.7-7.9, 7.7-7.8, 7.8-8.3, 7.8-8.2, 7.8-8.1, 7.8-8.1, 7.8-8.0, 7.8-7.9). In some embodiments, a pharmaceutically acceptable carrier for CAII or a pharmaceutical composition comprising CAII has a pH of 7.5 or about 7.5. In some embodiments, a pharmaceutically acceptable carrier for CAII or a pharmaceutical composition comprising CAII has a pH of 7.6 or about 7.6. In some embodiments, a pharmaceutically acceptable carrier for CAII or a pharmaceutical composition comprising CAII has a pH of 7.7 or about 7.7. In some embodiments, a pharmaceutically acceptable carrier for CAII or a pharmaceutical composition comprising CAII has a pH of 7.8 or about 7.8. In some embodiments, a pharmaceutically acceptable carrier for CAII or a pharmaceutical composition comprising CAII has a pH of 7.9 or about 7.9. In some embodiments, a pharmaceutically acceptable carrier for CAII or a pharmaceutical composition comprising CAII has a pH of 8.0 or about 8.0. In some embodiments, a pharmaceutically acceptable carrier for CAII or a pharmaceutical composition comprising CAII has a pH of 8.1 or about 8.1. In some embodiments, a pharmaceutically acceptable carrier for CAII or a pharmaceutical composition comprising CAII has a pH of 8.2 or about 8.2.

In some embodiments, a pharmaceutically acceptable carrier for CAII or a pharmaceutical composition comprising CAII contains 50 mM or about 50 mM Tris-HCl and has a pH of 7.6 or about 7.6. In some embodiments, a pharmaceutically acceptable carrier for CAII or a pharmaceutical composition comprising CAII contains 50 mM or about 50 mM Tris-HCl and has a pH of 7.7 or about 7.7. In some embodiments, a pharmaceutically acceptable carrier for CAII or a pharmaceutical composition comprising CAII contains 50 mM or about 50 mM Tris-HCl and has a pH of 7.8 or about 7.8. In some embodiments, a pharmaceutically acceptable carrier for CAII or a pharmaceutical composition comprising CAII contains 50 mM or about 50 mM Tris-HCl and has a pH of 7.9 or about 7.9. In some embodiments, a pharmaceutically acceptable carrier for CAII or a pharmaceutical composition comprising CAII contains 50 mM or about 50 mM Tris-HCl and has a pH of 8.0 or about 8.0. In some embodiments, a pharmaceutically acceptable carrier for CAII or a pharmaceutical composition comprising CAII contains 50 mM or about 50 mM Tris-HCl and has a pH of 8.1 or about 8.1.

In some embodiments, a pharmaceutically acceptable carrier for CAII or a pharmaceutical composition comprising CAII contains no more than 0.1% (w/v) of metal chelators (e.g., ethylenediaminetetraacetic acid (EDTA), pyridine-2,6-dicarboxylic acid (DPA)). In some embodiments, a pharmaceutically acceptable carrier for CAII or a pharmaceutical composition comprising CAII contains no more than 0.09%, no more than 0.08%, no more than 0.07%, no more than 0.06%, no more than 0.05%, no more than 0.04%, no more than 0.03%, no more than 0.02%, no more than 0.01%, no more than 0.009%, no more than 0.008%, no more than 0.007%, no more than 0.006%, no more than 0.005%, no more than 0.004%, no more than 0.003%, no more than 0.002%, no more than 0.001%, no more than 0.0009%, no more than 0.0008%, no more than 0.0007%, no more than 0.0006%, no more than 0.0005%, no more than 0.0004%, no more than 0.0003%, no more than 0.0002%, no more than 0.0001% or less of metal chelators (e.g., EDTA, DPA). In some embodiments, a pharmaceutically acceptable carrier for CAII or a pharmaceutical composition comprising CAII contains no measurable amount of metal chelator (e.g., EDTA, DPA). In some embodiments, a pharmaceutically acceptable carrier for CAII or a pharmaceutical composition comprising CAII contains no metal chelator (e.g., EDTA, DPA).

In some embodiments, a pharmaceutically acceptable carrier for CAII or a pharmaceutical composition comprising CAII contains no more than 0.1% (w/v) of reducing agents (e.g., dithiothreitol (DTT)). In some embodiments, a pharmaceutically acceptable carrier for CAII or a pharmaceutical composition comprising CAII contains no more than 0.09%, no more than 0.08%, no more than 0.07%, no more than 0.06%, no more than 0.05%, no more than 0.04%, no more than 0.03%, no more than 0.02%, no more than 0.01%, no more than 0.009%, no more than 0.008%, no more than 0.007%, no more than 0.006%, no more than 0.005%, no more than 0.004%, no more than 0.003%, no more than 0.002%, no more than 0.001%, no more than 0.0009%, no more than 0.0008%, no more than 0.0007%, no more than 0.0006%, no more than 0.0005%, no more than 0.0004%, no more than 0.0003%, no more than 0.0002%, no more than 0.0001% or less of reducing agents (e.g., DTT). In some embodiments, a pharmaceutically acceptable carrier for CAII or a pharmaceutical composition comprising CAII contains no measurable amount of reducing agents (e.g., DTT). In some embodiments, a pharmaceutically acceptable carrier for CAII or a pharmaceutical composition comprising CAII contains no reducing agents (e.g., DTT).

Typically, such compositions may contain at least about 0.1% of the therapeutic agent (e.g., a composition comprising CAII and Cu) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., composition comprising CAII and Cu) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Making CAII

Provided herein are methods of making CAII compositions that have nitrite reductase activity. In some embodiments, a method of making a composition comprising CAII that has nitrite reductase activity, comprises purifying (or isolating) CAII from a biological source of CAII (e.g., a blood sample or culture of bacteria), chelating metal ions from the purified CAII, and incubating the purified CAII from which metal ions are chelated with copper. In some embodiments, a method of making a composition comprising CAII that has nitrite reductase activity, comprises purifying (or isolating) CAII from a biological source of CAII (e.g., a natural source such as blood, or a synthetic source such as produced by cultured bacteria in a lysate), chelating metal ions from the purified CAII, and incubating the purified CAII from which metal ions are chelated with copper. In some embodiments, purified CAII from which metal ions have been chelated is incubated with Cu at a molar ratio of 0.1:1 to 1:1 (e.g., 0.1:1, 0.2:1, 0.3:1, 0.4:1, 0.5:1, 0.6:1, 0.7:1, 0.8:1, 0.9:1, or 1:1) of CAII to copper. In some embodiments the ratio of CAII to copper is less than 0.1:1 or even less than 0.01:1 (e.g., 0.01:1 or 0.001:1).

In some embodiments, CAII is purified from or isolated from a biological sample (e.g., a blood sample or a bacterial culture). In some embodiments, “purification” or “isolation” means retrieving CAII from a biological source. In some embodiments, purified CAII is CAII in which certain components (e.g., salts, proteins, peptides, or metals) which were present in the biological source are lower in quantity or concentration in the purified CAII. In some embodiments, purification of CAII can be achieved through a method which includes affinity chromatography, size exclusion chromatography, gel permeation chromatography, ion exchange chromatography, hydrophobic interaction chromatography, free-flow electrophoresis, high performance liquid chromatography (HPLC), spin filtration, dialysis, centrifugation, precipitation, gel electrophoresis, or any combination thereof. In some embodiments CAII is purified using a p-aminomethyl-benzenesulfonamide affinity column. In some embodiments CAII can be purified from a blood sample or culture of bacteria through a method which includes buffer exchange. It should be understood that CAII can be purified from other systems, including organic and synthetic systems. Non-limiting examples of systems from which CAII can be purified include prokaryotic cell cultures, eukarytic cell cultures and in vitro translation systems.

According to some aspects, the present disclosure provides purified CAII in which metal ions have been chelated. Chelating metal ions from purified CAII can be accomplished by incubating the CAII composition with one or more chelators. Non-limiting examples of methods to chelate metal ions from purified CAII include incubating the purified CAII with ethylenediaminetetraacetic acid (EDTA), ethylenediamine, methylamine, pyridine-2,6-dicarboxylic acid (DPA) or a combination thereof. In some embodiments, chelating metal ions from the purified CAII is done by incubating the purified CAII with a chelating agents (e.g., pyridine-2,6-dicarboxylic acid (DPA)). In some embodiments CAII is incubated with a chelating agent (e.g., pyridine-2,6-dicarboxylic acid (DPA)) at a concentration of between 1 mM and 1M (e.g., between 1 mM and 900 mM, between 1 mM and 800 mM, between 1 mM and 700 mM, between 1 mM and 650 mM, between 1 mM and 600 mM, between 1 mM and 550 mM, between 1 mM and 500 mM, between 5 mM and 550 mM, between 10 mM and 550 mM, between 20 mM and 550 mM, between 30 mM and 550 mM, between 40 mM and 550 mM, between 50 mM and 550 mM, between 100 mM and 550 mM, between 150 mM and 550 mM, between 200 mM and 550 mM, between 250 mM and 550 mM, between 300 mM and 550 mM, between 350 mM and 550 mM, between 400 mM and 550 mM, between 450 mM and 550 mM, or any range or combination thereof). In some embodiments, CAII is incubated with a chelating agent (e.g., pyridine-2,6-dicarboxylic acid (DPA)) at a concentration of 500 mM or about 500 mM.

In some embodiments, purified CAII is incubated with a metal chelator so that less than 10% (e.g., less than 10%, less than 5%, less than 1%, less than 0.1%, or less than 0.01%) of the CAII in any one of the compositions of CAII provided herein is bound to a metal (e.g., a metal other than Cu, such as Zn). In some embodiments, a composition of CAII having nitrite reductase activity has less than 0.01M (e.g., less than 0.01M, less than 0.001M, less than 0.001M, less than 0.1 mM, less than 0.01 mM, less than 1 μM, less than 1 nM, or less than 0.1 nM) of metal (e.g., a metal other than Cu, such as Zn).

In some embodiments, a method of making a composition comprising CAII that has nitrite reductase activity, comprises incubating purified CAII from which metal ions are chelated with copper. In some embodiments, cuprous forms of copper are used. In some embodiments, cupric forms of copper are used. Copper may be in different forms, e.g., oxides, sulfides, or halides.

Contemplated herein is also a composition of a gene delivery vector (e.g., adeno-associated viral vector, retrovirus, adenovirus, or oligonucleotides in liposomal delivery systems) comprising nucleic acid encoding CAII that can be delivered to a subject.

### Measurement of Nitrite Reductase Activity

Nitrite reductase activity can be measured by many methods, including methods which measure the production of reduced products (e.g., NO) and methods which measure the depletion of reactants (e.g., NO₂⁻). Non-limiting examples of such methods include spectrophotometric methods using methyl viologen (MV), diquat, phenosafranine (PS) or anthraquinone-2-sulphonate (AQS) as electron sources; protein film voltammetry; gas chromatography-mass spectrometry (GC-MS) measurement of NO₂⁻; measurement of NO production via NO-sensitive electrode; and measurement of NO production via membrane inlet mass spectrometry (MINIS). See, e.g., Ramirez et al., Biochim. Biophys. Acta 1966 118:58-71; Silveira, et al., Bioinorg. Chem. Appl. 2010 pii: 634597; Hanff et al., Anal. Biochem. 550: 132-136, 2018; the contents of each of which are incorporated herein by reference in their entireties.

### Methods of Administering CAII to a Subject

Any one of the CAII compositions having nitrite reductase activity are useful to treat conditions that can be relieved by causing vasodilation. Accordingly, provided herein is a method comprising administering to a subject any one of the CAII compositions nitrite reductase activity.

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, a subject to which a CAII comprising composition (e.g., a composition comprising CAII and copper) is administered is a subject that suffers from or is at risk of suffering from a condition that can be relieved by causing vasodilation. Non-limiting examples of conditions that can be relieved by vasodilation include hypertension, pulmonary hypertension, a heart condition (e.g., heart failure, angina, coronary artery disease, or myocardial infarction), erectile dysfunction, or muscular atrophy. Hypertension may be primary hypertension or secondary hypertension, wherein the secondary hypertension is secondary to eclampsia, preeclampsia, renovascular disease or renal disease, sleep apnea, or endocrine abnormalities. In some embodiments, the condition that can be relieved by causing vasodilation is a cardiovascular condition. In some embodiments, the cardiovascular condition is hypertension (e.g., high blood pressure), heart failure (e.g., acute heart failure, congestive heart failure, chronic heart failure), ischemic heart disease, pulmonary hypertension, pulmonary arterial hypertension (e.g., idiopathic pulmonary arterial hypertension or hereditary pulmonary arterial hypertension), chronic thromboembolic pulmonary hypertension, pulmonary edema, angina (e.g., angina pectoris), unstable angina, chronic stable angina, coronary artery disease, myocardial infarction (e.g., acute myocardial infarction), cardiomyopathy, erectile dysfunction, muscle atrophy, preeclampsia or eclampsia. In some embodiments, the condition that can be relieved by causing vasodilation is an acute coronary syndrome. In some embodiments, the condition that can be relieved by causing vasodilation is myocardial infarction. In some embodiments, the condition that can be relieved by causing vasodilation is hypertension. In some embodiments, the condition that can be relieved by causing vasodilation is peripheral arterial disease or peripheral vascular disease. In some embodiments, the condition is Raynaud's disease or Raynaud's phenomenon. In some embodiments, the condition that can be relieved by causing vasodilation is dyspnea. In some embodiments, the condition that can be relieved by causing vasodilation is scleroderma. In some embodiments, the condition is a risk of a cardiovascular condition, such as a risk of heart attack or stroke, or any of the conditions described above. In some embodiments, the condition is diabetic neuropathy. In some embodiments, the condition is pheochromocytoma or hyperadrenergic state. In some embodiments, a subject to which a CAII comprising composition (e.g., a composition comprising CAII and copper) is administered is a subject in need of vasodilation. In some embodiments, a subject in need of vasodilation is a subject undergoing radiation therapy. In some embodiments, a subject in need of vasodilation is a subject being treated with certain drugs, including but not limited to cancer therapeutics. In some embodiments, a subject in need of vasodilation is a subject undergoing surgery.

In some embodiments, “administering” or “administration,” for example, in the context of CAII compositions means providing a material (e.g., a CAII composition) to a subject in a manner that is pharmacologically useful. In some embodiments, a composition comprising CAII (e.g., a composition comprising CAII and copper) is administered to a subject enterally. In some embodiments, an enteral administration of the composition is oral. In some embodiments, a composition comprising CAII (e.g., a composition comprising CAII and copper) is administered to the subject parenterally. In some embodiments, a composition comprising CAII (e.g., a composition comprising CAII and copper) is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a composition comprising CAII (e.g., a composition comprising CAII and copper) is administered to the subject by injection into the hepatic artery or portal vein.

To “treat” a disease as the term is used herein in the context of CAII compositions, means to reduce the frequency or severity of at least one sign or symptom of a disease or disorder experienced by a subject. The compositions described above (e.g., compositions comprising CAII, such as compositions comprising CAII and copper) or elsewhere herein are typically administered to a subject in an effective amount, that is, an amount capable of producing a desirable result. The desirable result will depend upon the active agent being administered. For example, an effective amount of a composition comprising CAII (e.g., a composition comprising CAII and copper) may be an amount of the composition that is capable of increasing nitrite reductase activity and/or increasing NO levels. A therapeutically acceptable amount may be an amount that is capable of treating a disease or condition, e.g., a disease or condition that that can be relieved by causing vasodilation, such as a condition described herein, including a heart condition (e.g., myocardial infarction, stroke), hypertension, pulmonary hypertension, erectile dysfunction, Raynaud's phenomenon, or muscular atrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a subject is administered CAII in an amount sufficient to increase the nitrite reductase activity of the subject (e.g., in the blood). Methods of measuring nitrite reductase activity are described herein.

In some embodiments, CAII is administered at a dose of 1 to 10000 mg daily (e.g., 1 to 9000 mg daily, 1 to 8000 mg daily, 1 to 7000 mg daily, 1 to 6000 mg daily, 1 to 5000 mg daily, 1 to 4000 mg daily, 1 to 3000 mg daily, 1 to 2000 mg daily, 1 to 1000 mg daily, 1 to 900 mg daily, 1 to 800 mg daily, 1 to 700 mg daily, 1 to 600 mg daily, 1 to 500 mg daily, 1 to 400 mg daily, 1 to 300 mg daily, 1 to 200 mg daily, 1 to 100 mg daily, 50 to 1000 mg daily, 100 to 1000 mg daily, 150 to 1000 mg daily, 200 to 1000 mg daily, 250 to 1000 mg daily, 300 to 1000 mg daily, 350 to 1000 mg daily, 400 to 1000 mg daily, 450 to 1000 mg daily, 500 to 1000 mg daily, 550 to 1000 mg daily, 600 to 1000 mg daily, 650 to 1000 mg daily, 700 to 1000 mg daily, 750 to 1000 mg daily, 800 to 1000 mg daily, 850 to 1000 mg daily, 900 to 1000 mg daily, 950 to 1000 mg daily, 100 to 950 mg daily, 100 to 900 mg daily, 100 to 850 mg daily, 100 to 800 mg daily, 100 to 750 mg daily, 100 to 700 mg daily, 100 to 650 mg daily, 100 to 600 mg daily, 100 to 550 mg daily, 100 to 500 mg daily, 100 to 450 mg daily, 100 to 400 mg daily, 100 to 350 mg daily, 100 to 300 mg daily, 100 to 250 mg daily, 100 to 200 mg daily, 100 to 150 mg daily, 150 to 950 mg daily, 150 to 900 mg daily, 150 to 850 mg daily, 150 to 800 mg daily, 150 to 750 mg daily, 150 to 700 mg daily, 150 to 650 mg daily, 150 to 600 mg daily, 150 to 550 mg daily, 150 to 500 mg daily, 150 to 450 mg daily, 150 to 400 mg daily, 150 to 350 mg daily, 150 to 300 mg daily, 150 to 250 mg daily, 150 to 200 mg daily, 200 to 950 mg daily, 200 to 900 mg daily, 200 to 850 mg daily, 200 to 800 mg daily, 200 to 750 mg daily, 200 to 700 mg daily, 200 to 650 mg daily, 200 to 600 mg daily, 200 to 550 mg daily, 200 to 500 mg daily, 200 to 450 mg daily, 200 to 400 mg daily, 200 to 350 mg daily, 200 to 300 mg daily, 200 to 250 mg daily, 250 to 950 mg daily, 250 to 900 mg daily, 250 to 850 mg daily, 250 to 800 mg daily, 250 to 750 mg daily, 250 to 700 mg daily, 250 to 650 mg daily, 250 to 600 mg daily, 250 to 550 mg daily, 250 to 500 mg daily, 250 to 450 mg daily, 250 to 400 mg daily, 250 to 350 mg daily, 250 to 300 mg daily, 300 to 950 mg daily, 300 to 900 mg daily, 300 to 850 mg daily, 300 to 800 mg daily, 300 to 750 mg daily, 300 to 700 mg daily, 300 to 650 mg daily, 300 to 600 mg daily, 300 to 550 mg daily, 300 to 500 mg daily, 300 to 450 mg daily, 300 to 400 mg daily, 300 to 350 mg daily, 350 to 950 mg daily, 350 to 900 mg daily, 350 to 850 mg daily, 350 to 800 mg daily, 350 to 750 mg daily, 350 to 700 mg daily, 350 to 650 mg daily, 350 to 600 mg daily, 350 to 550 mg daily, 350 to 500 mg daily, 350 to 450 mg daily, 350 to 400 mg daily, 400 to 950 mg daily, 400 to 900 mg daily, 400 to 850 mg daily, 400 to 800 mg daily, 400 to 750 mg daily, 400 to 700 mg daily, 400 to 650 mg daily, 400 to 600 mg daily, 400 to 550 mg daily, 400 to 500 mg daily, 400 to 450 mg daily, 450 to 950 mg daily, 450 to 900 mg daily, 450 to 850 mg daily, 450 to 800 mg daily, 450 to 750 mg daily, 450 to 700 mg daily, 450 to 650 mg daily, 450 to 600 mg daily, 450 to 550 mg daily, 450 to 500 mg daily, 500 to 950 mg daily, 500 to 900 mg daily, 500 to 850 mg daily, 500 to 800 mg daily, 500 to 750 mg daily, 500 to 700 mg daily, 500 to 650 mg daily, 500 to 600 mg daily, 500 to 550 mg daily, 550 to 950 mg daily, 550 to 900 mg daily, 550 to 850 mg daily, 550 to 800 mg daily, 550 to 750 mg daily, 550 to 700 mg daily, 550 to 650 mg daily, 550 to 600 mg daily, or any range or combination thereof). In some embodiments, the CAII is administered once daily, the CAII is administered twice daily, or the CAII is administered three times daily or more (e.g., the total daily dose is divided between two, three, or more administrations, or the individual dose is administered once, twice, three times or more daily).

In some embodiments, administering a CAII composition increases the amount of copper-bound CAII (Cu-CAII) in a subject (e.g., in blood or tissue of the subject). In some embodiments, the CAII composition is administered at a dose sufficient to increase the amount of Cu-CAII in the subject by 5% or more (e.g., by 10%, by 15%, by 20%, by 25%, by 30%, by 35%, by 40%, by 45%, by 50%, by 60%, by 70%, by 80%, by 90%, by 100%, by 110%, by 120%, by 130%, by 140%, by 150%, by two-fold, by three-fold, by four-fold, by five-fold, by six-fold, by seven-fold, by eight-fold, by nine-fold, by 10-fold, by 11-fold, by 12-fold, by 13-fold, by 14-fold, by 15-fold, by 16-fold, by 17-fold, by 18-fold, by 19-fold, by 20-fold, or more). In some embodiments, the CAII composition is administered at a dose sufficient to increase the amount of Cu-CAII in the subject by 10% or more.

The amount of Cu-CAII in a sample (e.g., a blood or tissue sample from a subject to whom a CAII composition has been or is to be administered) can be measured in a number of ways, such as by purification and graphite furnace atomic absorption spectroscopy (GFAAS, also known as electrothermal atomic absorption spectroscopy). For example, CAII can be purified from a sample by affinity purification (e.g., by using an immunoaffinity column comprising immobilized anti-CAII antibodies). Subsequently, purified CAII can be injected into a GFAAS system, which uses a graphite-coated furnace to vaporize the sample and measure absorption of light at various wavelengths characteristic of the element(s) of interest. Such measurements can then be used to calculate the amount of an element (e.g., copper) in the sample, by applying the Beer-Lambert law or using a standard curve, for example. Rigueira, et al. (Food Chemistry (2016) 211:910-915) provides a representative example use of GFAAS to measure metals (e.g., Cu and Zn) in protein samples. Additional methods by which the amount of Cu-CAII in a sample can be measured include, but are not limited to, purification and X-ray absorption near edge structures (XANES, also known as near edge X-ray absorption fine structure (NEXAFS)).

### Methods of Administering CAII Inhibitors to Activate Nitrite Reductase Activity

The inventors of the present disclosure have found that carbonic anhydrase II (CAII) has nitrite reductase activity when it is bound to copper (e.g., when it is in the Cu-CAII form), and that such nitrite reductase activity can be activated or increased by contacting CAII with, incubating CAII with, and/or placing CAII in the presence of a one or more compounds classically known as CAII inhibitors (e.g., sulfonamide drugs, such as acetazolamide, dichlorphenamide, methazolamide, and dorzolamide). Accordingly, provided herein are methods of administering to a subject one or more compounds classically known as inhibitors of CAII (e.g., sulfonamide compounds, such as acetazolamide, dichlorphenamide, methazolamide, and dorzolamide) to increase nitrite reductase activity in the subject. Provided herein are methods of administering to a subject one or more such compounds in an amount sufficient to increase the nitrite reductase activity in the subject (e.g., in the blood, or of CAII). In some embodiments, the one or more compounds classically known as inhibitors of CAII preferentially inhibit CAII bound to Zn (e.g., Zn-CAII) relative to CAII bound to Cu (e.g., Cu-CAII). In some embodiments, the one or more inhibitors of CAII activate nitrite reductase activity of CAII (e.g., Cu-CAII). In some embodiments, the activation of nitrite reductase activity of CAII (e.g., Cu-CAII) is independent of inhibition activity (e.g., inhibition of carbonic anhydrase activity and/or inhibition of Zn-CAII) and/or is independent of binding to or interaction with CAII (e.g., Zn-CAII).

This represents a particularly unexpected finding because the compounds classically known as carbonic anhydrase inhibitors (e.g., sulfonamide compounds, such as acetazolamide, dichlorphenamide, methazolamide, and dorzolamide), are conventionally understood to function by inhibiting the activity of CAII (e.g., Zn-CAII). By contrast, the present disclosure provides compositions in which such compounds (i.e., classical carbonic anhydrase inhibitors such as sulfonamide compounds) instead activate or enhance the activity of CAII (e.g., Cu-CAII), particularly nitrite reductase activity thereof. In some embodiments, incubating CAII (e.g., Cu-CAII) with, contacting CAII with, or placing CAII in the presence of a classical carbonic anhydrase inhibitor (e.g., a sulfonamide compound disclosed herein) results in an increase in nitrite reductase activity of the CAII. In certain embodiments, this indicates that classical carbonic anhydrase inhibitors (e.g., certain sulfonamides) may have the capacity to activate nitrite reductase activity of CAII (e.g., Cu-CAII) independent of their carbonic anhydrase inhibition functions.

Without being bound by theory, a compound classically known as an inhibitor of CAII (e.g., a sulfonamide compound, such as acetazolamide, dichlorphenamide, methazolamide, or dorzolamide) may serve to activate or enhance nitrite reductase activity of CAII (e.g., Cu-CAII) by acting as an electron donor when associated with (e.g., in contact with or in proximity to) CAII.

As used herein, the term “CAII inhibitor”, “carbonic anhydrase inhibitor” or “inhibitor of carbonic anhydrase” refers to a compound classically known as a carbonic anhydrase (e.g., CAII) inhibitor. Such compounds are described in further detail below.

In some embodiments, a compound classically known as a carbonic anhydrase (e.g., CAII) inhibitor, referred to herein as an inhibitor of carbonic anhydrase, a CAII inhibitor, or a carbonic anhydrase inhibitor, is a sulfonamide-based carbonic anhydrase inhibitor. Non-limiting examples of carbonic anhydrase inhibitors include acetazolamide, methazolamide, ethoxzolamide, dichlorphenamide, dorzolamide, brinzolamide, topiramate, celecoxib, sulpiride, sulthiame, valdecoxib, zonisamide, irosustat, an esterone sulfamate, benzyl-sulfonamide compounds, punicalin, punicalagin, granatin B, gallagyldilactone, casuarinin, pedunculagin and tellimagrandin I. In some embodiments, the inhibitor of carbonic anhydrase is acetazolamide. In some embodiments, the inhibitor of carbonic anhydrase is dorzolamide.

In some embodiments, carbonic anhydrase activity of CAII is reduced by at least 10% (e.g., at least 10%, at least 20%, at least 30%, at least 40%, at least 50%, at least 60%, at least 70%, at least 80%, at least 90%, at least 95%, at least 98%, at least 99%, or at least 99.9%) either when tested in vitro or in vivo. In some embodiments, the carbonic anhydrase activity of CAII is reduced relative to CAII in the absence of a carbonic anhydrase inhibitor. In some embodiments, administering of a CAII inhibitor in a subject results in an activation of nitrite reductase activity, an increase in nitrite reductase activity and/or an increase in NO level in the subject by at least 2% (e.g., at least 2%, at least 5%, at least 10%, at least 20%, at least 30%, at least 40%, at least 50%, at least 60%, at least 70%, at least 80%, at least 90%, at least 95%, at least 98%, at least 99%, at least 100% or more). In some embodiments, administering of a CAII inhibitor in a subject results in an increase in nitrite reductase activity and/or NO level in the subject by at least two-fold (e.g., at least two-fold, at least three-fold, at least four-fold, at least five-fold, at least six-fold, at least seven-fold, at least eight-fold, at least nine-fold, at least 10-fold, at least 15-fold, at least 20-fold, or more). In some embodiments, the nitrite reductase activity and/or NO level is increased relative to a comparable context in which a carbonic anhydrase inhibitor is absent (e.g., in a subject to whom a CAII inhibitor has not been administered, or in the same subject before an drug that might affect the activity is administered). Methods of measuring nitrite reductase activity are discussed above. Methods of measuring NO level include GC-MS, using NO-sensitive electrodes, membrane inlet mass spectrometry (MIMS), and other methods.

In some embodiments, a subject is administered a CAII inhibitor (e.g., a sulfonamide compound disclosed herein) or a CAII inhibitor and a composition comprising CAII (e.g., a composition comprising CAII and copper) to treat a condition that can be relieved by causing vasodilation, such as a condition described herein, including a heart condition (e.g., myocardial infarction, stroke, or Raynaud's phenomenon), hypertension, pulmonary hypertension, erectile dysfunction, or muscular atrophy. As used herein, “treating” can include either therapeutic use or prophylactic use. Administering and treating are discussed further below.

In some embodiments of any one of the methods of administering a CAII inhibitor or a CAII inhibitor and a composition comprising CAII (e.g., a composition comprising CAII and copper) to a subject as provided herein comprises administering a CAII inhibitor or a composition comprising CAII (e.g., a composition comprising CAII and copper) to a subject having or at risk of having a heart condition. Heart conditions include those described herein. Non-limiting examples of a heart condition of a subject who is administered a CAII inhibitor or a CAII inhibitor and a composition comprising CAII (e.g., a composition comprising CAII and copper) according to any one of the methods of administering as disclosed herein are myocardial infarction, stroke, Raynaud's phenomenon, heart failure, angina or coronary artery disease. In some embodiments, a subject to which a CAII inhibitor is administered, has been administered or is going to be administered a composition comprising CAII (e.g., a composition comprising CAII and copper).

In some embodiments, a subject to which a CAII inhibitor or a CAII inhibitor and a composition comprising CAII (e.g., a composition comprising CAII) and copper is administered is a subject that suffers from or is at risk of suffering from a condition that can be relieved by causing vasodilation. Non-limiting examples of conditions that can be relieved by vasodilation include hypertension, pulmonary hypertension, a heart condition (e.g., heart failure, angina, coronary artery disease, or myocardial infarction), erectile dysfunction, or muscular atrophy. Hypertension may be primary hypertension or secondary hypertension, wherein the secondary hypertension is secondary to eclampsia, preeclampsia, renovascular disease or renal disease, sleep apnea, or endocrine abnormalities. In some embodiments, the condition that can be relieved by causing vasodilation is a cardiovascular condition. In some embodiments, the cardiovascular condition is hypertension (e.g., high blood pressure), heart failure (e.g., acute heart failure, congestive heart failure, chronic heart failure), ischemic heart disease, pulmonary hypertension, pulmonary arterial hypertension (e.g., idiopathic pulmonary arterial hypertension or hereditary pulmonary arterial hypertension), chronic thromboembolic pulmonary hypertension, pulmonary edema, angina (e.g., angina pectoris), unstable angina, chronic stable angina, coronary artery disease, myocardial infarction (e.g., acute myocardial infarction), cardiomyopathy, erectile dysfunction, muscle atrophy, preeclampsia or eclampsia. In some embodiments, the condition that can be relieved by causing vasodilation is an acute coronary syndrome. In some embodiments, the condition that can be relieved by causing vasodilation is myocardial infarction. In some embodiments, the condition that can be relieved by causing vasodilation is hypertension. In some embodiments, the condition that can be relieved by causing vasodilation is peripheral arterial disease or peripheral vascular disease. In some embodiments, the condition is Raynaud's disease or Raynaud's phenomenon. In some embodiments, the condition that can be relieved by causing vasodilation is dyspnea. In some embodiments, the condition that can be relieved by causing vasodilation is scleroderma. In some embodiments, the condition is a risk of a cardiovascular condition, such as a risk of heart attack or stroke, or any of the conditions described above. In some embodiments, the condition is diabetic neuropathy. In some embodiments, the condition is pheochromocytoma or hyperadrenergic state. In some embodiments, a subject to which a CAII inhibitor or a CAII inhibitor and a composition comprising CAII (e.g., a composition comprising CAII and copper) is administered is a subject in need of vasodilation. In some embodiments, a subject in need of vasodilation is a subject undergoing radiation therapy. In some embodiments, a subject in need of vasodilation is a subject being treated with certain drugs, including but not limited to cancer therapeutics. In some embodiments, a subject in need of vasodilation is a subject undergoing surgery.

In some embodiments, “administering” or “administration” in the context of CAII inhibitors and CAII compositions (e.g., methods of administering CAII inhibitors and CAII compositions) means providing a material to a subject in a manner that is pharmacologically useful. In some embodiments, a CAII inhibitor or a CAII inhibitor and a composition comprising CAII (e.g., a composition comprising CAII and copper) is administered to a subject enterally. In some embodiments, an enteral administration of the composition is oral. In some embodiments, a CAII inhibitor or a CAII inhibitor and a composition comprising CAII (e.g., a composition comprising CAII and copper) is administered to the subject parenterally. In some embodiments, a CAII inhibitor or a CAII inhibitor and a composition comprising CAII (e.g., a composition comprising CAII and copper) is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a CAII inhibitor or a CAII inhibitor and a composition comprising CAII (e.g., a composition comprising CAII and copper) is administered to the subject by injection into the hepatic artery or portal vein. In embodiments in a CAII inhibitor and a composition comprising CAII (e.g., a composition comprising CAII and copper) are administered to a subject, the inhibitor and the composition comprising CAII may be administered via the same route or may be administered via different routes.

To “treat” a disease as the term is used herein in the context of CAII inhibitors and CAII compositions, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of a CAII inhibitor or a CAII inhibitor and a composition comprising CAII (e.g., a composition comprising CAII and copper) in this context may be an amount of the compound and/or composition that is capable of increasing nitrite reductase activity and/or increasing NO levels. A therapeutically acceptable amount may be an amount that is capable of treating a disease or condition, e.g., a disease or condition that that can be relieved by causing vasodilation, such as a condition described herein, including a heart condition (e.g., myocardial infarction, stroke), hypertension, pulmonary hypertension, erectile dysfunction, Raynaud's phenomenon, or muscular atrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a subject is administered a CAII inhibitor simultaneously with being administered a composition comprising CAII and copper. In some embodiments, A CAII inhibitor is administered immediately after (e.g., within 1 minute, within 2 minutes, within 5 minutes, within 15 minutes, within 20 minutes, within 25 minutes, within 30 minutes, within 1 h, within 2 h, within 3 h, within 4 h, within 5 h, within 6 h, within 7 h, within 8 h, within 9 h, within 10 h, within 11 h, within 12 h, within 18 h, or within 24 h) being administered the composition comprising CAII and copper. In some embodiments, A CAII inhibitor is administered immediately before (e.g., within 1 minute, within 2 minutes, within 5 minutes, within 15 minutes, within 20 minutes, within 25 minutes, within 30 minutes, within 1 h, within 2 h, within 3 h, within 4 h, within 5 h, within 6 h, within 7 h, within 8 h, within 9 h, within 10 h, within 11 h, within 12 h, within 18 h, or within 24 h) of being administered the composition comprising CAII and copper.

In some embodiments, a subject with a condition that can be relieved by causing vasodilation (e.g., a subject suffering from or at risk of suffering from a condition described herein, including but not limited to myocardial infarction, stroke, Raynaud's phenomenon, heart failure, angina, coronary artery disease, erectile dysfunction, hypertension, pulmonary hypertension or muscular atrophy) is prophylactically administered a CAII inhibitor so that when a composition comprising CAII and copper is administered to the subject, the composition comprising CAII and copper has nitrite reductase activity. Hypertension may be primary hypertension or secondary hypertension. Secondary hypertension may be secondary to eclampsia, preeclampsia, renovascular disease or renal disease, sleep apnea, or endocrine abnormalities.

In some embodiments, a carbonic anhydrase inhibitor is administered at a dose of 1 to 10000 mg daily (e.g., 1 to 9000 mg daily, 1 to 8000 mg daily, 1 to 7000 mg daily, 1 to 6000 mg daily, 1 to 5000 mg daily, 1 to 4000 mg daily, 1 to 3000 mg daily, 1 to 2000 mg daily, 1 to 1000 mg daily, 1 to 900 mg daily, 1 to 800 mg daily, 1 to 700 mg daily, 1 to 600 mg daily, 1 to 500 mg daily, 1 to 400 mg daily, 1 to 300 mg daily, 1 to 200 mg daily, 1 to 100 mg daily, 50 to 1000 mg daily, 100 to 1000 mg daily, 150 to 1000 mg daily, 200 to 1000 mg daily, 250 to 1000 mg daily, 300 to 1000 mg daily, 350 to 1000 mg daily, 400 to 1000 mg daily, 450 to 1000 mg daily, 500 to 1000 mg daily, 550 to 1000 mg daily, 600 to 1000 mg daily, 650 to 1000 mg daily, 700 to 1000 mg daily, 750 to 1000 mg daily, 800 to 1000 mg daily, 850 to 1000 mg daily, 900 to 1000 mg daily, 950 to 1000 mg daily, 100 to 950 mg daily, 100 to 900 mg daily, 100 to 850 mg daily, 100 to 800 mg daily, 100 to 750 mg daily, 100 to 700 mg daily, 100 to 650 mg daily, 100 to 600 mg daily, 100 to 550 mg daily, 100 to 500 mg daily, 100 to 450 mg daily, 100 to 400 mg daily, 100 to 350 mg daily, 100 to 300 mg daily, 100 to 250 mg daily, 100 to 200 mg daily, 100 to 150 mg daily, 150 to 950 mg daily, 150 to 900 mg daily, 150 to 850 mg daily, 150 to 800 mg daily, 150 to 750 mg daily, 150 to 700 mg daily, 150 to 650 mg daily, 150 to 600 mg daily, 150 to 550 mg daily, 150 to 500 mg daily, 150 to 450 mg daily, 150 to 400 mg daily, 150 to 350 mg daily, 150 to 300 mg daily, 150 to 250 mg daily, 150 to 200 mg daily, 200 to 950 mg daily, 200 to 900 mg daily, 200 to 850 mg daily, 200 to 800 mg daily, 200 to 750 mg daily, 200 to 700 mg daily, 200 to 650 mg daily, 200 to 600 mg daily, 200 to 550 mg daily, 200 to 500 mg daily, 200 to 450 mg daily, 200 to 400 mg daily, 200 to 350 mg daily, 200 to 300 mg daily, 200 to 250 mg daily, 250 to 950 mg daily, 250 to 900 mg daily, 250 to 850 mg daily, 250 to 800 mg daily, 250 to 750 mg daily, 250 to 700 mg daily, 250 to 650 mg daily, 250 to 600 mg daily, 250 to 550 mg daily, 250 to 500 mg daily, 250 to 450 mg daily, 250 to 400 mg daily, 250 to 350 mg daily, 250 to 300 mg daily, 300 to 950 mg daily, 300 to 900 mg daily, 300 to 850 mg daily, 300 to 800 mg daily, 300 to 750 mg daily, 300 to 700 mg daily, 300 to 650 mg daily, 300 to 600 mg daily, 300 to 550 mg daily, 300 to 500 mg daily, 300 to 450 mg daily, 300 to 400 mg daily, 300 to 350 mg daily, 350 to 950 mg daily, 350 to 900 mg daily, 350 to 850 mg daily, 350 to 800 mg daily, 350 to 750 mg daily, 350 to 700 mg daily, 350 to 650 mg daily, 350 to 600 mg daily, 350 to 550 mg daily, 350 to 500 mg daily, 350 to 450 mg daily, 350 to 400 mg daily, 400 to 950 mg daily, 400 to 900 mg daily, 400 to 850 mg daily, 400 to 800 mg daily, 400 to 750 mg daily, 400 to 700 mg daily, 400 to 650 mg daily, 400 to 600 mg daily, 400 to 550 mg daily, 400 to 500 mg daily, 400 to 450 mg daily, 450 to 950 mg daily, 450 to 900 mg daily, 450 to 850 mg daily, 450 to 800 mg daily, 450 to 750 mg daily, 450 to 700 mg daily, 450 to 650 mg daily, 450 to 600 mg daily, 450 to 550 mg daily, 450 to 500 mg daily, 500 to 950 mg daily, 500 to 900 mg daily, 500 to 850 mg daily, 500 to 800 mg daily, 500 to 750 mg daily, 500 to 700 mg daily, 500 to 650 mg daily, 500 to 600 mg daily, 500 to 550 mg daily, 550 to 950 mg daily, 550 to 900 mg daily, 550 to 850 mg daily, 550 to 800 mg daily, 550 to 750 mg daily, 550 to 700 mg daily, 550 to 650 mg daily, 550 to 600 mg daily, or any range or combination thereof). In some embodiments, a carbonic anhydrase inhibitor is administered at a dose of 250 mg to 1000 mg per day. In some embodiments, a carbonic anhydrase inhibitor is administered at a dose of 250 to 500 mg per day. In some embodiments, a carbonic anhydrase inhibitor is administered at a dose of 250 to 375 mg per day. In some embodiments, a carbonic anhydrase inhibitor is administered at a dose of up to 1000 mg per day. In some embodiments, a carbonic anhydrase inhibitor is administered at a dose of 120, 200, or 500 mg per administration (e.g., per day or per dose). In some embodiments, a carbonic anhydrase inhibitor is administered at a dose of 25 or 50 mg per administration (e.g., per day or per dose). In some embodiments, the dose is administered once daily, the dose is administered twice daily, or the dose is administered three times daily or more (e.g., the total daily dose is divided between two, three, or more administrations, or the individual dose is administered once, twice, three times or more daily).

### Methods of Administering an Inhibitor of CAII Esterase Activity

The inventors of the present disclosure have found that CAII has esterase activity by which CAII can degrade NSAIDs (e.g., aspirin) such as those administered to subjects with heart conditions (e.g., subjects having suffered, are suffering, or are at risk of suffering a myocardial infarction, stroke, Raynaud's phenomenon, or another condition described herein). For example, CAII converts aspirin to the acetylated form of aspirin. This results in a lower concentration of aspirin in the body that can perform its intended function (e.g., inhibition of COX). Therefore, by inhibiting the esterase activity of CAII, subjects who have been administered aspirin can have a higher amount of aspirin to perform the intended function (and thus a higher half-life of aspirin).

Accordingly, provided herein is a method comprising administering to a subject who is administered or is going to be administered a nonsteroidal anti-inflammatory drug (NSAID, e.g., aspirin or ibuprofen) an inhibitor of carbonic anhydrase II (CAII), wherein the CAII has esterase activity. There are numerous methods of measuring esterase activity (see e.g., Gilham et al. Methods. 2005 June; 36(2):139-47, Bardi iand Delfini., J. Inst. Brew., 1993, 99: 385, Peng et al. BioRes. 11(4), 10099-10111, www.sigmaaldrich.com/technical-documents/protocols/biology/enzymatic-assay-of-esterase.html, and academic.oup.com/clinchem/article-abstract/3/3/185/5664975?redirectedFrom=PDF)

Provided herein are methods comprising administering to a subject an inhibitor of CAII (e.g., CAII esterase activity) in an amount sufficient to reduce esterase activity of CAII.

In some embodiments, esterase activity of CAII is reduced by at least 10% (e.g., at least 10%, at least 20%, at least 30%, at least 40%, at least 50%, at least 60%, at least 70%, at least 80%, at least 90%, at least 95%, at least 98%, at least 99%, or at least 99.9%) either when tested in vitro or in vivo. In some embodiments, administering of a CAII inhibitor in a subject results in an increase in the half-life of an NSAID administered to the subject by at least 2% (e.g., at least 2%, at least 5%, at least 10%, at least 20%, at least 30%, at least 40%, at least 50%, at least 60%, at least 70%, at least 80%, at least 90%, at least 95%, at least 98%, at least 99%, at least 100% or more). In some embodiments, administering of a CAII inhibitor in a subject results in an increase in the half-life of an NSAID administered to the subject by at least two-fold (e.g., at least two-fold, at least three-fold, at least four-fold, at least five-fold, at least six-fold, at least seven-fold, at least eight-fold, at least nine-fold, at least 10-fold, at least 15-fold, at least 20-fold, or more).

In some embodiments, a subject is administered a CAII inhibitor to treat a heart condition (e.g., myocardial infarction, stroke, or Raynaud's phenomenon). As used herein, “treating” can include either therapeutic use or prophylactic use.

In some embodiments, an NSAID is a salicylate (e.g., aspirin, diflunisal, salsalate, or salicylic acid), a propionic acid derivative (e.g., Ibuprofen, Dexibuprofen, Naproxen, Ketoprofen, or Loxoprofen), an acetic acid derivative (e.g., Indomethacin, Tolmetin, or Etodolac), an enolic acid (oxicam) derivative (e.g., Piroxicam or Lornoxicam), an anthranilic acid derivative (a fenamate; e.g., Mefenamic acid or Flufenamic acid), a selective COX-2 inhibitor (a coxib; e.g., Celecoxib, Rofecoxib, or Valdecoxib), a sulfonanilides (e.g., Nimesulide). Some other non-limiting examples of NSAIDs include Clonixin, Licofelone, and H-harpagide.

In some embodiments, an inhibitor of CAII that is administered to a subject who is administered or is going to be administered a NSAID and/or is suffering from or is at risk of suffering from a heart condition is a compound that inhibitors esterase activity of CAII. In some embodiments, an inhibitor of CAII is a large biomolecule (e.g., a peptide, protein, or siRNA). In some embodiments, an inhibitor of CAII is a small molecule. Some non-limiting examples of CAII inhibitors include acetazolamide, methazolamide, ethoxzolamide, dichlorphenamide, dorzolamide, brinzolamide, topiramate, celecoxib, sulpiride, sulthiame, valdecoxib, zonisamide, irosustat, or esterone sulfamate, or a benzyl-sulfonamide compound.

In some embodiments, the subject who is administered or who is going to be administered an NSAID suffers from or is at risk of suffering from a condition that can be relieved by causing vasodilation. In some embodiments, a person at risk of suffering from a condition that can be relieved by causing vasodilation is a person who has been prescribed low-dose NSAID, a person determined by a medical doctor to be at high risk for conditions that can be relieved by causing vasodilation (e.g., on the basis of obesity, rate of smoking, heredity, or presence of genetic markers for such conditions). Non-limiting examples of conditions that can be relieved by vasodilation include hypertension, pulmonary hypertension, a heart condition (e.g., heart failure, angina, coronary artery disease, or myocardial infarction), erectile dysfunction, or muscular atrophy. Hypertension may be primary hypertension or secondary hypertension, wherein the secondary hypertension is secondary to eclampsia, preeclampsia, renovascular disease or renal disease, sleep apnea, or endocrine abnormalities. In some embodiments, the condition that can be relieved by causing vasodilation is a cardiovascular condition. In some embodiments, the cardiovascular condition is hypertension (e.g., high blood pressure), heart failure (e.g., acute heart failure, congestive heart failure, chronic heart failure), ischemic heart disease, pulmonary hypertension, pulmonary arterial hypertension (e.g., idiopathic pulmonary arterial hypertension or hereditary pulmonary arterial hypertension), chronic thromboembolic pulmonary hypertension, pulmonary edema, angina (e.g., angina pectoris), unstable angina, chronic stable angina, coronary artery disease, myocardial infarction (e.g., acute myocardial infarction), cardiomyopathy, erectile dysfunction, muscle atrophy, preeclampsia or eclampsia. In some embodiments, the condition that can be relieved by causing vasodilation is an acute coronary syndrome. In some embodiments, the condition that can be relieved by causing vasodilation is myocardial infarction. In some embodiments, the condition that can be relieved by causing vasodilation is hypertension. In some embodiments, the condition that can be relieved by causing vasodilation is peripheral arterial disease or peripheral vascular disease. In some embodiments, the condition is Raynaud's disease or Raynaud's phenomenon. In some embodiments, the condition that can be relieved by causing vasodilation is dyspnea. In some embodiments, the condition that can be relieved by causing vasodilation is scleroderma. In some embodiments, the condition is a risk of a cardiovascular condition, such as a risk of heart attack or stroke, or any of the conditions described above. In some embodiments, the condition is diabetic neuropathy. In some embodiments, the condition is pheochromocytoma or hyperadrenergic state. In some embodiments, a subject to which a CAII inhibitor is administered is a subject in need of vasodilation. In some embodiments, a subject in need of vasodilation is a subject undergoing radiation therapy. In some embodiments, a subject in need of vasodilation is a subject being treated with certain drugs, including but not limited to cancer therapeutics. In some embodiments, a subject in need of vasodilation is a subject undergoing surgery.

In some embodiments of any one of the methods of administering a CAII inhibitor to a subject as provided herein comprises administering a CAII inhibitor to a subject having or at risk of having a heart condition. Non-limiting examples of a heart condition of a subject who is administered a CAII inhibitor according to any one of the methods of administering as disclosed herein are myocardial infarction, stroke, and Raynaud's phenomenon. In some embodiments, a subject to which a CAII inhibitor is administered, has been administered or is going to be administered a NSAID.

In some embodiments, a subject is administered a CAII inhibitor simultaneously with being administered the NSAID. In some embodiments, A CAII inhibitor is administered immediately after (e.g., within 1 minute, within 2 minutes, within 5 minutes, within 15 minutes, within 20 minutes, within 25 minutes, within 30 minutes, within 1 h, within 2 h, within 3 h, within 4 h, within 5 h, within 6 h, within 7 h, within 8 h, within 9 h, within 10 h, within 11 h, within 12 h, within 18 h, or within 24 h) being administered the NSAID (e.g., aspirin). In some embodiments, A CAII inhibitor is administered immediately before (e.g., within 1 minute, within 2 minutes, within 5 minutes, within 15 minutes, within 20 minutes, within 25 minutes, within 30 minutes, within 1 h, within 2 h, within 3 h, within 4 h, within 5 h, within 6 h, within 7 h, within 8 h, within 9 h, within 10 h, within 11 h, within 12 h, within 18 h, or within 24 h) of being administered the NSAID (e.g., aspirin).

In some embodiments, a subject with a heart condition (e.g., suffering from or at risk of suffering from myocardial infarction, stroke, or Raynaud's phenomenon) is prophylactically administered a CAII inhibitor so that when a NSAID is administered to the subject, the NSAID is not degraded by CAII.

In some embodiments, an NSAID is administered at a dose of 50 to 1000 mg per dose (e.g., 100 to 1000 mg per dose, 150 to 1000 mg per dose, 200 to 1000 mg per dose, 250 to 1000 mg per dose, 300 to 1000 mg per dose, 350 to 1000 mg per dose, 400 to 1000 mg per dose, 450 to 1000 mg per dose, 500 to 1000 mg per dose, 550 to 1000 mg per dose, 600 to 1000 mg per dose, 650 to 1000 mg per dose, 700 to 1000 mg per dose, 750 to 1000 mg per dose, 800 to 1000 mg per dose, 850 to 1000 mg per dose, 900 to 1000 mg per dose, 950 to 1000 mg per dose, 100 to 950 mg per dose, 100 to 900 mg per dose, 100 to 850 mg per dose, 100 to 800 mg per dose, 100 to 750 mg per dose, 100 to 700 mg per dose, 100 to 650 mg per dose, 100 to 600 mg per dose, 100 to 550 mg per dose, 100 to 500 mg per dose, 100 to 450 mg per dose, 100 to 400 mg per dose, 100 to 350 mg per dose, 100 to 300 mg per dose, 100 to 250 mg per dose, 100 to 200 mg per dose, 100 to 150 mg per dose, 150 to 950 mg per dose, 150 to 900 mg per dose, 150 to 850 mg per dose, 150 to 800 mg per dose, 150 to 750 mg per dose, 150 to 700 mg per dose, 150 to 650 mg per dose, 150 to 600 mg per dose, 150 to 550 mg per dose, 150 to 500 mg per dose, 150 to 450 mg per dose, 150 to 400 mg per dose, 150 to 350 mg per dose, 150 to 300 mg per dose, 150 to 250 mg per dose, 150 to 200 mg per dose, 200 to 950 mg per dose, 200 to 900 mg per dose, 200 to 850 mg per dose, 200 to 800 mg per dose, 200 to 750 mg per dose, 200 to 700 mg per dose, 200 to 650 mg per dose, 200 to 600 mg per dose, 200 to 550 mg per dose, 200 to 500 mg per dose, 200 to 450 mg per dose, 200 to 400 mg per dose, 200 to 350 mg per dose, 200 to 300 mg per dose, 200 to 250 mg per dose, 250 to 950 mg per dose, 250 to 900 mg per dose, 250 to 850 mg per dose, 250 to 800 mg per dose, 250 to 750 mg per dose, 250 to 700 mg per dose, 250 to 650 mg per dose, 250 to 600 mg per dose, 250 to 550 mg per dose, 250 to 500 mg per dose, 250 to 450 mg per dose, 250 to 400 mg per dose, 250 to 350 mg per dose, 250 to 300 mg per dose, 300 to 950 mg per dose, 300 to 900 mg per dose, 300 to 850 mg per dose, 300 to 800 mg per dose, 300 to 750 mg per dose, 300 to 700 mg per dose, 300 to 650 mg per dose, 300 to 600 mg per dose, 300 to 550 mg per dose, 300 to 500 mg per dose, 300 to 450 mg per dose, 300 to 400 mg per dose, 300 to 350 mg per dose, 350 to 950 mg per dose, 350 to 900 mg per dose, 350 to 850 mg per dose, 350 to 800 mg per dose, 350 to 750 mg per dose, 350 to 700 mg per dose, 350 to 650 mg per dose, 350 to 600 mg per dose, 350 to 550 mg per dose, 350 to 500 mg per dose, 350 to 450 mg per dose, 350 to 400 mg per dose, 400 to 950 mg per dose, 400 to 900 mg per dose, 400 to 850 mg per dose, 400 to 800 mg per dose, 400 to 750 mg per dose, 400 to 700 mg per dose, 400 to 650 mg per dose, 400 to 600 mg per dose, 400 to 550 mg per dose, 400 to 500 mg per dose, 400 to 450 mg per dose, 450 to 950 mg per dose, 450 to 900 mg per dose, 450 to 850 mg per dose, 450 to 800 mg per dose, 450 to 750 mg per dose, 450 to 700 mg per dose, 450 to 650 mg per dose, 450 to 600 mg per dose, 450 to 550 mg per dose, 450 to 500 mg per dose, 500 to 950 mg per dose, 500 to 900 mg per dose, 500 to 850 mg per dose, 500 to 800 mg per dose, 500 to 750 mg per dose, 500 to 700 mg per dose, 500 to 650 mg per dose, 500 to 600 mg per dose, 500 to 550 mg per dose, 550 to 950 mg per dose, 550 to 900 mg per dose, 550 to 850 mg per dose, 550 to 800 mg per dose, 550 to 750 mg per dose, 550 to 700 mg per dose, 550 to 650 mg per dose, 550 to 600 mg per dose, or any range or combination thereof). In some embodiments, an NSAID is administered at a dose of 325 mg. In some embodiments, an NSAID is administered at a dose of 250 to 500 mg. In some embodiments, an NSAID is administered at a dose of 200 to 800 mg. In some embodiments, an NSAID is administered at a dose of up to 3200 mg per day. In some embodiments, the dose is administered once daily, the dose is administered twice daily, or the dose is administered three times daily or more (e.g., the total daily dose is divided between two, three, or more administrations, or the individual dose is administered once, twice, three times or more daily).

In some embodiments, “administering” or “administration” in the context of CAII inhibitors means providing a material (e.g., a CAII inhibitor) to a subject in a manner that is pharmacologically useful. In some embodiments, a CAII inhibitor is administered to a subject enterally. In some embodiments, an enteral administration of the essential metal element/s is oral. In some embodiments, a CAII inhibitor is administered to the subject parenterally. In some embodiments, a CAII inhibitor is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a CAII inhibitor is administered to the subject by injection into the hepatic artery or portal vein.

To “treat” a disease as the term is used herein in the context of CAII inhibitors, means to reduce the frequency or severity of at least one sign or symptom of a disease or disorder experienced by a subject. The compositions described above (e.g., CAII inhibitors) or elsewhere herein are typically administered to a subject in an effective amount, that is, an amount capable of producing a desirable result. The desirable result will depend upon the active agent being administered. For example, an effective amount of CAII inhibitor may be an amount of the CAII inhibitor that is capable of activating or inhibiting an amount of CAII enzymatic activity (e.g., esterase activity, nitrite reductase activity or carbonic anhydrase activity), or an amount of CAII inhibitor that is capable of inhibiting or decreasing the rate of hydrolysis of aspirin facilitated by CAII. A therapeutically acceptable amount may be an amount that is capable of treating a disease or condition that can be relieved by causing vasodilation, such as a condition described herein, including a heart condition (e.g., myocardial infarction or stroke), hypertension, pulmonary hypertension, erectile dysfunction, Raynaud's phenomenon, or muscular atrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

As used herein, a subject who “is going to be administered” a therapeutic (e.g., an NSAID, a carbonic anhydrase inhibitor, and/or a CAII composition) is a subject who has been prescribed the therapeutic, a subject who is at elevated risk for a condition for which the therapeutic may be prescribed relative to the general population, or a subject who has been diagnosed with a condition for which the therapeutic may be prescribed. In some embodiments, a subject who is going to be administered a therapeutic is a subject who is at risk of a cardiovascular disease. In some embodiments, a subject who is going to be administered a therapeutic is a subject who has been diagnosed with a cardiovascular disease. For example, a subject who is going to be administered an NSAID is in some embodiments a subject who has been prescribed an NSAID, a subject who is at elevated risk of a cardiovascular disease relative to the general population, or a subject who has been diagnosed with a cardiovascular disease.

### EXAMPLES

### Example 1: Structure and Mechanism of Copper-Carbonic Anhydrase II: A Nitrite Reductase

This example discusses the nitrite reductase activity of CAII.

Nitric oxide (NO) promotes vasodilation through the activation of guanylate cyclase, resulting in the relaxation of vasculature smooth muscle and subsequent decrease in blood pressure. Therefore, its regulation is of interest for the treatment and prevention of heart disease. An example is pulmonary hypertension which is treated by targeting this NO/vasodilation pathway. In bacteria, plants, and fungi, nitrite (NO2-) is utilized as a source of NO through enzymes known as nitrite reductases. These enzymes reduce NO2- to NO through a catalytic metal ion, often copper. Recently, several studies have shown nitrite reductase activity of mammalian carbonic anhydrase II (CAII), yet the molecular basis for this activity is unknown. Here the crystal structure of copper bound human CAII (Cu-CAII) in complex with NO2- at 1.2 Å resolution is reported. The structure exhibits Type 1 (T-1) and 2 (T-2) copper centers, analogous to bacterial nitrite reductases, both required for catalysis. The copper-substituted CAII active site is penta-coordinated with a “side-on” bound NO2-, resembling a T-2 center. At the N-terminus, several residues that are normally disordered form a porphyrin ring-like configuration surrounding a second copper, acting as a T-1 center. A structural comparison to both apo- (without metal) and Zn-CAII, provides a mechanistic picture of how, in the presence of copper, CAII, with minimal conformational changes, can function as a nitrite reductase. In mammals (including humans), it has been well established Zn-Carbonic anhydrase (Zn-CA) catalyzes the reversible hydration/dehydration of carbon dioxide (CO₂)/bicarbonate (HCO₃⁻) (1, 2). There are 12 enzymatically active CA isoforms in humans, with CAI and II abundant in most cells, especial in red blood cells (RBC), and as such directly involved in gas exchange, ion transport, and extra- and intracellular pH regulation (3). A single Zn-CAI and II protein is capable of converting ˜0.2 and 1.1×10⁶ CO₂ to HCO₃⁻ per second, respectively (4, 5). Hence, with a concentration of Zn-CAI and II of 4.2×10⁶ and 4.8×10⁵ molecules per RBC, there are excessive amounts of CAs to regulate the 5×10²⁰ CO₂ generated in an adult human breath (6). This excess of CA in the blood lends to the question do carbonic anhydrases have other regulatory roles? Many reports have shown CAII is a promiscuous enzyme, capable of binding multiple substrates and performing a variety of reactions besides its carbonic anhydrase activity. These include: binding other gaseous molecules such as nitrate, nitrite and molecular oxygen, esterase activity with many ester containing compounds, and hydration reactions such as hydrating cyanamide to urea (7— 10). While these activities are important and show CAII's robust role, recent reports, including (11-12), are inconclusive as whether CAII can produce Nitric oxide (NO) through Nitrite (NO₂⁻) reduction, and thus, regarding its role in vasodilation and regulation of blood pressure.

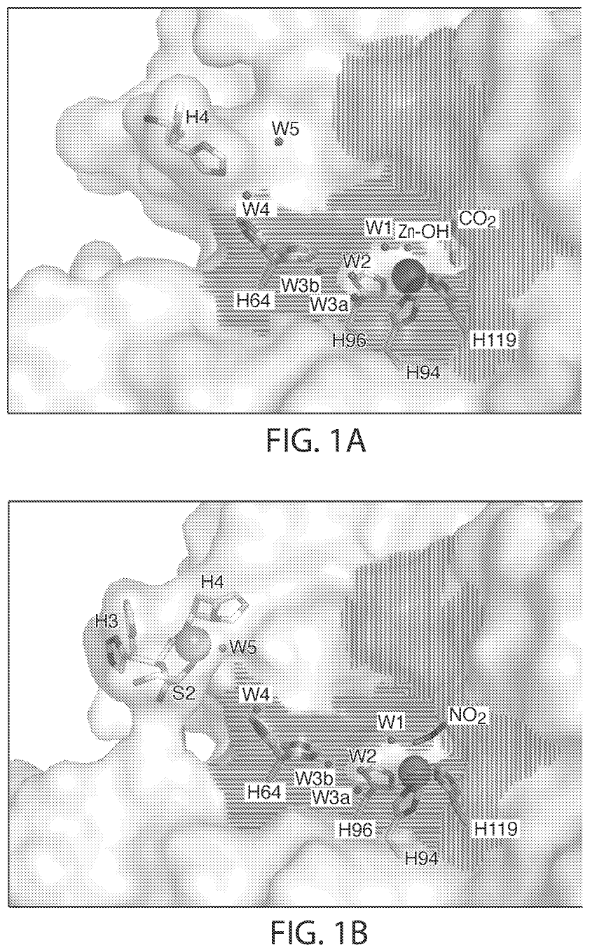

CAII is a 30 kDa protein, with a solvent exposed active site. In Zn-CAII, the zinc is tetrahedrally coordinated by three histidines (H94, H96, and H119) and a solvent molecule (13). The active site is divided into a distinct hydrophobic and hydrophilic side. The hydrophobic side (residues I91, V121, F131, V135, L141, V143, L198, P202, L204, V207, and W209) stabilizes the CO₂ substrate, while the hydrophilic side (N62, H64, N67, Q92, T199, and T200) orders and regulates the solvent (W1, W2, W3A, W3B, and WD (deep water)) required for rapid catalytic turnover(3). Of special importance is H64, that modulates between an “in” and “out” conformation (referring to its direction relative to the active site), and is known to be important in proton transfer (FIG. 1A) (14). Of note, all deposited structures of Zn-CAII to date have disordered N-termini (residues 1-4). The role of Zn-CAII in the hydration/dehydration of CO₂/HCO₃⁻ has been extensively studied. The reaction is a two-step, ping-pong mechanism. In the hydration direction, the first step is the nucleophilic attack of CO₂ by a Zn-bound hydroxyl that results in the formation of HCO₃⁻, which is displaced by a water molecule (15). The second step of the reaction, is the transfer of a proton from the Zn-bound water to the bulk solvent via the well-defined solvent network and H64 (16). The regeneration of the Zn-bound hydroxyl permits the catalytic reaction cycle, the k_{cat}/Kₘ of the reaction is 120 M⁻¹ μs⁻¹, which means Zn-CAII has evolved to near catalytic perfection for the hydration/dehydration of CO₂/HCO₃⁻, as it is diffusion rate limited (17).

In humans, the most common source of NO is its synthesis by endothelial nitrogen oxide synthase (eNOS), which catalyzes the oxidation of arginine to produce NO and citrulline (18). While this enzyme is responsible for NO production under normoxia, under hypoxic conditions the enzyme is acatalytic (19). Thus, other less understood pathway(s) have been suggested to function in the place of eNOS during times of low oxygen to produce NO through a nitrite reduction pathway. Nitrite represents an “untapped” source of NO in the blood with little understanding of how it is reduced. Although, previous studies have suggested hemoglobin or a CA as likely candidates as the nitrite reductase (19, 20).

Bacterial copper nitrite reductases utilize two separate and distinct copper binding sites to catalyze the reduction of nitrite. The first copper site, known as the Type I (T-1) site and coordinated by a cysteine, a methionine, and two histidines, functions to transfer electrons to the second copper site termed the Type II (T-2) site (FIG. 26) (21). The T-2 site, coordinated by three histidines and a solvent molecule, is where the nitrite reduction reaction occurs (21). It is interesting to note, previous studies have commented on the striking similarity between the Zn-CAII active site and bacterial nitrite reductase T2 sites, suggesting that CAII may be involved in mammalian nitrite reduction (22). In addition, recent studies have shown that bovine CAII can reduce NO₂⁻ to NO (12). However, when dialyzed with EDTA, the enzyme retained its carbonic anhydrase activity, yet lost its nitrite reductase activity (11). While zinc is a divalent cation, it has a full d orbital when coordinated in CAII, and thus, is unable to perform redox reactions. The two observations, taken together, suggest that there may be a factor in blood that activates the nitrite reductase activity of CAII. In blood, there is a relatively high concentration of copper (˜15 uM), which can replace the zinc in the CAII active site, as previous research has shown that CAII preferentially binds copper with 50-fold specificity over zinc (23-25). Hence, based on the knowledge of copper-containing bacterial nitrite reductases, it was hypothesized that copper was the additional cofactor in blood responsible for the nitrite reductase activity of CAII previously reported. Therefore, the addition of copper to apo-CAII (without metal) could be the mechanism to convert CAII to a nitrite reductase. In this study, the crystal structures of Cu— were compared to both apo- and Zn-CAII (see methods), to obtain a mechanistic picture of how in the presence of copper CAII can, with minimal conformational changes, be converted to a nitrite reductase.

### Results

Mammalian CAs selectively use zinc as their catalytic metal ion, using it as a Lewis acid to increase the nucleophilic character of the zinc bound hydroxyl. The CA active site, as described above, has the same characteristics as a Type II copper binding site in bacterial nitrite reductases: three coordinating histidines, polar residues for charged transition state stabilization, and metal bound solvent molecules. The crystal structure solved here confirmed this, with the Cu-CAII T-2 site having the same general conformation as the zinc active site. The copper atom is penta-coordinated via the three histidine residues (H94, H96, and H119), and a nitrite molecule, bound in a “side-on” conformation, coordinated via an oxygen and nitrogen (FIGS. 1A-1B). The copper-substituted active site forms a T-2 site perfectly as described in bacterial copper nitrite reductases.

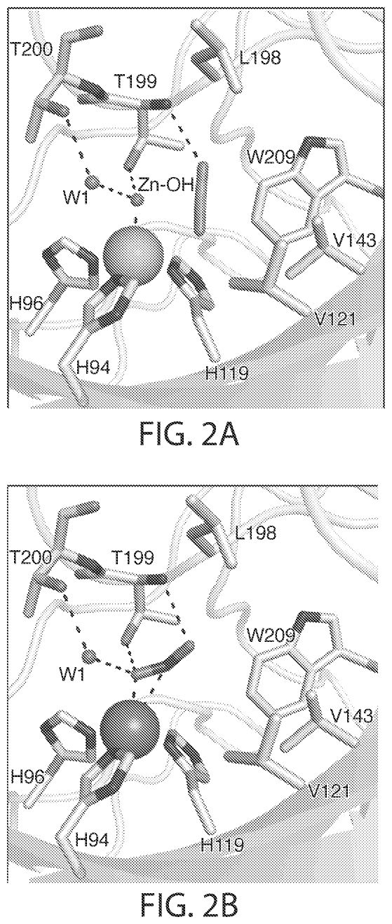

An ordered water network exists within Zn-CAII, responsible for rapid proton transfer (FIG. 1A). In the active site, the Cu-CAII has a slightly altered water network compared to the Zn-CAII structure (FIGS. 1A-1B), in that the zinc-bound solvent is replaced with a bound NO₂⁻ molecule, in a “side-on” configuration (FIGS. 2A-2B). This was unexpected, as no nitrite or nitrogen source was added to the crystallization conditions (1.6M sodium citrate and 50 mM Tris at a pH of 7.8). However, previous structural studies of bacterial copper nitrite reductases have revealed endogenous ligands bound to the T2 site (26, 27). Both NO₂⁻ and NO have been shown to be bound in Achromobacter cycloclastes T2 copper site, without being added to the crystal (FIG. 26). While the origin of these ligands is unknown, others have hypothesized synchrotron radiation as a possible source of high energy ions leading to the formation of these molecules (27). One of the NO₂⁻ oxygens occupies the position of the Zn-CAII deep water, important for solvent replenishment (28, 29). Comparison of the Zn-CAII:CO₂ (PDB:5YUI) to the Cu-CAII: NO₂⁻ complex, shows significant differences. While the NO₂⁻ binds directly to the copper and forms stabilizing interactions with the hydrophilic pocket, the CO₂ binds in a “side-on” conformation adjacent to the zinc and is stabilized by the hydrophobic pocket (FIG. 2). The NO is stabilized via hydrogen-bonding to residues T199 and T200 while also interacting with W1 (FIG. 2B). The CO₂ binding shares the same hydrogen bond to T199 while also forming hydrophobic interactions with V121, V143, and W209 (FIG. 2A). The Cu-CAII active site retains the same W1, W2, W3a, and W3b positions as Zn-CAII (FIGS. 1A-1B). However, in Cu-CAII an extended ordered water network exists spanning past H64, forming a hydrogen-bonding network up to the second copper binding site located at the N-terminus. This network is achieved with the ordering of two additional waters compared to the Zn-CAII, named W4 and W5 (FIG. 1B). Presumably, the additional water molecules complete a solvent network to span the two copper binding sites, allowing the electron transfer necessary for the nitrite reductase reaction (FIG. 1B).

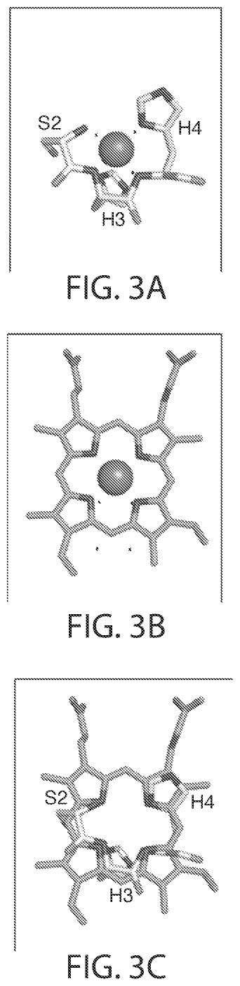

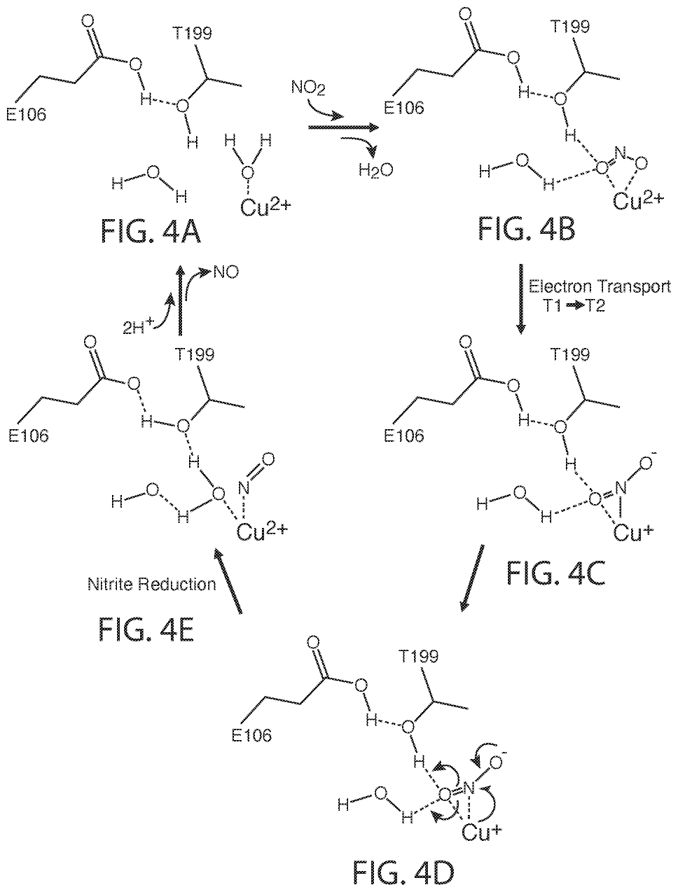

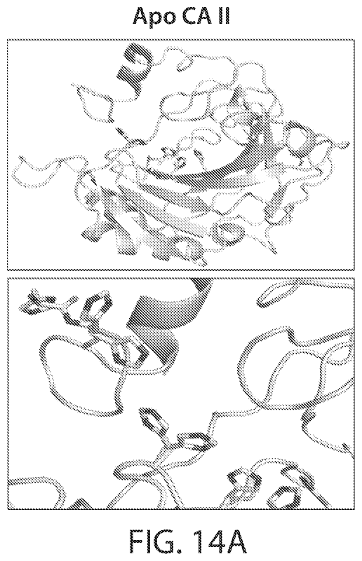

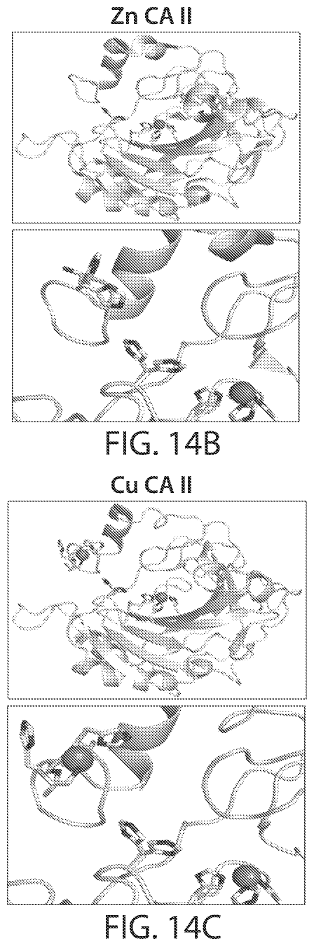

Mammalian CAIIs have a unique conserved N-terminus sequence (MSHHW), not observed in the other CA isoforms (uniprot.org). However, as previously mentioned, this sequence is disordered in all the Zn-CAII crystal structures deposited in the protein databank (FIG. 3A).(30, 31) The high-resolution structure of Zn-CAII (PDB:3KS3) only shows order of the N-terminus starting at H4, while the apo-CAII structures show some order also of H3 (FIG. 14). However, in the Cu-CAII structure, the N-terminus becomes ordered, forming a pseudo porphyrin ring, with the copper coordinated by several nitrogens (FIG. 3A). Previous work using paramagnetic NMR techniques and X-ray absorption spectroscopy predicted this N—Terminal structure as an Amino Terminal Copper and Nickel (ATCUN) binding motif as a high affinity binding site for copper, K_{d}˜0.5 nM.(32) As confirmed from the X-ray crystallography, this structure acts as the T-1 copper site, coordinated to the main chain nitrogens of S2, H3, and H4 (FIG. 3A). It is more than likely this site serves as the site of electron transfer to the T-2 copper active site (FIG. 1B). The pseudo porphyrin ring conformation, formed by the Cu-CAII N-terminus has a striking resemblance to that of heme-containing nitrite reductases (FIG. 3B). Structural superposition of the Cu-CAII N-terminus with Pseudomonas aeruginosa nitrite reductase heme, gave a RMSD of 0.3 Å (FIG. 3C). While not a porphyrin ring structure, this pseudo T-1 site, which has not previously been observed in metal-CAII structures, would provide the necessary electron donor site required for nitrite reduction. Hence, this structure provides an obvious mechanism for Cu-CAII to function as a nitrite reductase. Based on published mechanistic studies with bacterial nitrite reductase, the Cu-CAII active site has the T-1 and T-2 sites, the necessary bridging waters, and acidic residues to stabilize NO₂⁻ and its intermediates, thus catalyzing nitrite reduction (FIGS. 4A-4E). (18, 21)

As previously reported through NMR and X-ray Crystallography studies, copper substituted CAII has two binding sites for the metal cation, one in the canonical active site and one near the N-terminus (24, 32). However, previous reports showed the secondary copper binding site to be between His4 and His64, while the present data, for the first time, shows a conformational change in the N-terminus, forming a pseudo-porphyrin ring to bind a second copper. This new site is of mechanistic importance, as the proton shuttling residue His64, is free to undergo its conformational change, required for carbonic anhydrase activity. Zn-CAII is known to be inhibited by copper, which coincides with the His4-His64 binding site reported by the PDB entry 5EO1 (33). The presently disclosed work showed if zinc is bound in the active site, copper binds through His64, thus proton transfer is inhibited. Interestingly, the present disclosure shows if both sites are occupied by copper, His64 is unperturbed, allowing proton- or hypothetically electron-transfer between the two copper sites.

The water network within CAII has been extensively studied through structural and kinetic experiments (34-36). Previously to this work, the ordered water network was thought to have stopped at the proton shuttling residue His64. However, if copper is bound in both binding sites, it was shown that ordered water network further extends past His64 connecting the N-terminal binding site to the canonical binding site through a series of hydrogen bonds. Bacterial nitrite reductases are also known to also have ordered waters within their active sites responsible for proton donation/acceptance and ordering substrate binding, similarly to the water network of CAII (21). Marcus theory was originally developed to study the rate of electron transfer between ions (37). The theory is used to determine activation energy in simple systems by calculating reorganization energies upon electron transfer. This takes into account the donor and acceptors size and distance apart, as well as dielectric constants and charge transferred (37). This powerful tool can be used to study the dynamics and movement of electrons in solution. While originally only used for simple solutions, in the 90 s it was applied to large scale biological systems such as proteins, to calculate energy barriers in enzymatic function involved with electron transfer. In 1993 however, Dr. Silverman applied Marcus theory to Carbonic Anhydrase to calculate the activation energy associated with proton transfer (38). As previously mentioned, CA has an ordered water network with spanning hydrogen bonds that connect the zinc bound hydroxyl to bulk solvent. With some modifications, Dr. Silverman applied the Marcus theory to this water network to calculate the activation energy associated with the proton transfer step of the CA mechanism (38). He showed that this modified Marcus theory very accurately predicted the observed rates of CA proton transfer. However, Marcus theory has seen little success accurately predicting proton transfer except in one other enzyme, cytochrome c oxidase (39). This protein, like CA, is involved with both proton and electron transfer. Perhaps one reason Marcus theory so accurately predicts CA proton transfer, is that the water network is optimized for both proton and electron transfer depending on the metal cation bound, thus making the theory interchangeably work with either electrons or protons.

While it is accepted that zinc bound CAII exists in the blood, there is currently no direct experimental evidence to suggest the existence of copper bound CAII. However, previous work from Aamand et al. showed that bovine CAII, when purified from bovine blood, has nitrite reductase activity (12). Furthermore, if this bovine CAII was dialyzed against EDTA, the nitrite reductase activity was ablated indicating a metal cofactor within the bovine blood was needed for the CAII dependent nitrite reductase activity (11). The work presented here indicates the metal cofactor is copper, thus strongly supporting the existence of Cu-CAII in blood. Furthermore, with the known concentration of copper in blood, paired with the high affinity copper binding sites, it is extremely likely that many CAII molecules exist with two bound copper atoms. Zinc is one of the most abundant trace metals in the blood, with typical concentrations of ˜96 μM. However, copper also has relatively high concentrations in blood, typically ˜15 μM, 7-fold lower than zinc. (23) Previous work using a colorimetric 4-(2-pyridylazo)resorcinol assay showed the CAII K_{d} for copper is 17 fM, while zinc K_{d} is 800 fM, indicating that CAII has 50-fold specificity toward copper over zinc (25). Furthermore, this research from Hunt et al. indicated that while the affinity is higher for copper than zinc, copper removal from the active site is facilitated through EDTA while EDTA has no effect of zinc removal from the active site (25). This coincides with the previous observation that EDTA prevented nitrite reductase activity (copper dependent) while not affecting carbonic anhydrase activity (zinc dependent) (11). The high CAII affinity for copper over zinc is a well-documented phenomenon, with many papers proving that CAII's affinity is much higher for copper than zinc. (25, 40-42)

$${X}_{i} = \frac{[ {M}_{i} ] {K}_{i}}{1 + \sum [ {M}_{i} ] {K}_{i}}$$

Using the calculations outlined by Thompson et al. and the affinities and concentrations from above, it was predicted that ˜86% of CAII in the blood will have copper bound in the canonical active site (40). With the secondary site having an approximate K_{d} of 500 nM to copper, it was suspected that there was a substantial amount of bound Cu-CAII in the blood (32).

This study provides a feasible mechanistic view of how Cu-CAII can function as a nitrite reductase, given the physiological concentrations of CAII and copper in the blood. CAII has the conformational ability to switch activity from a carbonic anhydrase to a nitrite reductase, dependent, on the metal ion availability, Formation of Cu-CAII may explain nitrite reduction by cells under hypoxic conditions, allowing the formation of NO in RBCS.

### Materials & Methods

Human CAII was expressed and purified according to previously published protocols (43, 44). Briefly, a CAII gene-containing plasmid under control of a T7 promoter was transformed into competent BL21 E. coli cells via a standard BL21 transformation protocol. Following transformation, the E. coli cells were grown overnight in 100 mL of nutrient-rich Luria Broth. Cells were then transferred to a large-scale 1 L culture in the presence of selecting antibiotic and allowed to grow to an optical density of 0.6 at 600 nm. The cells were then induced by the addition of 0.5 mM isopropyl β-D-1-thiogalactoside (IPTG) and 1 mM zinc sulfate and incubated for an additional 3 hours. The zinc was added to aid in protein expression and folding, thus improving the yield. The cells were pelleted via centrifugation and subsequently lysed using a microfluidizer set to 18,000 PSI. The Zn-CAII was purified from the cell lysate using affinity chromatography with a p-aminomethyl-benzenesulfonamide affinity column. The final protein stock was buffer exchanged with storage buffer (50 mM Tris; pH 7.8) using a centrifugal filter. Purity was determined with SDS-PAGE and protein concentration was determined by UV/Vis spectroscopy at 280 nm.

In order to generate the copper substituted CAII, the first step was to remove the zinc, generating apo-CAII. Immediately following purification, Zn-CAII was diluted to a concentration of 1 mg/mL in storage buffer and incubated with 5× chelation buffer (500 mM pyridine-2,6-dicarboxylic acid; 125 mM MOPS; pH 7.0). The solution then gently stirred overnight at room temperature (20° C.) and then passed over the p-aminomethyl-benzenesulfonamide affinity column. Any residual Zn-CAII attached to the column, while the apo-CAII was collected in the flow through. The apo-CAII was buffer exchanged using centrifugal filters against storage buffer to remove any residual chelating agent. The loss of enzyme activity was verified using a standard colorimetric esterase based kinetic method, as described elsewhere (45).

Apo-CAII crystals were grown via the hanging drop vapor-diffusion method. Crystal trays were set up with 500 uL of mother liquor in the well, containing 1.6M sodium citrate and 50 mM Tris at a pH of 7.8. Hanging drops of 9 μL were utilized consisting of a 1:1 ratio of 10 mg/mL protein to mother liquor. Crystal trays were left undisturbed at room temperature (RT) and apo-CAII crystal growth was observed after three days. To generate copper-substituted CAII crystals, the preformed apo-CAII crystals were incubated with 1 μL of a 10 mM stock solution of CuCl₂ in the hanging drops. The addition of 10 mM CuCl₂ did not result in osmotic shock to the crystals, however concentrations greater than 50 mM resulted in cracked, brittle crystals.

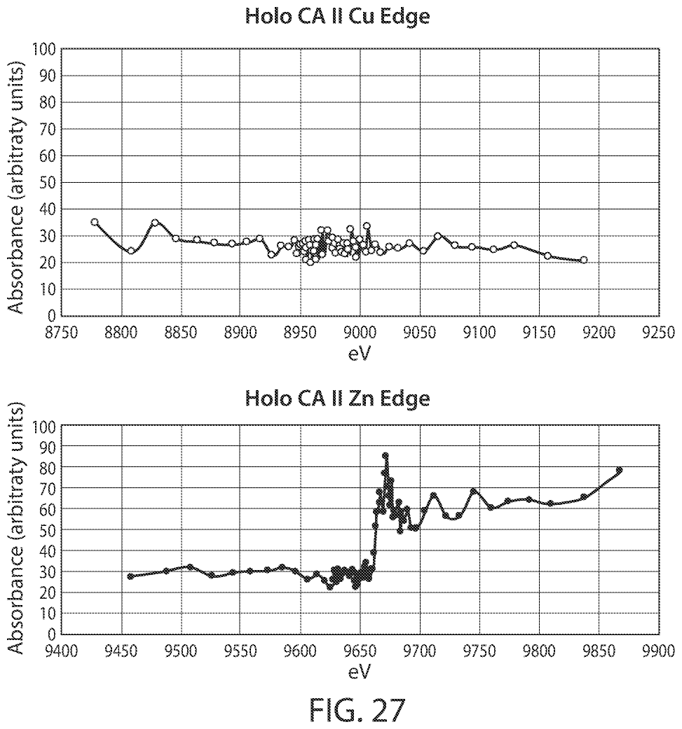

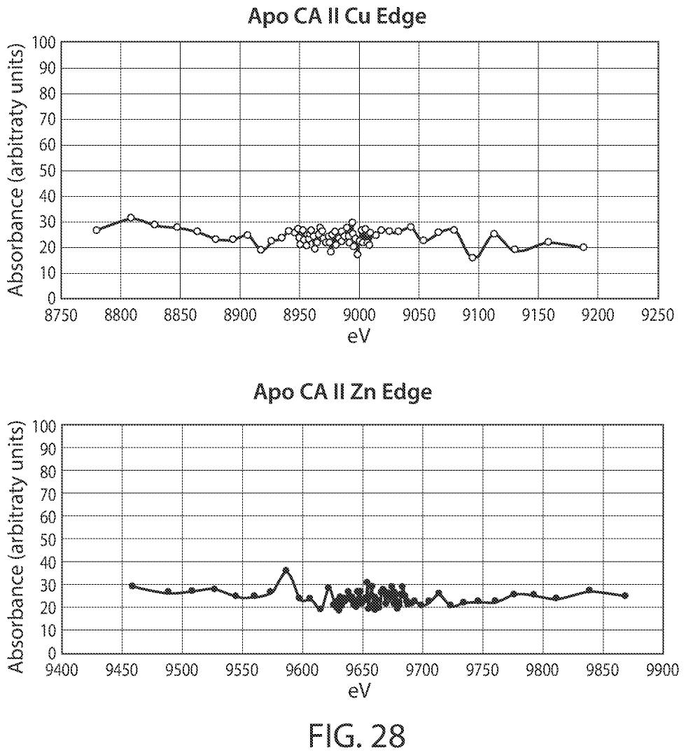

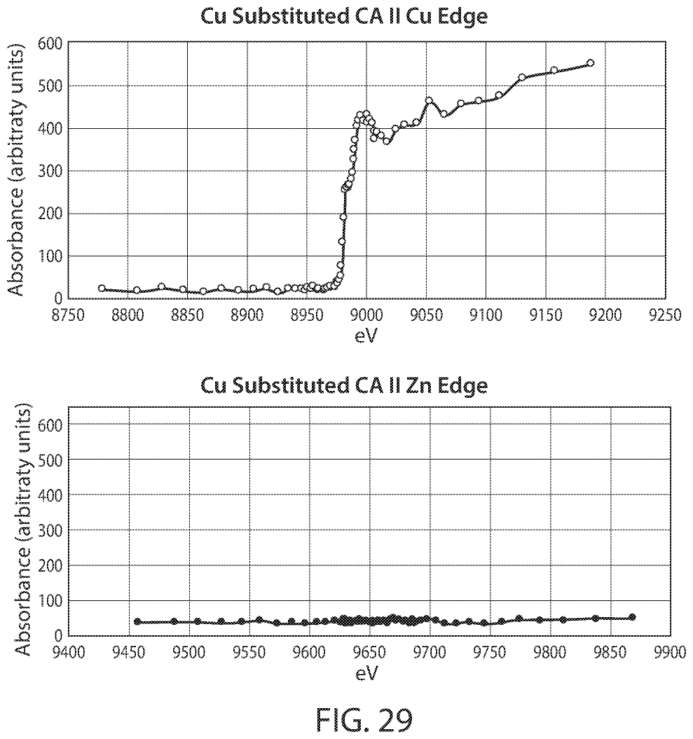

The Zn-CAII crystals were grown in the same fashion as the apo-CAII crystals. Crystal trays were set up with 500 uL of mother liquor in the well, containing 1.6M sodium citrate and 50 mM Tris at a pH of 7.8. 5 μL hanging drops were utilized consisting of a 1:1 ratio of 10 mg/mL protein to mother liquor. Crystal trays were left undisturbed at RT and Zn-CAII crystal growth was observed the next day. The crystals were harvested utilizing Mitegen loops, flash cooled in liquid nitrogen, and shipped to Stanford Synchrotron Radiation Lightsource (SSRL). Data was collected at the 9-2 beamline at SSRL, using a Pilatus 6M detector with 0.3° oscillations, a wavelength of 0.9795 Å, and detector distance depending on the resolution of the crystal diffraction. Each data set consisted of 600 images for a total of 180° data. X-ray absorption spectroscopy was also performed at the 9-2 beamline to determine the presence of copper or zinc in the respective crystals (FIGS. 27, 28 and 29).

The diffraction images were indexed and integrated using XDS, then merged and scaled to the P2₁ space group, using the program Aimless via the CCP4 program suite (46, 47). The diffraction data was phased with standard molecular replacement methods using the software package PHENIX using the CAII PDB entry 3KS3 as the search model (48). Coordinate refinements were calculated using PHENIX, while the program Coot was utilized to add solvent molecules and make individual real space refinements of each residue when needed (48, 49). Figures were generated in the molecular graphical software, PyMol and protein-ligand interactions and bond lengths were determined using LigPlot Plus (50, 51). For the crystallographic data collection and refinement statics refer to Table 1. The apo- and Cu-CAII structures have been deposited to the PDB with accession numbers 6PEA and 6PDV, respectively.

The abbreviations used above include: Nitric oxide (NO), nitrite (NO₂⁻), carbonic anhydrase II (CAII), copper bound human CAII (Cu-CAII), Type 1 Cu site (T-1), Type 2 Cu site (T-2), endothelial nitrogen oxide synthase (eNOS), Amino Terminal Copper and Nickel (ATCUN).

TABLE 1
Data collection and refinement statistics.

| | Apo CAII | Cu CAII |
|---|---|---|
| Wavelength (Å) | 0.9795 | 0.9795 |
| Resolution range (Å) | 39.88-1.36 | 34.90-1.23 |
| | (1.41-1.36) | (1.27-1.23) |
| Space group | P 1 21 1 | P 1 21 1 |
| Unit cell: a, b, c (Å) | 41.3, 42.3, 72.0 | 41.2, 42.4, 72.0 |
| α, β, γ (°) | 90, 104.2, 90 | 90, 104.3, 90 |
| Total reflections | 163082 (11244) | 379527 (20966) |
| Unique reflections | 49501 (4044) | 68889 (6488) |
| Multiplicity | 3.3 (2.8) | 5.5 (3.2) |
| Completeness (%) | 95.9 (76.5) | 98.1 (92.8) |
| I/I_{σ} | 30.6 (6.3) | 12.9 (1.6) |
| Wilson B-factor (Å²) | 11.9 | 13.1 |
| R_{merge}^{a} (%) | 2.17 (17.24) | 8.98 (63.41) |
| R_{work}^{b} (%) | 14.86 (18.83) | 15.68 (27.71) |
| R_{free}^{c} (%) | 16.41 (20.91) | 17.41 (28.60) |
| Rₚᵢₘ^{d} (%) | 1.40 (12.18) | 3.92 (41.76) |
| Reflections used in refinement | 50381 (4045) | 68841 (6471) |
| Reflections used for R-free | 2005 (156) | 1846 (180) |
| Number of non-hydrogen atoms | 2375 | 2406 |
| macromolecules | 2156 | 2188 |
| ligands | 8 | 13 |
| solvent | 211 | 205 |
| Protein residues | 258 | 262 |
| RMS (bonds) (Å) | 0.007 | 0.014 |
| RMS (angles) (°) | 1.29 | 1.74 |
| Ramachandran favored (%) | 97.3 | 95.7 |
| Ramachandran allowed (%) | 2.7 | 4.3 |
| Ramachandran outliers (%) | 0 | 0 |
| Rotamer outliers (%) | 0.4 | 0 |
| Average B-factor (Å²) | 17.2 | 19.2 |
| macromolecules | 16.2 | 18.4 |
| ligands | 35.8 | 29.1 |
| solvent | 26.7 | 27.6 |

^{a}R_{merge} = (Σ|I − [symbol] |Σ [symbol] ) × 100.
^{b}R_{work} = (Σ|Fₒ − F_{c}|/Σ|Fₒ|) × 100.
^{c}R_{free} is calculated in the same way as R_{cryst} except it is for data omitted from refinement (5% of reflections for all data sets).
^{d}Rₚᵢₘ = [(Σ√1//N − 1)Σ|I − [symbol] |Σ [symbol] ] × 100.
Values in parentheses correspond to those of the highest-resolution shell.

### REFERENCES FOR EXAMPLE 1

1. Meldrum, N. U., and Roughton, F. J. W. (1933) Carbonic anhydrase. Its preparation and properties. J Physiol. 80, 113-142
2. Stadie, W. C., and O'Brien, H. (1933) The Catalysis of the Hydration of Carbon Dioxide and Dehydration of Carbonic Acid by an Enzyme Isolated from Red Blood Cells. J. Biol. Chem. 103, 521-529
3. Carbonic Anhydrase: Mechanism, Regulation, Links to Disease, | Susan C. Frost | Springer [online] http://www.springer.com/us/book/9789400773585 (Accessed Jul. 14, 2017)
4. Supuran, C. T. (2008) Carbonic anhydrases: novel therapeutic applications for inhibitors and activators. Nat Rev Drug Discov. 7, 168-181
5. Steiner, H., Jonsson, B. H., and Lindskog, S. (1975) The catalytic mechanism of carbonic anhydrase. Hydrogen-isotope effects on the kinetic parameters of the human C isoenzyme. Eur. J. Biochem. 59, 253-259
6. Moini, M., Demars, S. M., and Huang, H. (2002) Analysis of Carbonic Anhydrase in Human Red Blood Cells Using Capillary Electrophoresis/Electrospray Ionization-Mass Spectrometry. Analytical Chemistry. 74, 3772-3776
7. Briganti, F., Mangani, S., Scozzafava, A., Vernaglione, G., and Supuran, C. T. (1999) Carbonic anhydrase catalyzes cyanamide hydration to urea: is it mimicking the physiological reaction? J. Biol. Inorg. Chem. 4, 528-536
8. Nielsen, P. M., and Fago, A. (2015) Inhibitory effects of nitrite on the reactions of bovine carbonic anhydrase II with CO2 and bicarbonate consistent with zinc-bound nitrite. Journal of Inorganic Biochemistry. 149, 6-11
9. Mangani, S., and Hakansson, K. (1992) Crystallographic studies of the binding of protonated and unprotonated inhibitors to carbonic anhydrase using hydrogen sulphide and nitrate anions. European Journal of Biochemistry. 210, 867-871
10. Piazzetta, P., Marino, T., and Russo, N. (2017) Mechanistic Explanation of the Weak Carbonic Anhydrase's Esterase Activity. Molecules. 10.3390/molecules22061009
11. Andring, J. T., Lomelino, C. L., Tu, C., Silverman, D. N., McKenna, R., and Swenson, E. R. (2018) Carbonic anhydrase II does not exhibit Nitrite reductase or Nitrous Anhydrase Activity. Free Radic. Biol. Med. 117, 1-5
12. Aamand, R., Dalsgaard, T., Jensen, F. B., Simonsen, U., Roepstorff, A., and Fago, A. (2009) Generation of nitric oxide from nitrite by carbonic anhydrase: a possible link between metabolic activity and vasodilation. American Journal of Physiology-Heart and Circulatory Physiology. 297, H2068-H2074
13. Supuran, C. T. (2016) Structure and function of carbonic anhydrases. Biochemical Journal. 473, 2023-2032.
14. Fisher, Z., Kovalevsky, A. Y., Mustyakimov, M., Silverman, D. N., McKenna, R., and Langan, P. (2011) Neutron structure of human carbonic anhydrase II: a hydrogen-bonded water network “switch” is observed between pH 7.8 and 10.0. Biochemistry. 50, 9421-9423
15. Domsic, J. F., Avvaru, B. S., Kim, C. U., Gruner, S. M., Agbandje-McKenna, M., Silverman, D. N., and McKenna, R. (2008) Entrapment of Carbon Dioxide in the Active Site of Carbonic Anhydrase II. J Biol Chem. 283, 30766-30771
16. Silverman, D. N., and Lindskog, S. (1988) The catalytic mechanism of carbonic anhydrase: implications of a rate-limiting protolysis of water. Accounts of Chemical Research. 21, 30— 36
17. Maupin, C. M., McKenna, R., Silverman, D. N., and Voth, G. A. (2009) Elucidation of the proton transport mechanism in human carbonic anhydrase II. J. Am. Chem. Soc. 131, 7598— 7608
18. Feng, C. (2012) Mechanism of Nitric Oxide Synthase Regulation: Electron Transfer and Interdomain Interactions. Coord Chem Rev. 256, 393-411
19. Lundberg, J. O., Weitzberg, E., and Gladwin, M. T. (2008) The nitrate-nitrite-nitric oxide pathway in physiology and therapeutics. Nat Rev Drug Discov. 7, 156-167
20. Sparacino-Watkins, C. E., Lai, Y.-C., and Gladwin, M. T. (2012) Nitrate-nitrite-nitric oxide pathway in pulmonary arterial hypertension therapeutics. Circulation. 10.1161/CIRCULATIONAHA.112.107821
21. Li, Y., Hodak, M., and Bernholc, J. (2015) Enzymatic Mechanism of Copper-Containing Nitrite Reductase. Biochemistry. 54, 1233-1242
22. Strange, R. W., Dodd, F. E., Abraham, Z. H., Grossmann, J. G., Brüser, T., Eady, R. R., Smith, B. E., and Hasnain, S. S. (1995) The substrate-binding site in Cu nitrite reductase and its similarity to Zn carbonic anhydrase. Nat. Struct. Biol. 2, 287-292
23. Schultze, B., Lind, P. M., Larsson, A., and Lind, L. (2014) Whole blood and serum concentrations of metals in a Swedish population-based sample. Scandinavian Journal of Clinical and Laboratory Investigation. 74, 143-148
24. Hakansson, K., Wehnert, A., and Liljas, A. (1994) X-ray analysis of metal-substituted human carbonic anhydrase II derivatives. Acta Crystallogr. D Biol. Crystallogr. 50, 93-100
25. Hunt, J. A., Ahmed, M., and Fierke, C. A. (1999) Metal Binding Specificity in Carbonic Anhydrase Is Influenced by Conserved Hydrophobic Core Residues. Biochemistry. 38, 9054-9062
26. Antonyuk, S. V., Strange, R. W., Sawers, G., Eady, R. R., and Hasnain, S. S. (2005) Atomic resolution structures of resting-state, substrate- and product-complexed Cu-nitrite reductase provide insight into catalytic mechanism. Proc Natl Acad Sci USA. 102, 12041-12046
27. Fukuda, Y., Tse, K. M., Kado, Y., Mizohata, E., Matsumura, H., and Inoue, T. (2015) Insights into unknown foreign ligand in copper nitrite reductase. Biochem. Biophys. Res. Commun. 464, 622-628
28. Kim, C. U., Song, H., Avvaru, B. S., Gruner, S. M., Park, S., and McKenna, R. (2016) Tracking solvent and protein movement during CO2 release in carbonic anhydrase II crystals. Proc Natl Acad Sci USA. 113, 5257-5262
29. Kim, J. K., Lomelino, C. L., Avvaru, B. S., Mahon, B. P., McKenna, R., Park, S., and Kim, C. U. (2018) Active-site solvent replenishment observed during human carbonic anhydrase II catalysis. IUCrJ. 5, 93-102
30. Avvaru, B. S., Kim, C. U., Sippel, K. H., Gruner, S. M., Agbandje-McKenna, M., Silverman, D. N., and McKenna, R. (2010) A Short, Strong Hydrogen Bond in the Active Site of Human Carbonic Anhydrase II. Biochemistry. 49, 249-251
31. Saito, R., Sato, T., Ikai, A., and Tanaka, N. (2004) Structure of bovine carbonic anhydrase II at 1.95 Å resolution. Acta Crystallogr. D Biol. Crystallogr. 60, 792-795
32. Nettles, W. L., Song, H., Farquhar, E. R., Fitzkee, N. C., and Emerson, J. P. (2015) Characterization of the Copper(II) Binding Sites in Human Carbonic Anhydrase II. Inorg Chem. 54, 5671-5680
33. Lionetto, M. G., Caricato, R., Giordano, M. E., and Schettino, T. (2016) The Complex Relationship between Metals and Carbonic Anhydrase: New Insights and Perspectives. Int J Mol Sci. 10.3390/ijms17010127
34. An, H., Tu, C., Ren, K., Laipis, P. J., and Silverman, D. N. (2002) Proton transfer within the active-site cavity of carbonic anhydrase III. Biochim. Biophys. Acta. 1599, 21-27
35. Boone, C. D., Pinard, M., McKenna, R., and Silverman, D. (2014) Catalytic Mechanism of α-Class Carbonic Anhydrases: CO2 Hydration and Proton Transfer. in Carbonic Anhydrase: Mechanism, Regulation, Links to Disease, and Industrial Applications, pp. 31-52, Subcellular Biochemistry, Springer, Dordrecht, 10.1007/978-94-007-7359-2_3
36. Duda, D., Govindasamy, L., Agbandje-McKenna, M., Tu, C., Silverman, D. N., and McKenna, R. (2003) The refined atomic structure of carbonic anhydrase II at 1.05 A resolution: implications of chemical rescue of proton transfer. Acta Crystallogr. D Biol. Crystallogr. 59, 93-104
37. Marcus, R. A. (1964) Chemical and Electrochemical Electron-Transfer Theory. Annu. Rev. Phys. Chem. 15, 155-196
38. Silverman, D. N. (2000) Marcus rate theory applied to enzymatic proton transfer. Biochimica et Biophysica Acta (BBA)—Bioenergetics. 1458, 88-103
39. Application of Marcus theory for modeling proton transfer in cytochrome c oxidase—IOPscience [online] https://iopscience.iop.org/article/10.1088/1742-6596/917/4/042002 (Accessed Feb. 28, 2019)
40. Thompson, R. B., Maliwal, B. P., and Fierke, C. A. (1999) Selectivity and sensitivity of fluorescence lifetime-based metal ion biosensing using a carbonic anhydrase transducer. Anal. Biochem. 267, 185-195
41. McCall, K. A., and Fierke, C. A. (2000) Colorimetric and fluorimetric assays to quantitate micromolar concentrations of transition metals. Anal. Biochem. 284, 307-315
42. McCall, K. A., and Fierke, C. A. (2004) Probing Determinants of the Metal Ion Selectivity in Carbonic Anhydrase Using Mutagenesis. Biochemistry. 43, 3979-3986
43. Pinard, M. A., Boone, C. D., Rife, B. D., Supuran, C. T., and McKenna, R. (2013) Structural study of interaction between brinzolamide and dorzolamide inhibition of human carbonic anhydrases. Bioorg. Med. Chem. 21, 7210-7215
44. Tanhauser, S. M., Jewell, D. A., Tu, C. K., Silverman, D. N., and Laipis, P. J. (1992) A T7 expression vector optimized for site-directed mutagenesis using oligodeoxyribonucleotide cassettes. Gene. 117, 113-117
45. Uda, N. R., Seibert, V., Stenner-Liewen, F., Müller, P., Herzig, P., Gondi, G., Zeidler, R., Dijk, M. van, Zippelius, A., and Renner, C. (2015) Esterase activity of carbonic anhydrases serves as surrogate for selecting antibodies blocking hydratase activity. Journal of Enzyme Inhibition and Medicinal Chemistry. 30, 955-960
46. Kabsch, W. (2010) XDS. Acta Crystallogr. D Biol. Crystallogr. 66, 125-132
47. Evans, P. R., and Murshudov, G. N. (2013) How good are my data and what is the resolution? Acta Crystallogr D Biol Crystallogr. 69, 1204-1214
48. Adams, P. D., Afonine, P. V., Bunkóczi, G., Chen, V. B., Davis, I. W., Echols, N., Headd, J. J., Hung, L.-W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., and Zwart, P. H. (2010) PHENIX: a comprehensive Python-based system for macromolecular structure solution. Acta Crystallogr. D Biol. Crystallogr. 66, 213-221
49. Emsley, P., and Cowtan, K. (2004) Coot: model-building tools for molecular graphics. Acta Crystallogr. D Biol. Crystallogr. 60, 2126-2132
50. PyMOL Schrödinger, LLC
51. Laskowski, R. A., and Swindells, M. B. (2011) LigPlot+: multiple ligand-protein interaction diagrams for drug discovery. J Chem Inf Model. 51, 2778-2786
52. Duda, D., Tu, C., Qian, M., Laipis, P., Agbandje-McKenna, M., Silverman, D. N., and McKenna, R. (2001) Structural and Kinetic Analysis of the Chemical Rescue of the Proton Transfer Function of Carbonic Anhydrase II. Biochemistry. 40, 1741-1748

### Example 2. Metal Substituted Carbonic Anhydrase as a Nitrite Reductase

This example discusses nitrite reductase activity of CAII.

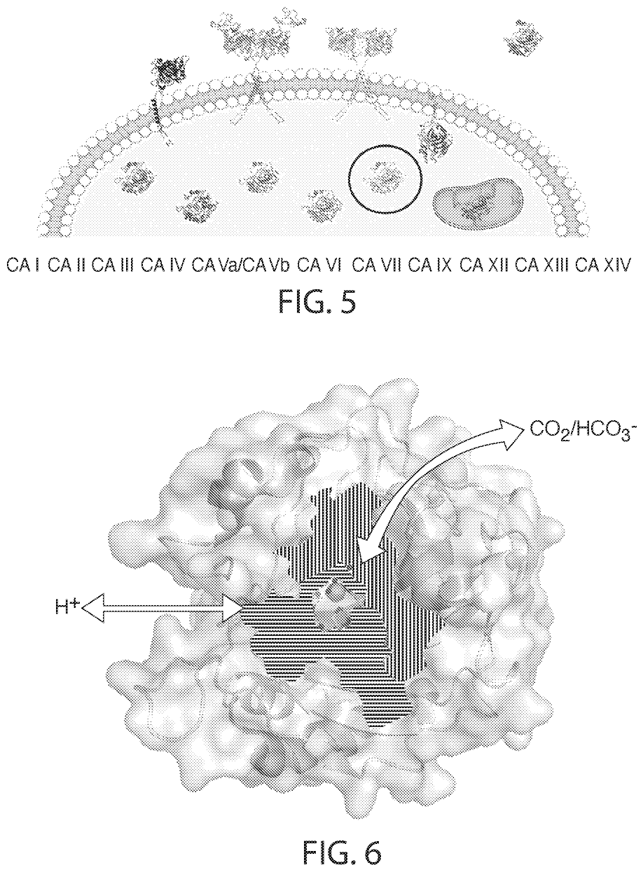

Humans produce 12 enzymatically active isoforms of carbonic anhydrase (CA, FIG. 5), with CAI and CAII being abundant in most cells. CAII is a metalloenzyme that catalyzes the reversible hydration of CO2 into HCO3⁻ (FIG. 6). CAII additionally catalyzes similar reactions of water with classes of other molecules such as esters, sulfates, and phosphates, demonstrating esterase, sulfatase and phosphatase activity, respectively, each of which have physiologically-relevant sequelae.

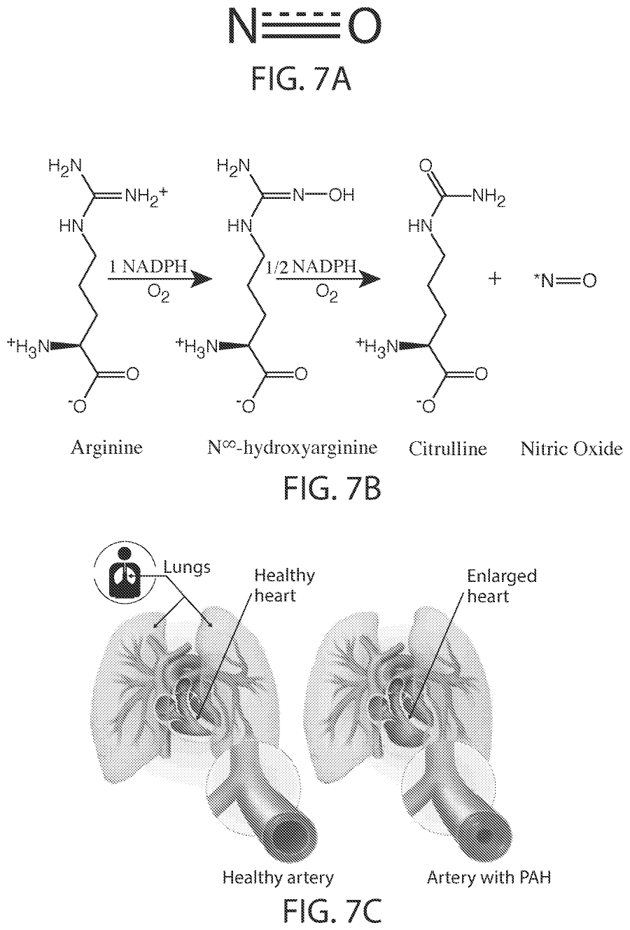

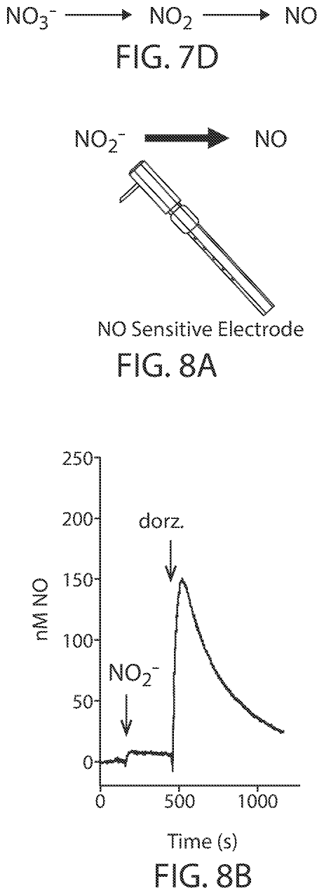

Given its abundance in blood, its catalytic activity and its potential role in nitric oxide (NO) formation, the possibility that CAII has nitrite reductase activity was investigated. Nitric oxide (FIG. 7A) stimulates smooth muscle relaxation resulting in vasodilation and as such has important clinical applications. Signaling pathways downstream of NO are targeted in a variety of heart diseases including pulmonary arterial hypertension (PAH), which is characterized by increased pulmonary vascular resistance and pulmonary artery pressures (FIG. 7C). Nitric oxide can be produced from arginine via nitric oxide synthetase catalysis (FIG. 7B), and from nitrite via the nitrite reductase pathway (FIG. 7D). Nitrite reductase activity is stimulated in humans under hypoxic conditions, though the enzyme or enzymes responsible, as well as the mechanisms of action, remain unknown. It was hypothesized that CAII may facilitate NO production via nitrite reductase activity in certain conditions.

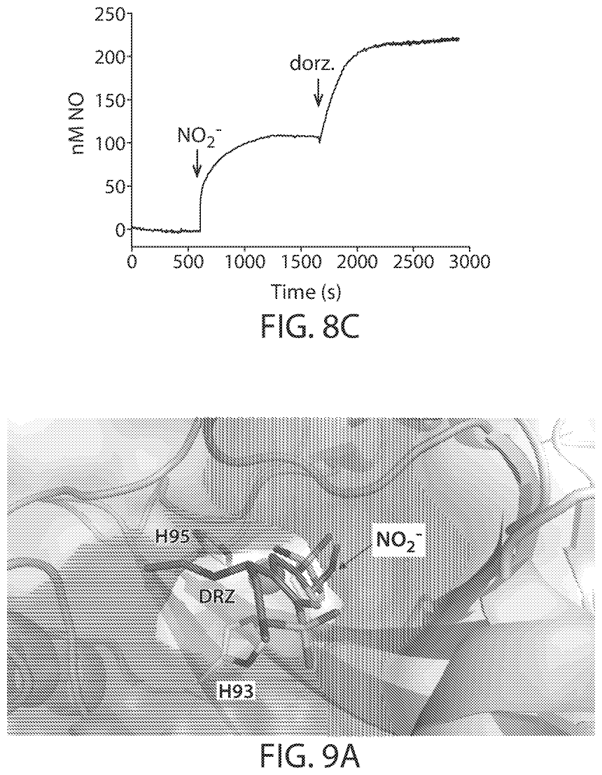

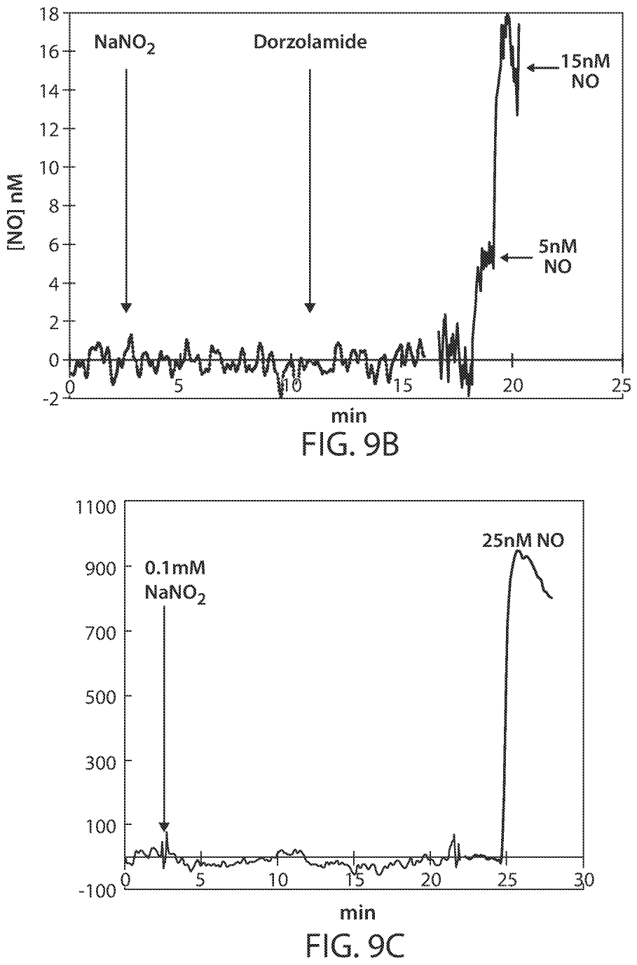

To facilitate testing nitrite reductase activity of CAII, NO-sensitive electrodes (FIG. 8A) were used to measure NO in CAII solutions containing NO₂⁻. Previous work demonstrated that nitrite reductase activity could be measured in this way, including in solutions containing NO₂⁻ spiked with dorzolamide, carbonic anhydrase inhibitor, at pH 7.2 (FIG. 8B) or pH 5.9 (FIG. 8C) as shown by Aamand, et al. (Am. J. Physiol. Heart Circ. Physiol. 297: H2068-H2074, 2009). Molecular modeling shows the potential interaction between Zn-CAII and nitrite in the presence of dorzolamide (FIG. 9A). Experiments demonstrated that in the absence of copper, CAII does not exhibit nitrite reductase or nitrous anhydrase activity, regardless of the presence or absence of dorzolamide. Measuring NO concentration with an electrode sensitive to NO (FIG. 9B) or via membrane inlet mass spectrometry (FIG. 9C) showed no production of NO by 100 uM CAII from 100 uM NO₂⁻.

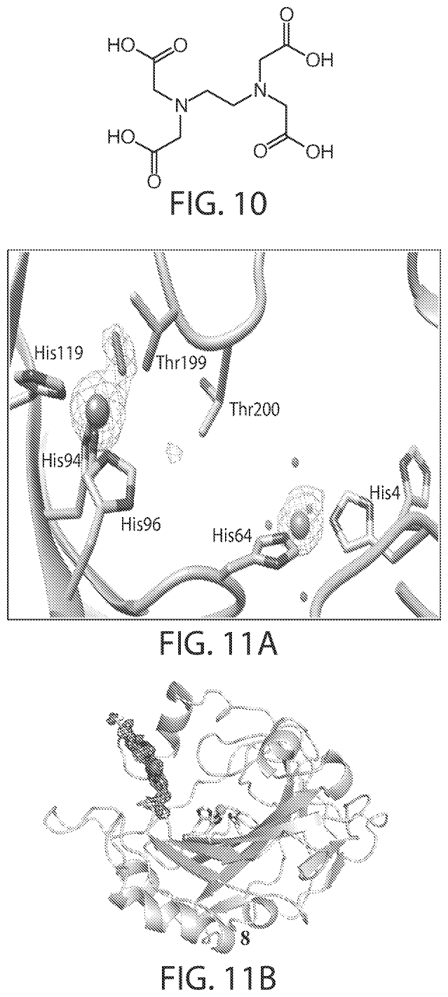

Previous work from Hanff et al. (Anal. Biochem. 550: 132-136, 2018) and Andring et al., (Free Radic. Biol. Med. 117: 1-5, 2018), both incorporated herein by reference, indicated that CAII in the presence of ethylenediaminetetraacetic acid (EDTA, FIG. 10) showed no nitrate reductase activity. These results demonstrated that specific metal cofactors are likely needed for nitrate reductase activity of CAII. Previous results from Ferraroni et al. indicated that CAII required both copper and another metal (e.g., zinc) (FIG. 11A) to have catalytic activity (J. Enzyme Inhib. Med. Chem., 33(1): 999-1005, 2018). As disclosed here, both metal coordination sites must be coordinated with copper (FIG. 11B) for CAII to have nitrite reductase activity.

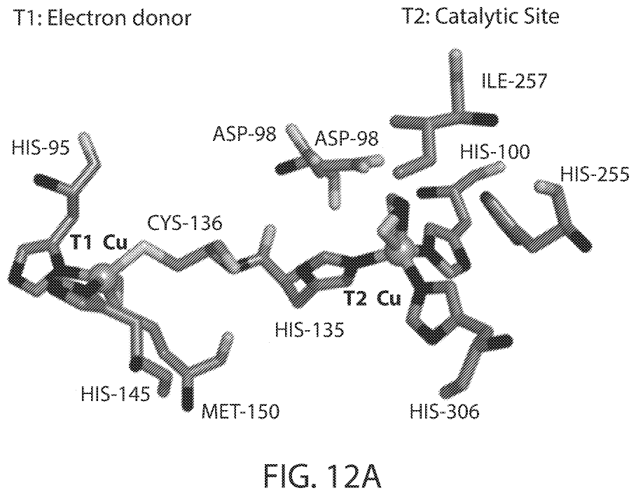

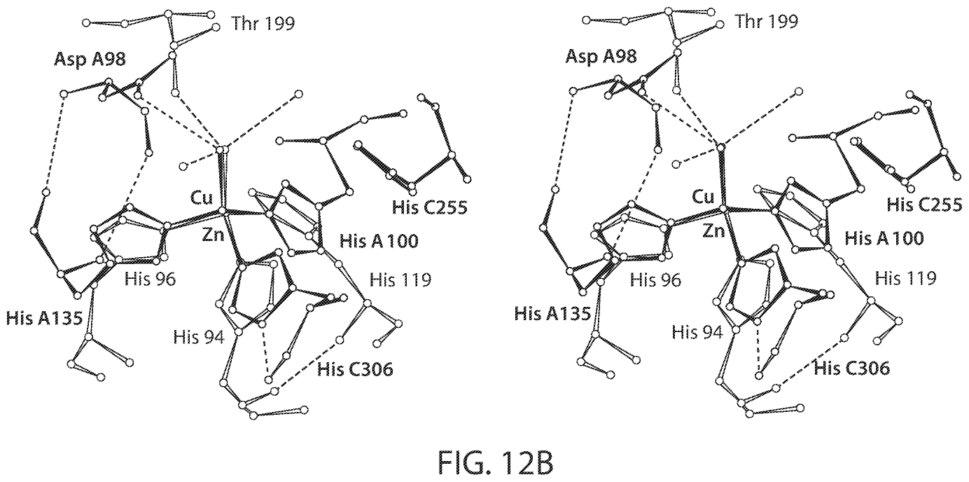

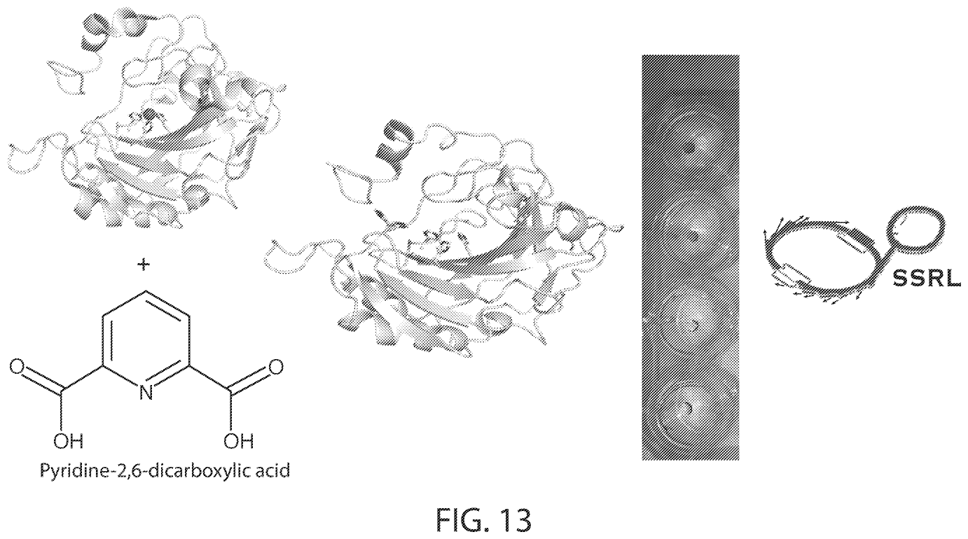

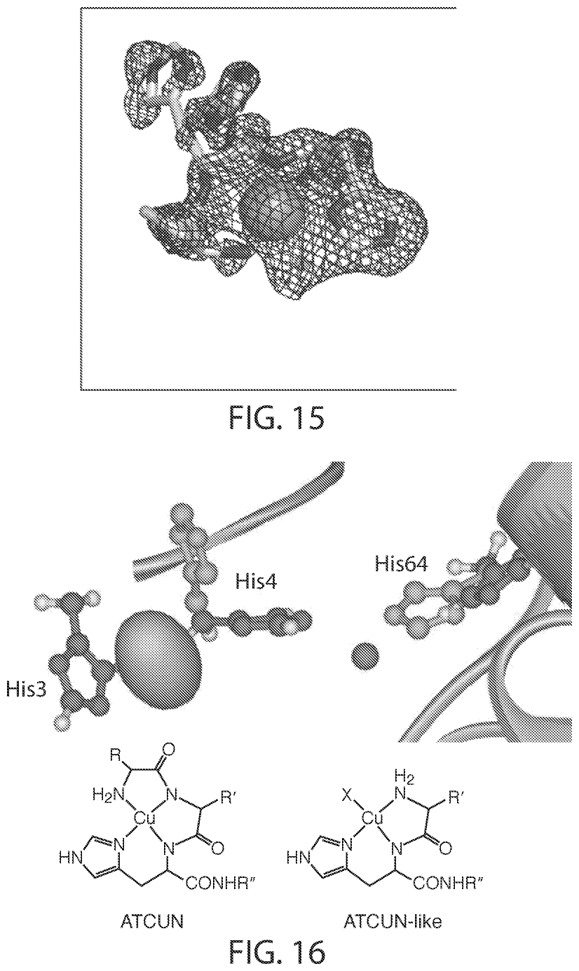

Nitrite reductases from various bacteria (e.g., FIG. 12A and FIG. 12B) coordinate copper in various sites. X-ray crystallography was used to determine copper coordination sites in CAII that might facilitate nitrite reductase activity. Pyridine-2,6-dicarboxylic acid was used to chelate metal ions from purified CAII, which was then mixed with copper to form crystals of Cu-CAII for crystallography (FIG. 13). Crystallography results of Apo-CAII (FIG. 14A), Zn-CAII (FIG. 14B), and Cu-CAII (FIG. 14C) demonstrate that, unlike with zinc which is only coordinated into one site of CAII, copper can be coordinated into two sites of CAII. Electron density plotting of the N-terminus of Cu-CAII (FIG. 15) demonstrates the finding disclosed here that CAII contains a second copper binding site which, unexpectedly, does not utilize His64. Nettles et al. (Inorg. Chem., 2015; 54(12):5671) previously predicted that the N terminus of CAII could gain order around a metal ion, but could not predict the coordination mode or the amino acid residues involved (FIG. 16).

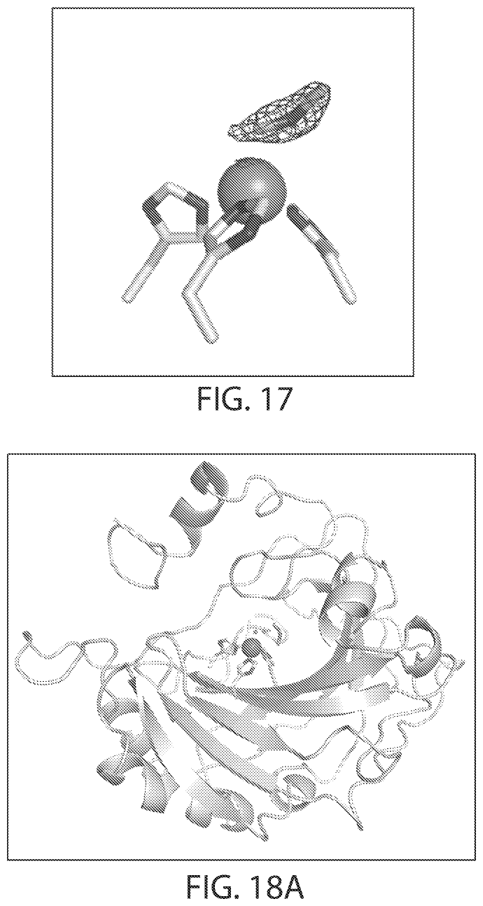

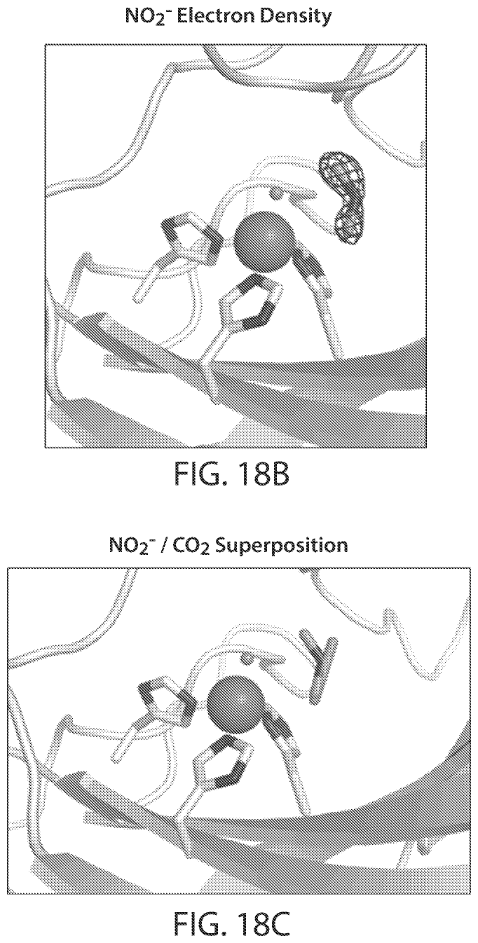

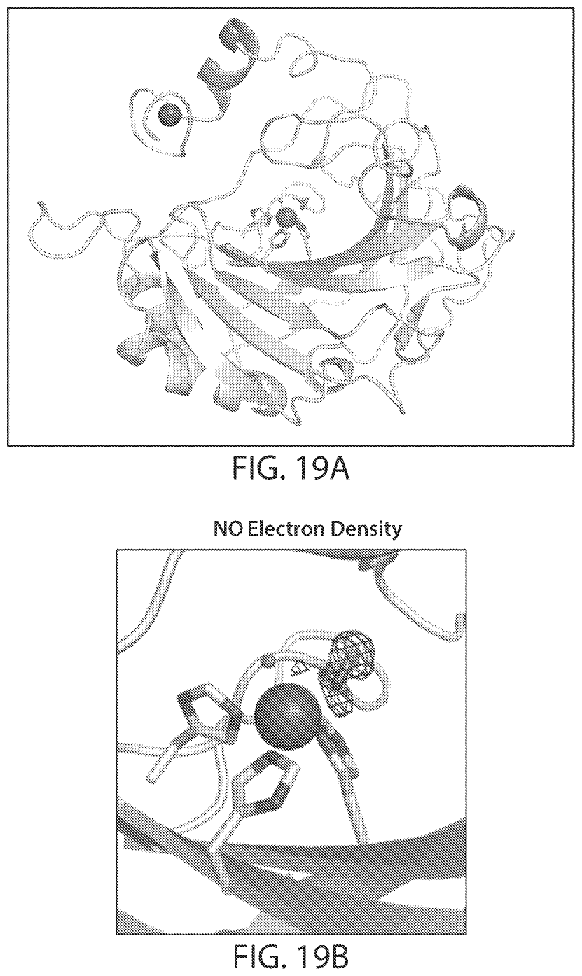

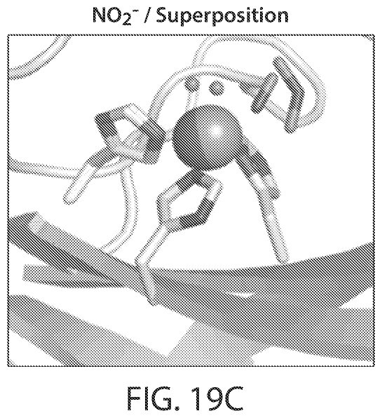

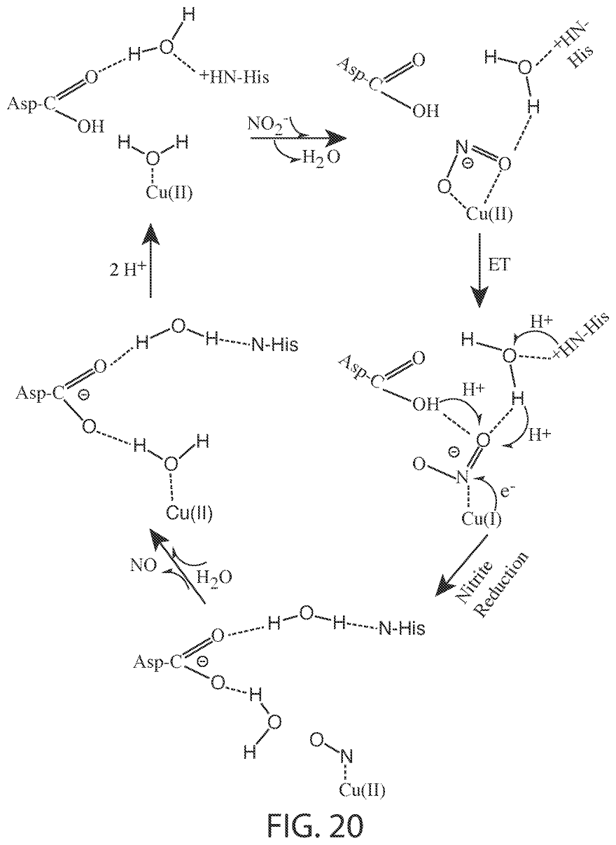

X-ray crystallography studies further demonstrated that endogenous NO₂⁻ binds to the T2 site of Cu-CAII (FIG. 17), whereas NO₂⁻ binds the T1 site in Zn-CAII (FIGS. 18A and 18B). Superposition of Zn-CAII bound to CO₂ and Zn-CAII bound to NO₂⁻ demonstrated that NO₂⁻ and CO₂ bind the same T1 site in Zn-CAII (FIG. 18C). Cu-CAII binds NO after soaking with NO₂⁻ (FIGS. 19A and 19B). Superposition of X-ray crystallography results from Zn-CAII and Cu-CAII incubated with NO₂⁻ demonstrate that the two metal ions interact differentially with CAII and further interact differentially with the ligand (FIG. 19C). Based on these findings, a mechanism was proposed for Cu-CAII catalyzed nitrite reduction (FIGS. 4A-4E), which can be compared to a previously proposed mechanism of nitrite reduction by copper-containing nitrite reductases (CuNiRs, FIG. 20, Li et al., Biochemistry 2015, 54(5): 1233-1242).

### Example 3. Aspirin: A Suicide Inhibitor of Carbonic Anhydrase II

This example discusses the esterase activity of CAII.

Carbonic Anhydrases (CAs) are a family of mainly zinc metalloenzymes responsible for the interconversion of carbon dioxide (CO₂) into bicarbonate (HCO₃⁻) and a proton via a ping-pong mechanism.¹ As such, CAs play an important role in blood homeostasis, CO₂/HCO₃⁻ transportation, and pH regulation.² There are 12 catalytic isoforms of CA expressed in humans, each with unique amino acid sequences, catalytic rates, cellular location, and tissue expression.² he active-site of human CAs is conserved, with a zinc ion coordinated by three histidine (H94, H96, and H119 (CAII numbering)) and a water/hydroxide.³ Of these isoforms, CAII is the most widely expressed isoform, responsible for regulating intracellular pH in nearly every cell.⁴ CAII is the fastest human CA, with a k_{cat} of ˜1100 ms⁻¹ that approaches the rate of diffusion.⁵

CAs play a critical role in physiology, to increase the rate of CO₂/HCO₃⁻ interconvertion.⁴ HCO₃⁻ is the most commonly transported form of CO₂ in the body.⁴ Large quantities of CO₂ are produced in tissues during respiration before removal by red blood cells (RBC) and transported to the lungs.⁴ While CAII plays a large role in transporting CO₂, it isn't the only mode of excretion. CAII expression levels are elevated in the kidney as it regulates HCO₃⁻ flux.⁶ CAII also balances cytoplasmic pH via interactions with a variety of membrane-bound ion carriers, including MCT1 and 4.⁴

In addition, CAII is important in blood homeostasis⁴. Human RBCs contain a high concentration of CAII at 0.8 attomol.⁷ CAII has also been shown to be involved in regulating platelet function. While the exact mechanism is unknown, CAII is known to be involved in nitrocysteine and nitric oxide formation, both critical in platelet inhibition.⁸

As CAs are responsible for a variety of physiological functions and pH regulation, they are often clinically targeted. CA inhibitors (CAIs) are used to treat a variety of diseases such as glaucoma, altitude sickness, and epilepsy.⁹ In addition, CAIs are currently being developed as anti-cancer drugs.¹⁰⁻¹² These inhibitors are designed to bind to the active site zinc, displacing the zinc bound solvent. The most common type of CAIs are sulfonamides, such as Acetazolamide, which has nM binding affinity. Many of these sulfonamide based molecules are used clinically such as Dorzolamide for the treatment of glaucoma.^{13,14} In addition to sulfonamides, a variety of other chemical motifs have been identified to inhibit CA, such as carboxylic acids.¹⁵ Nicotinic and Ferulic acid have recently been identified as inhibitors of CAII.¹⁶ Unlike the sulfonamide based drugs, these inhibitors do not directly displace the zinc bound solvent, but instead anchor through the solvent, blocking substrate entry to the active site.¹⁶ Furthermore, 3-nitro benzoic acid has also been reported as a potent CAI, with further studies showing its potential clinical relevance as a cancer therapeutic.¹⁷ These carboxylic acid based compounds represent a new and largely unstudied class of CAIs.

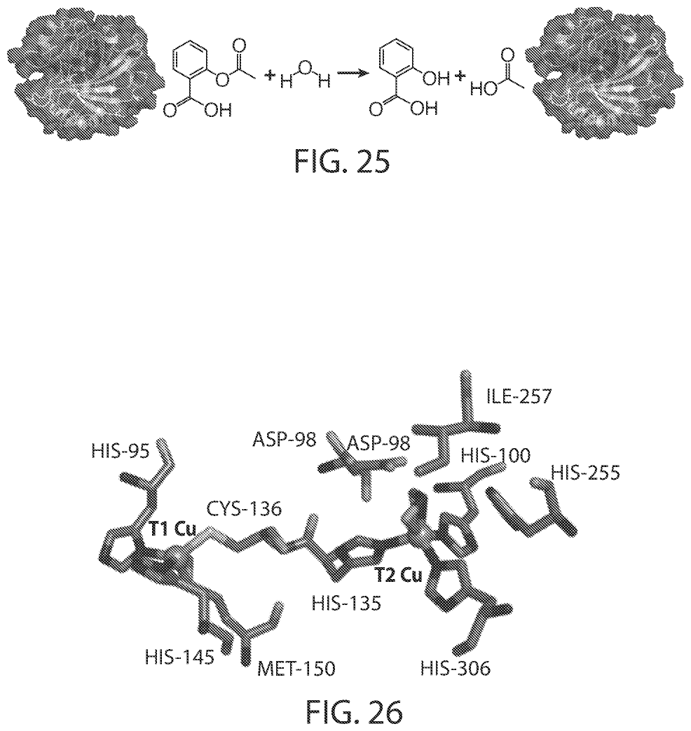

Aspirin (Acetylsalicylic acid) is one of the most widely studied and consumed drugs in use. Aspirin is a known COX (cyclooxygenase) inhibitor, giving the molecule its anti-inflammatory and blood thinning characterisitcs.¹⁸ Aspirin inhibits the COX enzymes by acetylating critical active site residues, leaving the enzymes acatalytic while generating salicylic acid (SA) as a byproduct.¹⁸ While Aspirin is typically used by patients prone to heart disease, there are many hypothesis about its other potential therapeutic benefits, such as a chemotherapy or a preventive of preeclampsia.¹⁸⁻²⁰ Each year, 40,000 metric tons of Aspirin are consumed which equates to ˜120 billion pills.²¹ A typical dose of Aspirin is 325 mg, however there are lower dosage options for everyday use and higher concentrations (up to 6 g per day, or ˜7 mM in blood) for at risk patients with heart disease.²¹ Interestingly, Aspirin only has a half-life of ˜15 minutes in blood due to a previously unidentified carboxylesterase.²² The short half-life of Aspirin leads to patients taking the drug daily to keep a therapeutic dose in their system. A recent study found in a genome wide search found that CAII is the only protein overexpressed in patients with Aspirin resistance and therefore may be the unidentified carboxylesterase.²³ Since Aspirin is a carboxylic acid based molecule, it was hypothesized that it could potentially bind to CAII. Here, this hypothesis was examined through structure activity relationship studies between Aspirin and CAII through X-ray crystallography and a spectroscopy based kinetic assay. It was determined that CAII is the previously unidentified carboxylesterase responsible for Aspirin's short half-life in the blood and that the product of this reaction (i.e., the hydrolysis of Aspirin facilitated by CAII), SA, can then inhibit CAII, thus making Aspirin a suicide inhibitor. FIG. 25 shows a schematic of the hydrolysis of CAII facilitated by Aspirin, the chemical reaction formula of which is shown below.

$$Aspirin + {H}_{2} O {⟹}^{CAII} SA \cdot CAII + Acetate$$

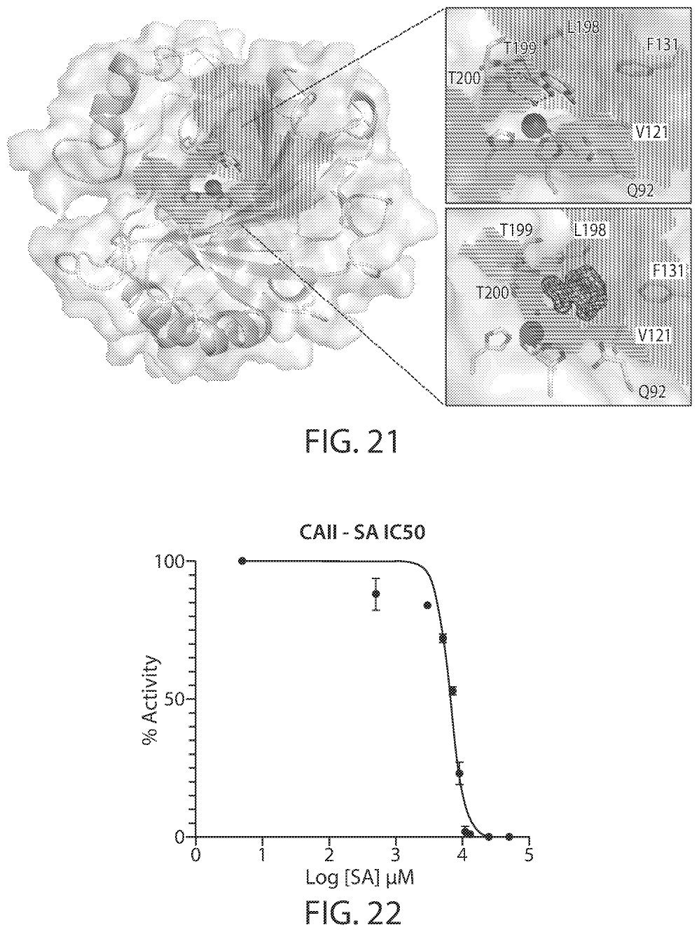

Based on previous studies with CAIs and the knowledge that CAII may be involved with Aspirin resistance, X-ray crystallography was utilized to determine if Aspirin can bind in the CAII active site. Suitable CAII crystals were grown using the sitting-drop, vapor diffusion method, and soaked with a solution of 50 mM Aspirin. The CAII crystals were well ordered and diffracted to 1.35 Å resolution. Upon data analysis and refinement, surprisingly SA was observed bound to the CAII active site instead of the expected Aspirin (FIG. 21). While the benzene ring and carboxylic acid group showed clear electron density, the ester linked acetate group was absent. Like the previously determined carboxylic acid-based inhibitor complexes, SA was shown to bind through the zinc bound solvent, and not the displacement of it. The carboxylic acid motif binds to the zinc bound solvent in the same orientation as the substrate CO_{2.}²⁴ SA is stabilized within the active site through interactions with residues on both the hydrophobic face and the hydrophilic face. On the hydrophilic face, the gatekeeper residue T199 as well as T200 form three hydrogen bonds with the carboxylic acid of SA. As well, Q92 forms distant dipole-dipole interactions with the hydroxyl of SA. On the hydrophobic face, residues V121, F131, and L198 form multiple Van der Waal interactions with the ring of SA. In addition, several other SA molecules were bound in various pockets on the surface of CAII, however these were involved in crystal lattice packing interactions and therefore not further discussed.

A known and widely studied function of CAs is their ability to act as an esterase.²⁵ When a small molecule with an ester bond such as Aspirin enters the active site of CAII, its ester bond is cleaved leaving an acetyl group and SA in the case of Aspirin. The zinc bound hydroxide is a strong nucleophile, able to attack the carbonyl of an ester, cleaving the bond. This esterase activity is often used to measure the activity of CAs, by monitoring the reaction via a colorimetric probe. 4-nitrophenyl acetate or pNPA is often used to monitor this reaction as its ester bond is cleavable by CAII and its product, 4-nitrophenol, is spectroscopically absorbent at 348 nm. Therefore, this molecule is used as a substrate of CAII and its cleavage is monitored by measuring absorbance at 348 nm. Inhibitors can then be added to determine their efficacy at inhibiting CAII.^{26,27}

Kinetic experiments were performed for both Aspirin and SA individually in the presence of CAII. Based on the crystallography experiments, it was predicted that Aspirin would act as a substrate firstly, then form SA thus acting as an inhibitor. The results from the preliminary kinetic assays with Aspirin however were inconclusive. The difference in absorption spectra between Aspirin and SA was problematic as the molecules absorbed differently at the experimental wavelength of 348 nm. Thus, the background absorbance of Aspirin and SA convoluted the absorbance from the esterase substrate, pNPA. Therefore, SA was tested by itself to circumvent the background absorbance from the Aspirin cleavage. At physiologically relevant concentrations of SA, it was shown that SA completely inhibited CAII (FIG. 22). Using Prism 8 software, the data was plotted to a non-linear regression to determine IC50.²⁸ The average standard deviation was 2.2% and the standard deviation specifically at 50% activity is 1.5%. With the small deviation in percent activity, the IC50 was 6.6+/−0.5 mM (FIG. 22). Therefore, at clinically prescribed high dosage of Aspirin, CAII can act as an Aspirin esterase to form SA, which can then act as a suicide inhibitor.

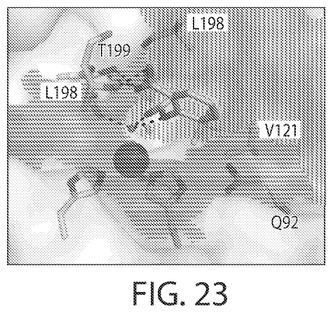

Using molecular modeling, it was estimated that Aspirin would bind in a similar fashion to Nicotinic and Ferulic acid within the active site of CAII, priming its ester group for nucleophilic attack (FIG. 23).¹⁶ The fast rate of conversion from Aspirin to SA has made it difficult to obtain the crystal structure of Aspirin-CAII complex.¹⁶

Similarly to SA, Aspirin was predicted to bind through the zinc bound solvent. The acetyl portion was bound to the zinc bound solvent based on the previously solved structure of acetate binding to CAII.²⁹ The carboxylic acid motif however, was positioned towards the hydrophilic pocket, interacting with T199 and T200. In the hydrophobic face, residues V121, V143, L198, and W209 form multiple Van der Waal interactions with the ring of Aspirin. The interaction with Q92 is conserved however F131 is positioned too far for interactions.

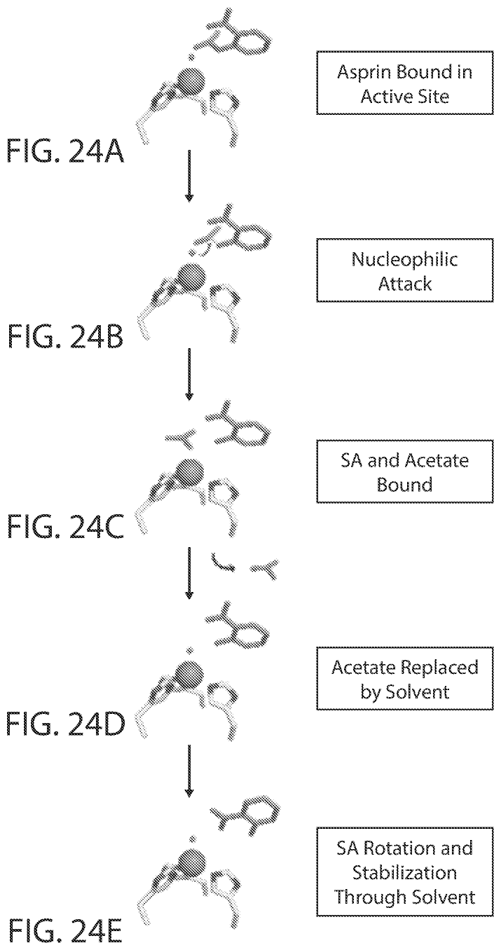

Based on the crystallographic data of SA bound to CAII and the modeling with Aspirin, a mechanism was proposed for CAII Aspirin ester cleavage and SA inhibition (FIGS. 24A-24E). Firstly, Aspirin binds to the zinc bound solvent within the active site with its acetate bound in a similar fashion of CO₂ binding, positioned for nucleophilic attack (FIG. 24A). The hydroxyl cleaves the ester bond in the Aspirin molecule leaving acetate bound to the active site (FIG. 24B). The acetate of the reaction is displaced by a water molecule that binds the zinc, while the SA remains in the active site (FIG. 24C-24D). Finally, the SA reorients within the active site and anchors through the zinc bound solvent, inhibiting any further reaction (FIG. 4E). This mechanism would explain how Aspirin initially acts as a substrate for CAII esterase activity, then its product, SA, is able to inhibit the enzyme.

Based on these findings, it was concluded that Aspirin binds and inhibits CAII via the SA product, as it retains the carboxylic acid motif similar to other CAIs such as Nicotinic, Ferulic, and 3-nitro benzoic acids.

### Methods

### Protein Expression and Purification

CAII was expressed and purified according to previously published protocols.³⁰⁻³² Competent BL21(DE3) cells were transformed with 1 μl plasmid DNA containing the CAII gene under expression control of T7 promoter. Cells were heat shocked for 45 seconds at 42° C., then placed on ice for two minutes. 350 μt of Luria broth (LB) was added and grown at 37° C. at 200 rpm for one hour. An overnight culture at 37° C. was used the following day for large scale growth until the OD600 reached ˜0.6. Protein expression was induced by the addition of 1 mL of 100 mg/mL Isopropyl-β-d-thiogalactopyranoside (IPTG) for three hours. 1 mL of 1M zinc sulfate was also added to aid in the folding of CAII. The culture was centrifuged for 10 minutes at 5000 rpm and the pellets were frozen overnight. The pellets were thawed and suspended with 40 mL of Wash Buffer 1 (WB1, 0.2M sodium sulfate, 0.1M Tris-HCl, pH 9.0). 40 mg of lysozyme and 5 mg of DNaseI were added into the bottle, then stirred at 4° C. for one hour. A microfluidizer lysed the cells before centrifugation at 12,000 rpm for one hour at 4° C. and then the supernatant was filtered with a 0.4 μm filter.

A p-aminomethylbenzenesulfonamide agarose resin affinity column was set up and equilibrated with WB1. The lysate was then loaded onto the column and washed with WB1 and Wash Buffer 2 (0.2M sodium sulfate, 0.1M Tris-HCl, pH 7.0) to elute non-specific proteins. The CA was eluted off the column with 0.4M sodium azide in 50 mM Tris-HCl, pH 7.8. Eluent was added to Amicon Ultra-15 centrifugal filter devices with a 10,000 kDa molecular weight cutoff and centrifuged at 6,000 rpm for 15 minutes, reducing the volume to ˜2 mL. 10 mL of storage buffer (50 mM Tris-HCl, pH 7.8, filtered) was added, spun at 6,000 rpm for 15 minutes, and the solution was resuspended. This was repeated five times to fully remove azide. Final protein concentration was checked by measuring the absorbance at 280 nm. A 12% SDS-PAGE gel was prepared to analyze protein purification purity.

### X-Ray Crystallography

Prior to crystallization, purified CAII was concentrated to 10 mg/ml via Amicon Ultra-15 centrifugal filters. CAII was crystallized via the hanging drop vapor diffusion method. 2.5 μL of 10 mg/mL protein was added to siliconized glass cover slips along with 2.5 μL of mother liquor consisting of 1.6M sodium citrate and 50 mM Tris at pH 7.8. 500 μL of mother liquor was added to the wells and grease was used to seal the glass clover slips to the wells.³³ Crystals formed within 24 hours. 500 mM Aspirin was purchased through Sigma Aldrich and Salicylic acid was purchased through Fisher Scientific. Each chemical was determined to be >99% purity through NMR and other assays. Aspirin was dissolved in 100% ethanol and a 1:10 dilution was made for a final concentration of 50 mM Aspirin in 10% ethanol. 1 μL of the Aspirin solution was added to the CAII drops and allowed to soak for 20 minutes.

The soaked Aspirin CAII crystals were harvested, flash frozen in liquid nitrogen, and shipped to Stanford Synchrotron Radiation Lightsource (SSRL). Data was collected at the 9-2 beamline at SSRL, using a Pilatus 6M detector with 0.15° oscillations, a wavelength of 0.9795 Å, and detector distance of 250 mm. Each data set consisted of 1200 images for a total of 180° data.

The diffraction images were indexed and integrated using XDS, then merged and scaled to the P21space group, using the program Aimless via the CCP4 program suite.³⁴⁻³⁶ The diffraction data was phased using the software package PHENIX utilizing the high resolution CAII PDB entry 3KS3 as the search model.³⁷ Coordinate refinements were calculated using PHENIX, while the program Coot was utilized to add solvent and SA.^{37,38} Coot was also utilized to make individual real space refinements of each residue where appropriate.³⁸ Aspirin modeling into hCAII was done in Chimera with MMTK providing minimization routines.³⁹ Performing an energy minimization for Aspirin allows for a more accurate depiction for how the drug may bind in the active site. Protein-inhibitor interactions were determined using LigPlot Plus and figures were made in the molecular graphical software PyMol.^{40,41}

### CA Inhibition Studies

Esterase assays were performed to measure the inhibition constants of Aspirin and SA on CAII using 4-nitrophenyl acetate as a colorimetric substrate. CAII cleaves the ester bond of 4-nitrophenyl acetate, generating 4-nitrophenol. The product, 4-nitrophenol, absorbs strongly at 348 nm, thus the reaction can be monitored spectroscopically.²⁵ CAII has high levels of esterase activity due to the nucleophilic nature of the zinc bound hydroxyl.

In a 96 deep-well plate, 50 μL of 0.1 mg/mL CAII (concentration in the 50 μL sample well) in storage buffer was added to each well. For inhibition studies, varying concentrations of inhibitors were preincubated with CAII at room temperature for 20 minutes prior to testing. To initiate the reaction, 200 μL of 0.8 mM pNPA dissolved in 3% acetone in water was added to the sample well. The well plate was then immediately inserted into Synergy HTX BioTek plate reader. Absorbance at 348 nm was recorded every 8 seconds for 10 minutes. 100 nM and 1000 nM acetazolamide were used as a positive control for inhibition. Inhibition with 100 nM acetazolamide showed 45% activity while 1000 nM showed 3.1% activity.

### REFERENCES FOR EXAMPLE 3

(1) Khalifah, R. G. The Carbon Dioxide Hydration Activity of Carbonic Anhydrase I. STOP-FLOW KINETIC STUDIES ON THE NATIVE HUMAN ISOENZYMES B AND C. J. Biol. Chem. 1971, 246 (8), 2561-2573.
(2) Lomelino, C. L.; Andring, J. T.; McKenna, R. Structural Insight into the Catalytic Mechanism of Carbonic Anhydrase. In Carbonic Anhydrases: Biochemistry, Mechanism of Action and Therapeutic Applications; Penttinen J., Ed.; Nova Science; 2018; p. 39.
(3) Eriksson, A. E.; Jones, T. A.; Liljas, A. Refined Structure of Human Carbonic Anhydrase II at 2.0 A Resolution. Proteins 1988, 4 (4), 274-282.
(4) Frost, S. C. Physiological Functions of the Alpha Class of Carbonic Anhydrases. In Carbonic Anhydrase: Mechanism, Regulation, Links to Disease, and Industrial Applications; Frost, S. C., McKenna, R., Eds.; Subcellular Biochemistry; Springer Netherlands: Dordrecht, 2014; pp 9-30.
(5) Steiner, H.; Jonsson, B.-H.; Lindskog, S. The Catalytic Mechanism of Carbonic Anhydrase. Eur. J. Biochem. 1975, 59 (1), 253-259.
(6) Brown, D.; Kumpulainen, T.; Roth, J.; Orci, L. Immunohistochemical Localization of Carbonic Anhydrase in Postnatal and Adult Rat Kidney. Am. J. Physiol. 1983, 245 (1), F110-118.
(7) Moini, M.; Demars, S. M.; Huang, H. Analysis of Carbonic Anhydrase in Human Red Blood Cells Using Capillary Electrophoresis/Electrospray Ionization-Mass Spectrometry. Anal. Chem. 2002, 74 (15), 3772-3776.
(8) Jakubowski, M.; Szahidewicz-Krupska, E.; Doroszko, A. The Human Carbonic Anhydrase II in Platelets: An Underestimated Field of Its Activity. BioMed Res. Int. 2018, 2018.
(9) Penttinen, J. Carbonic Anhydrases: Biochemistry, Mechanism of Action and Therapeutic Applications; Nova Science Publishers, Incorporated, 2018.
(10) Bozdag, M.; Carta, F.; Ceruso, M.; Ferraroni, M.; McDonald, P. C.; Dedhar, S.; Supuran, C. T. Discovery of 4-Hydroxy-3-(3-(Phenylureido)Benzenesulfonamides as SLC-0111 Analogues for the Treatment of Hypoxic Tumors Overexpressing Carbonic Anhydrase IX. J. Med. Chem. 2018, 61 (14), 6328-6338.
(11) Andreucci, E.; Ruzzolini, J.; Peppicelli, S.; Bianchini, F.; Laurenzana, A.; Carta, F.; Supuran, C. T.; Calorini, L. The Carbonic Anhydrase IX Inhibitor SLC-0111 Sensitises Cancer Cells to Conventional Chemotherapy. J. Enzyme Inhib. Med. Chem. 2019, 34 (1), 117-123.
(12) Safety Study of SLC-0111 in Subjects With Advanced Solid Tumours—Full Text View—ClinicalTrials.gov https://clinicaltrials.gov/ct2/show/NCT02215850
(13) Scozzafava, A.; Supuran, C. T. Glaucoma and the Applications of Carbonic Anhydrase Inhibitors. In Carbonic Anhydrase: Mechanism, Regulation, Links to Disease, and Industrial Applications; Subcellular Biochemistry; Springer, Dordrecht, 2014; pp 349-359.
(14) Nocentini, A.; Ceruso, M.; Bua, S.; Lomelino, C. L.; Andring, J. T.; McKenna, R.; Lanzi, C.; Sgambellone, S.; Pecori, R.; Matucci, R.; et al. Discovery of β-Adrenergic Receptors Blocker-Carbonic Anhydrase Inhibitor Hybrids for Multitargeted Antiglaucoma Therapy. J. Med. Chem. 2018, 61 (12), 5380-5394.
(15) Lomelino, C. L.; Supuran, C. T.; McKenna, R. Non-Classical Inhibition of Carbonic Anhydrase. Int. J. Mol. Sci. 2016, 17 (7).
(16) Lomelino, C. L.; McKenna, R. Carbonic Anhydrase II in Complex with Carboxylic Acid-Based Inhibitors. Acta Crystallogr. Sect. F Struct. Biol. Commun. 2019, 75 (3), 166-170.
(17) Cau, Y.; Vullo, D.; Mori, M.; Dreassi, E.; Supuran, C. T.; Botta, M. Potent and Selective Carboxylic Acid Inhibitors of Tumor-Associated Carbonic Anhydrases IX and XII. Mol. Basel Switz. 2017, 23 (1).
(18) Atallah, A.; Lecarpentier, E.; Goffinet, F.; Doret-Dion, M.; Gaucherand, P.; Tsatsaris, V. Aspirin for Prevention of Preeclampsia. Drugs 2017, 77 (17), 1819-1831.
(19) Drew, D. A.; Cao, Y.; Chan, A. T. Aspirin and Colorectal Cancer: The Promise of Precision Chemoprevention. Nat. Rev. Cancer 2016, 16 (3), 173-186.
(20) Ma, J.; Cal, Z.; Wei, H.; Liu, X.; Zhao, Q.; Zhang, T. The Anti-Tumor Effect of Aspirin: What We Know and What We Expect. Biomed. Pharmacother. Biomedecine Pharmacother. 2017, 95, 656-661.
(21) Kyle, M. E.; Wang, J. C.; Shin, J. J. Ubiquitous Aspirin: A Systematic Review of Its Impact on Sensorineural Hearing Loss. Otolaryngol.—Head Neck Surg. Off. J. Am. Acad. Otolaryngol.-Head Neck Surg. 2015, 152 (1), 23-41.
(22) Rowland, M.; Riegelman, S.; Harris, P. A.; Sholkoff, S. D. Absorption Kinetics of Aspirin in Man Follow Oral Administration of an Aqueous Solution. J. Pharm. Sci. 1972, 61 (3), 379-385.
(23) Jakubowski, M.; Dȩbski, J.; Szahidewicz-Krupska, E.; Turek-Jakubowska, A.; Gawryś, J.; Gawryś, K.; Skomro, R.; Derkacz, A.; Doroszko, A. Platelet Carbonic Anhydrase II, a Forgotten Enzyme, May Be Responsible for Aspirin Resistance. Oxid. Med. Cell. Longev. 2017.
(24) Domsic, J. F.; Avvaru, B. S.; Kim, C. U.; Gruner, S. M.; Agbandje-McKenna, M.; Silverman, D. N.; McKenna, R. Entrapment of Carbon Dioxide in the Active Site of Carbonic Anhydrase II. J. Biol. Chem. 2008, 283 (45), 30766-30771.
(25) Tashian, R. E.; Douglas, D. P.; Yu, Y.-S. L. Esterase and Hydrase Activity of Carbonic Anhydrase-I from Primate Erythrocytes. Biochem. Biophys. Res. Commun. 1964, 14 (3), 256-261.
(26) Uda, N. R.; Seibert, V.; Stenner-Liewen, F.; Müller, P.; Herzig, P.; Gondi, G.; Zeidler, R.; Dijk, M. van; Zippelius, A.; Renner, C. Esterase Activity of Carbonic Anhydrases Serves as Surrogate for Selecting Antibodies Blocking Hydratase Activity. J. Enzyme Inhib. Med. Chem. 2015, 30 (6), 955-960.
(27) Bua, S.; Lomelino, C.; Murray, A. B.; Osman, S. M.; ALOthman, Z. A.; Bozdag, M.; Abdel-Aziz, H. A.; Eldehna, W. M.; McKenna, R.; Nocentini, A.; et al. “A Sweet Combination”: Developing Saccharin and Acesulfame K Structures for Selectively Targeting the Tumor-Associated Carbonic Anhydrases IX and XII. J. Med. Chem. 2020, 63 (1), 321-333.
(28) Non-linear Regression, GraphPad Prism version 8.0.0 for Mac, GraphPad Software, San Diego, Calif. USA, www.graphpad.com
(29) Hakansson, K.; Briand, C.; Zaitsev, V.; Xue, Y.; Liljas, A. Wild-Type and E106Q Mutant Carbonic Anhydrase Complexed with Acetate. Acta CrystallogrSectD 1994, 50, 101-104.
(30) Avvaru, B. S.; Kim, C. U.; Sippel, K. H.; Gruner, S. M.; Agbandje-McKenna, M.; Silverman, D. N.; McKenna, R. A Short, Strong Hydrogen Bond in the Active Site of Human Carbonic Anhydrase II. Biochemistry 2010, 49 (2), 249-251.
(31) Tanhauser, S. M.; Jewell, D. A.; Tu, C. K.; Silverman, D. N.; Laipis, P. J. A T7 Expression Vector Optimized for Site-Directed Mutagenesis Using Oligodeoxyribonucleotide Cassettes. Gene 1992, 117 (1), 113-117.
(32) Pinard, M. A.; Boone, C. D.; Rife, B. D.; Supuran, C. T.; McKenna, R. Structural Study of Interaction between Brinzolamide and Dorzolamide Inhibition of Human Carbonic Anhydrases. Bioorg. Med. Chem. 2013, 21(22), 7210-7215.
(33) Diaz-Torres, N. A.; Mahon, B. P.; Boone, C. D.; Pinard, M. A.; Tu, C.; Ng, R.; Agbandje-McKenna, M.; Silverman, D.; Scott, K.; McKenna, R. Structural and Biophysical Characterization of the α-Carbonic Anhydrase from the Gammaproteobacterium Thiomicrospira crunogena XCL-2: Insights into Engineering Thermostable Enzymes for CO₂ Sequestration. Acta Crystallogr. D Biol. Crystallogr. 2015, 7/(Pt 8), 1745-1756.
(34) Kabsch, W. XDS. Acta Crystallogr. D Biol. Crystallogr. 2010, 66 (2), 125-132.
(35) Evans, P. R.; Murshudov, G. N. How Good Are My Data and What Is the Resolution? Acta Crystallogr. D Biol. Crystallogr. 2013, 69 (7), 1204-1214.
(36) Winn, M. D.; Ballard, C. C.; Cowtan, K. D.; Dodson, E. J.; Emsley, P.; Evans, P. R.; Keegan, R. M.; Krissinel, E. B.; Leslie, A. G. W.; McCoy, A.; et al. Overview of the CCP4 Suite and Current Developments. Acta Crystallogr. D Biol. Crystallogr. 2011, 67 (4), 235-242.
(37) Adams, P. D.; Afonine, P. V.; Bunkóczi, G.; Chen, V. B.; Davis, I. W.; Echols, N.; Headd, J. J.; Hung, L.-W.; Kapral, G. J.; Grosse-Kunstleve, R. W.; et al. PHENIX: A Comprehensive Python-Based System for Macromolecular Structure Solution. Acta Crystallogr. D Biol. Crystallogr. 2010, 66 (2), 213-221.
(38) Emsley, P.; Cowtan, K. Coot: Model-Building Tools for Molecular Graphics. Acta Crystallogr. D Biol. Crystallogr. 2004, 60 (12), 2126-2132.
(39) Pettersen, E. F.; Goddard, T. D.; Huang, C. C.; Couch, G. S.; Greenblatt, D. M.; Meng, E. C.; Ferrin, T. E. UCSF Chimera—A Visualization System for Exploratory Research and Analysis. J. Comput. Chem. 2004, 25 (13), 1605-1612.
(40) Laskowski, R. A.; Swindells, M. B. LigPlot+: Multiple Ligand—Protein Interaction Diagrams for Drug Discovery. J. Chem. Inf. Model. 2011, 51 (10), 2778-2786.
(41) PyMOL; Schrödinger, LLC.

### Example 4. Effects of Carbonic Anhydrase Inhibitors on Oxygen Consumption by Perfused Hearts

To test the effects of carbonic anhydrase inhibitors on oxygen consumption, Langendorff perfusion experiments were conducted using mouse hearts. Hearts were isolated from ˜12-week-old C57BL6 male mice and perfused ex vivo in Langendorff mode (retrograde perfusion via the aorta) with perfusate containing Krebs-Henseleit electrolytes with 2 mM acetate. The perfusate was constantly oxygenated with an oxygenator. An 8 mL bolus of solution containing 1 mM carbonic anhydrase inhibitor (acetazolamide or dorzolamide) was added to the Langendorff setup twice over the course of each measurement window. The inhibitor solution was oxygenated by bubbling with 95% O₂/5% CO₂ mixed gas prior to adding.

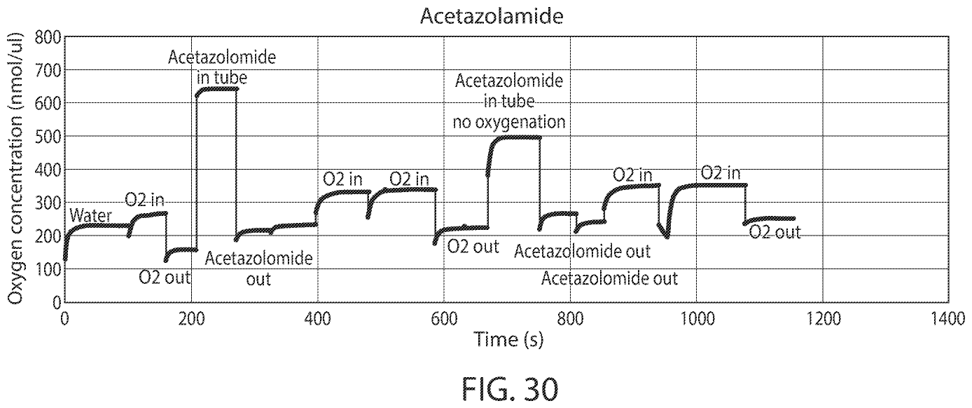

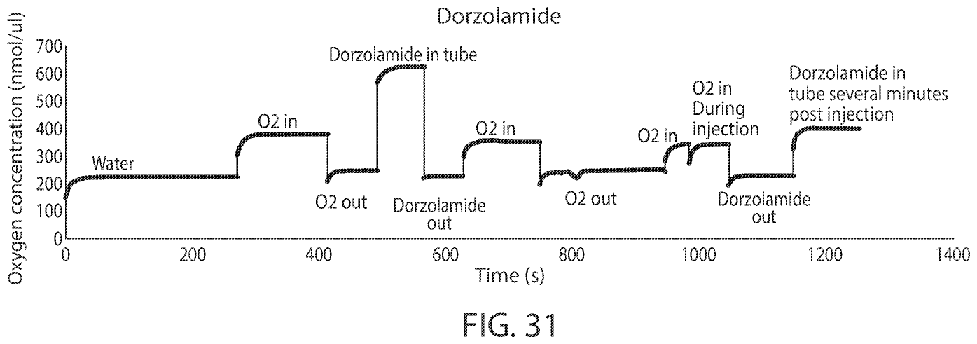

Dissolved oxygen concentration was measured in the perfusate, or in the carbonic anhydrase inhibitor solution, using an Oxygraph+ system. The raw oxygen concentration measurements are shown in FIG. 30 (acetazolamide) and FIG. 31 (dorzolamide). In each figure, “O2 in” refers to the oxygen concentration in the perfusate prior to entering the heart or in the carbonic anhydrase inhibitor solution, and “O2 out” refers to the oxygen concentration in the perfusate after it passes through the heart.

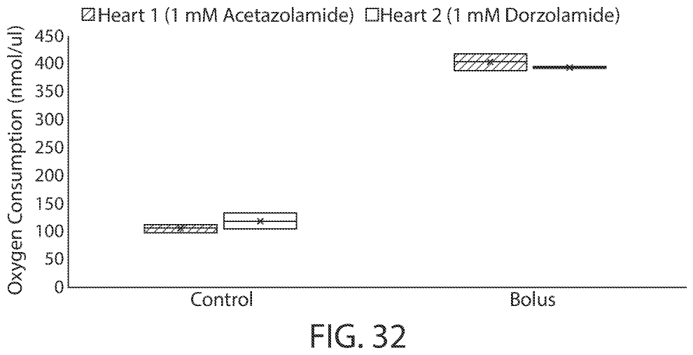

Oxygen consumption was measured as the difference between the O₂ in measurements and the O₂ out measurements, and results are shown in FIG. 32. The results demonstrate that treatment with acetazolamide or dorzolamide cause an increase in oxygen consumption by the heart. This suggests that carbonic anhydrase inhibitors can induce physiological effects in the heart by inducing vasodilation of cardiac blood vessels.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### SEQUENCES

### Example of Amino Acid Sequence of Carbonic Anhydrase II (CAII)

| (260 amino acids) | | |
|---|---|---|
| (SEQ ID NO: 1) | | |
| 1 | MSHHWGYGKH NGPEHWHKDF PIAKGERQSP VDIDTHTAKY DPSLKPLSVS YDQATSLRIL | 60 |
| 61 | NNGHAFNVEF DDSQDKAVLK GGPLDGTYRL IQFHFHWGSL DGQGSEHTVD KKKYAAELHL | 120 |
| 121 | VHWNTKYGDF GKAVQQPDGL AVLGIFLKVG SAKPGLQKVV DVLDSIKTKG KSADFTNFDP | 180 |
| 181 | RGLLPESLDY WTYPGSLTTP PLLECVTWIV LKEPISVSSE QVLKFRKLNF NGEGEPEELM | 240 |
| 241 | VDNWRPAQPL KNRQIKASFK | 260 |

### Example of Nucleic Acid Sequence Encoding Carbonic Anhydrase II (CAII)

(SEQ ID NO: 2)
Atgtcccatcactgggggtacggcaaacacaacggacctgagcactggc
ataaggacttccccattgccaagggagagcgccagtcccctgttgacat
cgacactcatacagccaagtatgacccttccctgaagcccctgtctgtt
tcctatgatcaagcaacttccctgaggatcctcaacaatggtcatgctt
tcaacgtggagtttgatgactctcaggacaaagcagtgctcaagggagg
acccctggatggcacttacagattgattcagtttcactttcactggggt
tcacttgatggacaaggttcagagcatactgtggataaaaagaaatatg
ctgcagaacttcacttggttcactggaacaccaaatatggggattttgg
gaaagctgtgcagcaacctgatggactggccgttctaggtatttttttg
aaggttggcagcgctaaaccgggccttcagaaagttgttgatgtgctgg
attccattaaaacaaagggcaagagtgctgacttcactaacttcgatcc
tcgtggcctccttcctgaatccttggattactggacctacccaggctca
ctgaccacccctcctcttctggaatgtgtgacctggattgtgctcaagg
aacccatcagcgtcagcagcgagcaggtgttgaaattccgtaaacttaa
cttcaatggggagggtgaacccgaagaactgatggtggacaactggcgc
ccagctcagccactgaagaacaggcaaatcaaagcttccttcaaataa

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B”, the application also contemplates alternative embodiments including “a composition consisting of A and B” and “a composition consisting essentially of A and B”.

What is claimed is:

1. A composition comprising carbonic anhydrase II (CAII) and copper, wherein the composition has nitrite reductase activity.

2. The composition of claim 1, wherein the copper is bound to the carbonic anhydrase.

3. The composition of claim 2, wherein His94, His96, and His119 of the CAII are bound to a copper atom, and His4, His3, and Ser2 of the CAII are bound to a copper atom.

4. The composition of any one of claims 1-3, further comprising a pharmaceutically acceptable carrier.

5. The composition of any one of claims 1-4 comprising a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a copper atom through His94, His96, and His119 of the CAII.

6. The composition of any one of claims 1-4 comprising a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a copper atom through His4, His3, and Ser2 of the CAII.

7. The composition of any one of claims 1-4 comprising a plurality of CAII molecules, wherein at least 10%, at least 20%, at least 30%, at least 40%, at least 50%, at least 60%, at least 70%, at least 80%, at least 90%, at least 95%, or at least 99% of the plurality of CAII molecules bind a first copper atom through His94, His96, and His119 of the CAII, and a second copper atom through His4, His3, and Ser2 of the CAII.

8. A method of making the composition of claim 1, comprising:
purifying CAII from a blood sample or culture of bacteria;
chelating metal ions from the purified CAII; and
incubating the purified CAII from which metal ions are chelated with copper at a molar ratio of 0.1:1 to 1:1 of CAII to copper.

9. The method of claim 8, wherein the chelating metal ions from the purified CAII comprises incubating the purified CAII with pyridine-2,6-dicarboxylic acid (DPA).

10. A composition comprising CAII, wherein the composition is prepared by:
purifying CAII from a blood sample or culture of bacteria;
chelating metal ions from the purified CAII; and
incubating with copper at a molar ratio of 0.1:1 to 1:1 of CAII to copper.

11. A method comprising administering to a subject the composition of any one of claims 1-4 or a composition prepared according to any one of the claims 8-10.

12. The method of claim 11, wherein the subject suffers from or is at risk of suffering from a condition that can be relieved by causing vasodilation.

13. The method of claim 12, wherein the condition that can be relieved by causing vasodilation is hypertension, pulmonary hypertension, a heart condition, erectile dysfunction, or muscular atrophy.

14. The method of claim 13, wherein the heart condition is heart failure, angina, coronary artery disease, or myocardial infarction.

15. The method of claim 14, where in the hypertension is primary hypertension or secondary hypertension, wherein the secondary hypertension is secondary to eclampsia, preeclampsia, renovascular disease or renal disease, sleep apnea, or endocrine abnormalities.

16. The method of any one of claims 11-15, wherein the composition is administered at a dose sufficient to increase the amount of copper-bound CAII in the subject by 10% or more.

17. A method comprising administering to a subject one or more inhibitors of carbonic anhydrase II (CAII), wherein the one or more inhibitors of CAII increases the nitrite reductase activity of Cu-bound CAII.

18. The method of claim 17, wherein the CAII is bound to Zn or to Cu.

19. The method of claim 17 or 18, wherein the one or more inhibitors preferentially inhibits CAII bound to Zn relative to CAII bound to Cu.

20. The method of any one of claims 17-19, wherein the one or more inhibitors of carbonic anhydrase II is/are sulfonamide-based carbonic anhydrase inhibitors.

21. The method of claim 20, wherein the sulfonamide-based carbonic anhydrase inhibitors is/are: acetazolamide, methazolamide, ethoxzolamide, dichlorphenamide, dorzolamide, brinzolamide, topiramate, celecoxib, sulpiride, sulthiame, valdecoxib, zonisamide, irosustat, an esterone sulfamate, or a benzyl-sulfonamide compound.

22. The method of any one of claims 17-20, wherein the subject suffers from or is at risk of suffering from a condition that can be relieved by causing vasodilation.

23. The method of claim 22, wherein the condition that can be relieved by causing vasodilation is hypertension, pulmonary hypertension, a heart condition, erectile dysfunction, or muscular atrophy.

24. The method of claim 23, wherein the heart condition is heart failure, angina, coronary artery disease or myocardial infarction.

25. The method of claim 23, where in the hypertension is primary hypertension or secondary hypertension, wherein the secondary hypertension is secondary to eclampsia, preeclampsia, renovascular disease or renal disease, sleep apnea, or endocrine abnormalities.

26. A method comprising administering to a subject who is administered or is going to be administered a nonsteroidal anti-inflammatory drug (NSAID) an inhibitor of carbonic anhydrase II (CAII), wherein the CAII has esterase activity.

27. The method of claim 26, wherein the NSAID is aspirin or ibuprofen.

28. The method of claim 26, wherein the NSAID is aspirin.

29. The method of any one of claims 26 to 28, wherein the inhibitor of carbonic anhydrase II is a sulfonamide-based carbonic anhydrase inhibitor.

30. The method of claim 29, wherein the one inhibitor of carbonic anhydrase II is: acetazolamide, methazolamide, ethoxzolamide, dichlorphenamide, dorzolamide, brinzolamide, topiramate, celecoxib, sulpiride, sulthiame, valdecoxib, zonisamide, irosustat, esterone sulfamate, or a benzyl-sulfonamide compound.

31. The method of any one of claims 26 to 30, wherein the subject has experienced a myocardial infarction, stroke, or Raynaud's phenomenon.

32. The method of any one of claims 26 to 31, wherein the subject is administered the CAII inhibitor simultaneously with being administered the NSAID, or within 4 hours of being administered the NSAID.
